(19) United States
(12) Patent Application Publication
(10) Pub. No.: US 2026/0091025 A1
(43) Pub. Date: Apr. 2, 2026

(54) BIOMOLECULES INVOLVED IN ALZHEIMER'S DISEASE

(71) Applicant: Electrophoretics Limited, London (GB)

(72) Inventors: Ian Hugo PIKE, London (GB); Malcolm Andrew WARD, London (GB); Claire Louise RUSSELL, London (GB); Vikram MITRA, London (GB)

(73) Assignee: Electrophoretics Limited, London (GB)

(21) Appl. No.: 19/284,132

(22) Filed: Jul. 29, 2025

Related U.S. Application Data
(63) Continuation of application No. 15/577,522, now Pat. No. 12,419,871.

(30) Foreign Application Priority Data
May 28, 2015 (GB) 1509134.1
Jul. 17, 2015 (GB) 1512596.6

(51) Int. Cl. A61K 31/437; A61K 31/13; A61K 31/445; A61K 31/55; A61K 31/57; A61P 25/28; C07K 14/47; C12N 9/16; G01N 33/68

(52) U.S. Cl. CPC A61K 31/437; A61K 31/13; A61K 31/445; A61K 31/55; A61K 31/57; A61P 25/28; C07K 14/4711; C12N 9/16; G01N 33/6896; G01N 2800/2821; G01N 2800/50; G01N 2800/56

(57) ABSTRACT

The invention relates to panels of biomarkers including proteins phosphatase 1 regulatory subunit 14A and/or 2′,3′-cyclic-nucleotide 3′-phosphodiesterase and/or phosphorylated tau or fragments thereof and methods using thereof for diagnosing, staging, treating and assessing the response of a treatment for a neurocognitive disorder characterised by tau toxicity, in particular for Alzheimer's disease. The present invention shows that the biomarkers disclosed herein are elevated in the brain of subjects with an advanced stage of a neurocognitive disorder (Braak stage V/VI) and/or are regulated in the CSF of AD subjects in comparison to cognitively affected non-AD controls; and/or regulated in response to two casein kinase 1 delta inhibitors.

### CROSS-REFERENCE TO RELATED APPLICATIONS

This application is a continuation of U.S. patent application Ser. No. 15/577,522, filed on Nov. 28, 2017, which is a U.S. National Stage Application of International Patent Application No. PCT/EP2016/062090 filed on May 27, 2016, which claims priority to GB Application No. 1509134.1, filed on May 28, 2015, and GB Application No. 1512596.6, filed on Jul. 17, 2015, the content of each of which is incorporated herein by reference in its entirety.

### REFERENCE TO AN ELECTRONIC SEQUENCE LISTING

The content of the electronic sequence listing (033945 00484 SL.xml; Size: 41,395 bytes; and Date of Creation: Jul. 10, 2025) is incorporated herein by reference in its entirety.

### FIELD OF THE INVENTION

The invention relates to sets of biomarkers and methods using thereof for diagnosing, staging, treating and assessing the response of a treatment for a neurocognitive disorder characterised by tau toxicity, in particular for Alzheimer's disease.

### BACKGROUND OF THE INVENTION

Neurofibrillary tangles, composed of intracellular aggregates of tau protein, are a key neuropathological feature of Alzheimer's disease (AD) and other neurodegenerative diseases, collectively termed tauopathies. Tau is an intracellular microtubule-associated protein, with several unique characteristics such as natively unfolded conformation, thermo-stability, acid-stability, and capability of post-translational modifications.

Abnormally hyperphosphorylated tau is a key feature of human tauopathies. In AD pathogenesis, accumulation of the amyloid-β peptide (Aβ) interacts with the signalling pathways that regulate the phosphorylation of tau. Hyperphosphorylation of tau disrupts its normal function in regulating axonal transport and leads to the accumulation of neurofibrillary tangles and toxic species of soluble tau.

Currently there is no cure for AD. Approved treatments are few and of limited efficacy, serving mostly to slow or delay progression.

Identification and development of new therapies for the treatment of AD and other tauopathies is also greatly affected by the lack of effective diagnostic, prognostic and predictive biomarkers and by the lack of new targets for the design of new therapies. Currently, AD can only be definitively diagnosed by brain biopsy or upon autopsy after a patient has died. Clearly, in clinical settings, brain biopsy is rarely performed and diagnosis is still primarily made based on the history of the symptoms and depends on a battery of neurological, psychometric and biochemical tests. These latter tests include assessment of ApoE e4 allele status and measurements of amyloid beta, tau and phospho-tau in cerebrospinal fluid.

These present methods, nevertheless, are still unsatisfactory not only for the early diagnosis of AD and other tauopathies, but also for predicting the progression of neurological disease which is important for recruitment of patients for clinical trials, for designing new therapies and for predicting the effectiveness of current and new therapies.

The ideal diagnostic biomarkers should have high specificity for disease versus non-disease and high sensitivity to distinguish between disease types and stages.

Prognostic biomarkers should reflect the intensity and severity of the pathological changes and predict their future course from a very early stage of the disease, before degeneration is observed, until advanced stages of the disease.

Pharmacodynamic biomarkers should give a reliable indication whether an administered therapy is efficacious based on the changes in the level of disease-related proteins in readily accessible body fluids such as blood, blood products including platelets, serum and most preferably plasma and CSF. It is also desirable that such pharmacodynamics biomarkers can provide guidance to clinicians when to stop treatment or switch to a different therapy.

New targets need to be efficacious, safe, meet clinical and commercial needs and, above all, be ‘druggable’. A ‘druggable’ target is accessible to the putative drug molecule, be that a small molecule or larger biologicals and upon binding, elicit a biological response which may be measured both in vitro and in vivo. In other words, its inhibition or activation will result in a therapeutic effect in a disease state.

Hence, there remains a need for proteins that may perform with superior sensitivity and/or specificity as biomarkers in the diagnosis, staging, prognostic monitoring and assessment of the effectiveness of treatments for patients with Alzheimer's disease and other tauopathies and may serve as new targets for the development of new therapies.

### SUMMARY OF THE INVENTION

The present invention, therefore, provides novel biomarkers and phosphorylated amino acids of tau or fragments thereof for use in methods for diagnosing, staging, treating and assessing the response of a treatment for a neurocognitive disorder characterised by tau toxicity such as tauopathies, in particular for Alzheimer's disease. In addition, the present invention provides novel targets for the development of new therapies against tauopathies or for repurposing existing therapies not originally designed for the treatment of neurocognitive disorders such as tauopathies.

In a first aspect the present invention provides for a panel of biomarkers comprising:
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform or a variant or a fragment thereof.

In a second aspect the present invention provides for a panel of biomarkers comprising tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422;
   wherein when the phosphorylated amino acid on tau is T181, the panel comprises tau or one or more fragments thereof having at least one more phosphorylated amino acid.

In a third aspect the present invention provides for a panel of biomarkers comprising one or more, optionally two or more proteins selected from Tables 5, 6, 7, 8, 9, 10, 11, 12, 13 or combinations thereof.

In one embodiment of the first aspect, the panel of biomarkers further comprises one or more, alternative two or more, proteins selected from Group A, which comprises Actin alpha cardiac muscle 1 comprising or having the amino acid sequence of SEQ ID NO:11, Antithrombin-III comprising or having the amino acid sequence of SEQ ID NO:12, BH3-interacting domain death agonist comprising or having the amino acid sequence of SEQ ID NO:3, cAMP-dependent protein kinase type I-beta regulatory subunit comprising or having the amino acid sequence of SEQ ID NO:24, Catenin delta-1 comprising or having the amino acid sequence of SEQ ID NO:4, Centrosomal protein of 170 kDa comprising or having the amino acid sequence of SEQ ID NO:23, Clathrin light chain B comprising or having the amino acid sequence of SEQ ID NO:5, Egl nine homolog 1 comprising or having the amino acid sequence of SEQ ID NO:13, Fibrinogen gamma chain comprising or having the amino acid sequence of SEQ ID NO:14, GMP reductase 1 comprising or having the amino acid sequence of SEQ ID NO:27, Guanine nucleotide-binding protein G(q) subunit alpha comprising or having the amino acid sequence of SEQ ID NO:6, Insulin-like growth factor-binding protein 6 comprising or having the amino acid sequence of SEQ ID NO:15, KxDL motif-containing protein 1 comprising or having the amino acid sequence of SEQ ID NO:28, Lambda-crystallin homolog comprising or having the amino acid sequence of SEQ ID NO:18, Myelin-associated oligodendrocyte basic protein comprising or having the amino acid sequence of SEQ ID NO:20, Neutral alpha-glucosidase AB comprising or having the amino acid sequence of SEQ ID NO:7, Nuclear pore complex protein Nup155 comprising or having the amino acid sequence of SEQ ID NO:19, OCIA domain-containing protein 1 comprising or having the amino acid sequence of SEQ ID NO:16, Protein KIAA1045 comprising or having the amino acid sequence of SEQ ID NO:25, Secernin-2 comprising or having the amino acid sequence of SEQ ID NO:8, Serum albumin comprising or having the amino acid sequence of SEQ ID NO:17, Short-chain specific acyl-CoA dehydrogenase comprising or having the amino acid sequence of SEQ ID NO:9, Synaptoporin comprising or having the amino acid sequence of SEQ ID NO:22, Syntaphilin comprising or having the amino acid sequence of SEQ ID NO:10, Transmembrane protein 119 comprising or having the amino acid sequence of SEQ ID NO: 21 and Tubulin alpha chain-like 3 comprising or having the amino acid sequence of SEQ ID NO:26.

In another embodiment, the panel of biomarkers according to the first aspect of the invention further comprises one or more proteins selected from Groups B, C or D.

In one other embodiment, the panel of biomarkers according to the first aspects and its embodiments may further comprise one or more biomarkers as defined in the second and/or third aspect of the invention.

In a fourth aspect the invention provides for a method for diagnosing a neurocognitive disorder in a subject, the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers of the panel in said sample with reference concentrations or amounts of said biomarkers.

In a fifth aspect the invention provides for method for staging a neurocognitive disorder in a subject, the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining the stage of the neurocognitive disorder in said subject by comparing said concentration or amount of each of the biomarkers of the panel in said sample with reference concentrations or amounts of said biomarkers.

In a sixth aspect the invention provides for a method for assessing in a subject the likelihood of developing a neurocognitive disorder, the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject is likely to develop a neurocognitive disorder by comparing said concentration or amount of each of the biomarkers of the biomarker panel in said sample with reference concentrations or amounts of said biomarkers.

In a seventh aspect the invention provides for a method for treating a neurocognitive disorder in a subject, the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers;
d) administering to said subject a drug for treating the neurocognitive disorder.

Alternatively, the seventh aspect of the present invention may be formulated as a drug for use in the treatment of a neurocognitive disorder in a subject, wherein the subject is selected by the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers.

Alternatively, the seventh aspect of the present invention may be formulated as a use of a drug for the manufacture of a medicament for the treatment of a neurocognitive disorder in a subject, wherein the subject is selected by the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers.

In an eighth aspect the present invention provides for a method for assessing the response to a drug for treating a neurocognitive disorder in a subject, wherein the subject has been treated or is being treated with said drug, the method comprising:
a) assaying a sample obtained from a subject for the biomarkers of the panel as defined in any one of the first, second and third aspect of the invention, including the embodiments thereof;
b) measuring in said sample a concentration or an amount of each of the biomarkers of said panel;
c) determining whether that subject has responded or is responding to said drug by comparing said concentration or amount of each of the biomarkers in said sample with reference concentrations or amounts of said biomarkers.

In one embodiment of the seventh and eighth aspects of the invention, the drug for treating a neurocognitive disorder is a kinase inhibitor. Preferably, the kinase inhibitor is selected from a tau kinase inhibitor or a casein kinase inhibitor, optionally a casein kinase 1 alpha, beta, gamma, delta or epsilon. More preferably the kinase inhibitor is a casein kinase 1 delta inhibitor selected from 5-(1,3-benzoxazol-2-yl)-4-(pyridin-4-yl)pyrimidin-2-amine; 2-amino-3-[(thiophen-2-yl)carbonyl]indolizine-1-carboxamide; 2-[3-(pyridin-4-yl)-1H-pyrazol-4-yl]-1,3-benzoxazole; 2-amino-3-[(4-fluorophenyl)carbonyl]indolizine-1-carboxamide; 2-Methyl-amino-3-[(4-fluorophenyl)carbonyl]indolizine-1-carboxamide; 2-amino-3-benzoylindolizine-1-carboxamide; 2-amino-1-[(4-fluorophenyl)carbonyl]-1H-indole-3-carboxamide; combinations thereof; or pharmaceutically acceptable salt or solvate thereof. Even more preferably, the casein kinase 1 delta inhibitor is selected from 5-(1,3-benzoxazol-2-yl)-4-(pyridin-4-yl)pyrimidin-2-amine; 2-amino-3-[(4-fluorophenyl)carbonyl]indolizine-1-carboxamide; 2-Methyl-amino-3-[(4-fluorophenyl)carbonyl]indolizine-1-carboxamide.

In preferred embodiments of the fourth, fifth, sixth, seventh and eighth aspect of the invention, the neurocognitive disorder is characterised by tau toxicity. More preferably, the neurocognitive disorder is a tauopathy. The tauopathy may be selected from the group of Alzheimer's disease, frontotemporal dementia with Parkinsonism linked to chromosome 17 (FTDP-17), progressive supranuclear palsy (PSP), Pick's disease, corticobasal degeneration, multisystem atrophy (MSA), neurobasal degeneration with iron accumulation, type 1 (Hallervorden-Spatz), argyrophilic grain dementia, Down's syndrome, diffuse neurofibrillary tangles with calcification, dementia pugilistica, Gerstmann-Straussler-Scheinker disease, myotonic dystrophy, Niemann-Pick disease type C, progressive subcortical gliosis, prion protein cerebral amyloid angiopathy, tangle only dementia, postencephalitic parkinsonism, subacute sclerosing panencephalitis, Creutzfeldt-Jakob disease, amyotrophic lateral sclerosis/parkinsonism-dementia complex, non-Guamanian motor neuron disease with neurofibrillary tangles/dementia, chronic traumatic encephalopathy, alpha-synucleinopathies, Parkinson's disease or combinations thereof.

Even more preferably the tauopathy is Alzheimer's disease.

In one embodiment of the seventh aspect of the invention, step d) further comprises administering an additional therapeutic agent selected from the group of memantine (e.g. Namenda®), galantamine (e.g. Razadyne®), rivastigmine (e.g. Exelon®), donepezil (e.g. Aricept®), solanezumab, 5HT₅ antagonists or combinations thereof.

In one other embodiment of the eighth aspect of the invention the subject may have also been treated or may also being treated with an additional therapeutic agent selected from the group of memantine (e.g. Namenda®), galantamine (e.g. Razadyne®), rivastigmine (e.g. Exelon®), donepezil (e.g. Aricept®), solanezumab, 5HT₅ antagonists or combinations thereof.

In another embodiment of the eighth aspect, preferably or alternatively, after step c), the method comprises administering an additional therapeutic agent selected from the group of memantine (e.g. Namenda®), galantamine (e.g. Razadyne®), rivastigmine (e.g. Exelon®), donepezil (e.g. Aricept®), solanezumab, 5HT₅ antagonists or combinations thereof.

In embodiments of the fourth, fifth, sixth, seventh and eighth aspect of the invention the assaying step a) and/or the measuring step b) comprise:
i) contacting said sample with one or more binding agents to each of said biomarkers of the panel; or
ii) detecting in said sample autoantibodies specific to each of said biomarkers; or
iii) detecting in said sample by mass spectrometry each of said biomarkers of the panel or fragments thereof, optionally by previously labelling said sample with one or more isobaric reactive mass labels; or
iv) detecting in said sample by 2D gel electrophoresis each of said biomarkers of the panel; or
iv) any combinations of i), ii), iii) or iv).

Preferably, the assaying in step a) and/or the measuring is step b) comprise:
i) detecting one or more fragments of said biomarkers in the panel and/or
ii) detecting one or more phosphorylated amino acids on tau comprising or having the amino acid sequence of SEQ ID NO: 29 or one or more fragments thereof; wherein when the phosphorylated amino acid on tau to be detected is T181, at least one more phosphorylated amino acid on tau or one or more fragments thereof is detected.

More preferably, the sample is immobilised on a solid support.

In embodiments of the fourth, fifth, sixth, seventh and eighth aspect of the invention the sample is selected from the group of cerebrospinal fluid (CSF), blood, plasma, serum, saliva, urine, tissue (e.g. brain tissue) or combinations thereof. Preferably the sample is CSF or blood. Also preferably, the subject is a human subject.

In a ninth aspect the present invention provides for a kit comprising reagents for assaying and/or measuring in a sample the biomarkers of a panel as defined in any one of the first, second and third aspect of the invention, including the embodiments thereof.

The reagents may comprise one or more binding agents which specifically bind to the biomarkers of the panels. Preferably, the one or more binding agents are primary antibodies, wherein each primary antibody specifically binds to:
i) a different biomarkers of the panel and/or
ii) one or more phosphorylated amino acids of tau comprising or having amino acid sequence of SEQ ID NO: 29 or fragments thereof.

The reagents may further comprise one or more secondary antibodies which specifically bind to said primary antibodies. Preferably the secondary antibodies are labelled.

In another embodiment of the ninth aspect and of its embodiments the sample is selected from the group of cerebrospinal fluid (CSF), blood, plasma, serum, saliva, urine, tissue (e.g. brain tissue) or combinations thereof.

### BRIEF DESCRIPTION OF THE DRAWINGS

FIG. 1. Sequence of human protein phosphatase 1 regulatory subunit 14A. Amino acids flagged by symbol □ or a * indicate amino acids which are replaced by a different or a modified amino acid in an isoform or a variant of human protein phosphatase 1 regulatory subunit 14A, respectively.

FIG. 2. Sequence of human 2′,3′-cyclic-nucleotide 3′-phosphodiesterase. Amino acids flagged by symbol □ or a * indicate amino acids which are replaced by a different or a modified amino acid in an isoform or a variant of human 2′,3′-cyclic-nucleotide 3′-phosphodiesterase, respectively.

FIG. 3. Venn diagram for protein phosphatase 1 regulatory subunit 14A.

FIG. 4. Venn diagram for 2′,3′-cyclic-nucleotide 3′-phosphodiesterase.

FIG. 5. Levels (as sums of ions intensities of all non-modified peptides) of protein phosphatase 1 regulatory subunit 14A in (A) Human brain with mild (Braak 0-II) (n=3), moderate (Braak III/IV) or severe (Braak v/VI) tau pathology; (B) Mouse brain from the TMHT model of human tauopathy treated orally with vehicle alone or vehicle including 30 mg/kg of casein kinase 1 delta inhibitors 5-(1,3-benzoxazol-2-yl)-4-(pyridin-4-yl)pyrimidin-2-amine (PS110) and 2-Methyl-amino-3-[(4-fluorophenyl)carbonyl]indolizine-1-carboxamide (PS278-05); and (C) CSF from cognitively affected non-AD controls (CTL; n=3) and patients with biochemically diagnosed AD (AD; n=3).

FIG. 6. Levels (as sums of ions intensities of all non-modified peptides) of 2′,3′-cyclic-nucleotide 3′-phosphodiesterase in (A) Human brain with mild (Braak 0-II) (n=3), moderate (Braak III/IV) (n=3) or severe (Braak v/VI) (n=3) tau pathology; (B) Mouse brain from the TMHT model of human tauopathy treated orally with vehicle alone (n=3) or vehicle including 30 mg/kg of casein kinase 1 delta inhibitors 5-(1,3-benzoxazol-2-yl)-4-(pyridin-4-yl)pyrimidin-2-amine (PS110; n=3) and 2-Methyl-amino-3-[(4-fluorophenyl)carbonyl]indolizine-1-carboxamide (PS278-05; n=3); and (C) cerebrospinal fluid from cognitively affected non-AD controls (CTL; n=3) and patients with biochemically diagnosed AD (AD; n=3).

FIG. 7. Experimental design of TMTcalibrator™ experiments to measure brain derived proteins in CSF of human control and biochemically diagnosed AD subjects.

### DEFINITIONS

The term “biomarker(s)” includes all biologically relevant forms of the protein identified, including post-translational modifications. For example, the biomarker can be present in a glycosylated, phosphorylated, multimeric, fragmented or precursor form. A biomarker fragment may be naturally occurring or, for example, enzymatically generated and still retaining the biologically active function of the full protein. Fragments will typically be at least about 10 amino acids, usually at least about 50 amino acids in length, and can be as long as 300 amino acids in length or longer.

The term “canonical sequence” is used herein as to refer to the most prevalent sequence and/or the most similar sequence among orthologous species. In particular, unless otherwise specified, the canonical sequence refers herein to the human sequence.

The term “KEGG pathway” refers to a collection of manually drawn pathway maps representing molecular interactions and reaction networks for metabolism, genetic information processing, environmental information processing, cellular processes, organismal systems, human diseases and drug development. “KEGG pathways mapping” is the process to map molecular datasets, especially large-scale datasets in genomics, transcriptomics, proteomics, and metabolomics, to the KEGG pathway maps for biological interpretation of higher-level systemic functions; (http://www.genome.jp/kegg/pathway.html).

The term “concentration or amount” refers to the relative concentration or amount of biomarker in the sample, for example as determined by LC-MS/MS label free quantification approaches such as area under the curve and spectral counting.

The term “comparing” or “compare” or grammatical equivalents thereof, means measuring the relative concentration or amount of a biomarker in a sample relative to other samples (for example protein concentrations or amounts stored in proprietary or public database).

The term “reference concentration or amount” refers to, but it is not limited to, protein concentrations or amounts stored in proprietary or public databases. The “reference concentration or amount” may have been obtained from a large screening of patients, or by reference to a known or previously determined correlation between such a determination and clinical information in control patients. For example, the reference values may be determined by comparison to the concentration or amount of the biomarkers in a control subject, for example a healthy person (i.e. without dementia) of similar age and gender as the subject. Alternatively, the reference values are values which can be found in literature such as the ApoE ε4 allele presence whereby the presence or absence of the mutations at position 112 and 158 represent the reference to be compared to, or like the levels of total tau (T-tau)>350 ng/L, phospho-tau (P-tau)>80 ng/L and Aβ42<530 ng/L in the CSF (Hansson 0, et al., Lancet Neurol. 2006, 5(3), 228-34). In addition, the reference values may have been obtained from the same subject at one or more time points which precede in time the test time point. Such earlier sample may be taken one week or more, one month or more, three months or more, most preferably six months or more before the date of the test time point. In some embodiments, multiple earlier samples may be compared in a longitudinal manner and the slope of change in biomarker expression may be calculated as a correlate of cognitive decline.

The term “control” or as used herein “non AD control” or “non AD subject” refers to a tissue sample or a bodily fluid sample taken from a human or non-human subject that is cognitively normal or diagnosed with or presenting symptoms of a cognitive abnormality but defined, with respect to the existing biochemical tests, as non AD subjects.

The term “binding agent” generally refers to any molecule that has affinity for a biomarker of the present invention. Binding agents may comprises apatamers, antibodies, lectins and enzymes.

The term “antibody” includes polyclonal antiserum, monoclonal antibodies, fragments of antibodies such as single chain and Fab fragments, and genetically engineered antibodies. The antibodies may be chimeric or of a single species.

The term “aptamer” includes small affinity agents with selectivity for a specific target and which are polymers of nucleic acids, amino acids or other synthetic organic molecules or combinations of any of these constituent molecules.

The terms “selected reaction monitoring”, “SRM” and “MRM” means a mass spectrometry assay whereby precursor ions of known mass-to-charge ratio representing known biomarkers are preferentially targeted for analysis by tandem mass spectrometry in an ion trap or triple quadrupole mass spectrometer. During the analysis the parent ion is fragmented and the number of daughter ions of a second predefined mass-to-charge ratio is counted. Typically, an equivalent precursor ion bearing a predefined number of stable isotope substitutions but otherwise chemically identical to the target ion is included in the method to act as a quantitative internal standard.

The term “immunoassay” refers to any method of quantitatively measuring the level of one or more biomarkers of the present invention by capturing and or detecting the presence of the target biomarker using one or more binding agents. Immunoassays may be direct, where the biomarker is adsorbed onto a surface and detected using a binding agent carrying a detectable label, indirect where the biomarker is adsorbed onto a surface, a first binding agent with specificity for the biomarker is then captured onto the surface through specific binding to the target biomarker and detected using a second binding agent carrying a detectable label that is specific for the first antibody. Alternatively the immunoassay may be a sandwich immunoassay in which a binding agent is immobilised on a solid support and captures the target biomarker from the analytical sample. Subsequently, the now immobilised biomarker is detected using binding agents in the direct or indirect methods as described above.

The term “bead suspension array” means an immunoassay where the biomarker is detected on one or mall solid particles held in a liquid suspension.

The term “planar array” means an immunoassay system where individual biomarkers or binding agents are immobilised in discrete addressable locations on a continuous solid surface including but not limited to glass slide, silicon wafer, nitrocellulose strip. Subsequent steps of an immunoassay are then performed using common reagents applied to the entire surface of the planar array or may be added individually at the appropriate addressable location within the array.

The term “isolated”, or grammatical equivalents thereof, means throughout this specification, that the protein, antibody, polynucleotide or chemical molecule as the case may be, exists in a physical milieu distinct from that in which it may occur in nature.

As used herein, the term “subject” includes any human or non-human animal. The term “non-human animal” includes all vertebrates, e.g., mammals and non-mammals, such as non-human primates, rodents, sheep, dogs, cats, horses, cows, chickens, amphibians, reptiles, etc.

The term “treat”, “treating”, “treatment”, “prevent”, “preventing” or “prevention”, or grammatical equivalents thereof, includes therapeutic treatments, prophylactic treatments and applications in which one reduces the risk that a subject will develop a disorder or other risk factor.

Treatment does not require the complete curing of a disorder and encompasses the reduction of the symptoms or underlying risk factors.

The term “diagnosis”, or grammatical equivalents thereof, as used herein, includes the provision of any information concerning the existence or presence, non-existence or absence or probability of the disorder in a patient. It further includes the provision of information concerning the type or classification of the disorder or of symptoms which are or may be experienced in connection with it. This may include, for example, diagnosis of the severity of the disorder. It encompasses prognosis of the medical course of the disorder, for example its duration, severity and the course of progression from mild cognitive impairment (MCI) to AD or other dementias.

The term “staging”, or grammatical equivalents thereof, as used herein, means identifying in a subject the stage of a neurocognitive disorder, in particular AD. For example, AD is characterised by 3 stage or 7 stages, depending on the diagnostic framework used. The Global Dementia Scale is one such measure of global function. It is measured by assessment of severity including cognition and function against a standardised set of severity criteria.

The term “efficacy” indicates the capacity for beneficial change of a given intervention (e.g. a drug, medical device, surgical procedure, etc.). If efficacy is established, that intervention is likely to be at least as good as other available interventions, to which it will have been compared. The term “efficacy” and “effectiveness” are used herein interchangeably.

The term “comprising” indicates that the subject includes all the elements listed, but may, optionally, also include additional, unnamed elements (i.e. open).

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless the context dictates otherwise, the definitions of the features/terms set out above are not limited to any particular aspect or embodiment of the invention and apply equally to all aspects and embodiments which are described herein.

### Abbreviations

CSF (cerebrospinal fluid); LBD (Lewy body dementia); FTD (fronto-temporal dementia); VaD (vascular dementia); ALS (amyotrophic lateral sclerosis) CJD (Creutzfeldt-Jakob disease); CNS (central nervous system); TMT® (Tandem Mass Tag®); TEAB (Tetra-ethylamonium Bicarbonate); TFA (Trifluoroacetic acid); SDS (Sodium dodecyl sulfate); TCEP (Tris(2-carboxyethyl)phosphine); ACN (Acetonitrile); Da (Dalton); HPLC (High-performance liquid chromatography); FA (Formic acid); IFC (Intelligent flow control); LC-MS/MS (Liquid Chromatography with tandem Mass Spectrometry detection); MS (Mass Spectrometry); MS/MS or MS2 (Tandem MS); MS/MS/MS or MS3 (triple MS) PAGE (Polyacrylamide gel electrophoresis); SCX (Strong Cation Exchange); ppm (Parts per million); TiO₂ (titanium dioxide); IMAC (iron metal affinity chromatography); ELISA (enzyme-linked immunosorbent assay).

### DETAILED DESCRIPTION

### 1. Proteins Panels and Methods of Using Thereof

Whilst it is widely accepted that tau is involved in the pathology of neurodegenerative disorders such as tauopathies, like AD, and that it actively participates in the formation of neurofibrillary tangles, not much is known about the molecular processes that mediate toxicity of over expressed and hyperphosphorylated tau.

The present inventors hypothesized that tau over expression and hyperphosphorylation causes tau toxicity in the brain leading to further events, including activation of signalling pathways, which may contribute to the advancement and severity of the disease. They further hypothesised that many of the proteins involved in these pathways would be released into blood and CSF during the evolution of tau toxicity and would be detectable early in the disease process.

The present inventors have surprisingly found that multiple cellular processes, the associated proteins and/or their levels are modified in a tau dose-dependent manner and that these affected pathways are correlated with several hallmarks of AD.

Furthermore, the panel of biomarkers identified herein are not exclusively expressed in the brain, they are surprisingly also identifiable in the cerebrospinal fluid (CSF) and most importantly their abundance in CSF is regulated between non-AD and AD patients with substantive memory effects.

Finally, the present inventors successfully demonstrated herein that upon administration of tau kinase inhibitors (enzymes which inhibits tau phosphorylation), the abundance of the proteins (i.e. biomarkers) of the panels according to the present invention increases or decreases inversely to their pre-administration abundance, indicating that the inhibitors are effective in reducing hyperphosphorylation of tau. Most surprisingly, as the abundance of these proteins increases or decreases inversely to their pre-administration abundance, also the tau toxicity comparably decreases.

The present invention provides for a panel of biomarkers comprising:
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform or a variant or a fragment thereof.

Protein phosphatase 1 regulatory subunit 14A is an inhibitor of PPP1CA (serine/threonine-protein phosphatase PP1-alpha catalytic subunit). Has over 1000-fold higher inhibitory activity when phosphorylated, creating a molecular switch for regulating the phosphorylation status of PPP1CA substrates and smooth muscle contraction.

Described to participate in RNA metabolism in the myelinating cell, 2′,3′-cyclic-nucleotide 3′-phosphodiesterase is the third most abundant protein in central nervous system myelin. Its catalytic activity is on nucleoside 2′,3′-cyclic phosphate which is transformed into nucleoside 2′-phosphate.

An isoform is described herein as an alternative protein sequence with respect to the canonical sequence. Isoforms can be generated from the same gene by a single or by the combination of alternative promoter usage, alternative splicing, alternative initiation and ribosomal frameshifting.

A variant is described herein as to include natural variants such as (naturally occurring) polymorphisms, variations between strains, isolates or cultivars, disease-associated mutations and RNA editing events. A variant is generally reported as the amino acid change with respect to the canonical sequence. Most naturally occurring polymorphisms (also called single amino acid polymorphisms or SAPs) are due to a single nucleotide change at the codon level. RNA editing events include conversion, insertion and deletion of nucleotides.

A fragment is described herein as the result of proteolytic (enzymatic or else) cleavage of a protein. Fragments may be the results of natural proteolytic cleavage for example fragments generated during the activation of complement, the clotting cascade, or from enzymatic cleavage of matrix proteins. Alternatively, fragments may be generated in-vivo and/or in-vitro for example with proteases.

In one embodiment, the variant of protein phosphatase 1 regulatory subunit 14A comprises or has the amino acid sequence of SEQ ID NO: 1 and wherein
a) serine at position 26 is replaced by phosphoserine; or
b) threonine at position 38 is replaced by phosphothreonine; or
c) serine at position 136 is replaced by phosphoserine; or
d) glycine at position 123 is replaced by arginine.

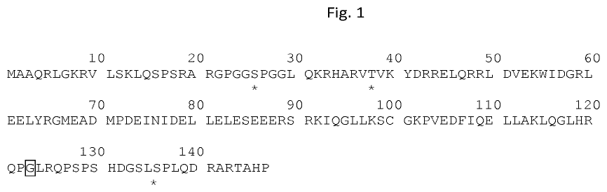

FIG. 1 shows the human sequence of protein phosphatase 1 regulatory subunit 14A (SEQ ID NO:1); flagged by symbol □ are those amino acids that are replaced with a different amino acid in isoforms of human protein phosphatase 1 regulatory subunit 14A as indicated above in d). Amino acids flagged by a * are those amino acids which are replaced by modified amino acids in isoforms of human protein phosphatase 1 regulatory subunit 14A as indicated above in a) to c).

In another embodiment the isoform of protein phosphatase 1 regulatory subunit 14A comprises or has the amino acid sequence of SEQ ID NO:1 wherein amino acid 68 to 94 are missing (isoform 2).

In another embodiment, the variant of 2′,3′-cyclic-nucleotide 3′-phosphodiesterase comprises or has the amino acid sequence of SEQ ID NO: 2 and wherein
a) tyrosine at position 110 is replaced by phosphotyrosine; or
b) cysteine at position 418 is replaced by cysteine methyl ester; or
c) cysteine at position 418 is replaced by S-farnesyl cysteine; or
d) glutamine at position 207 is replaced by arginine.

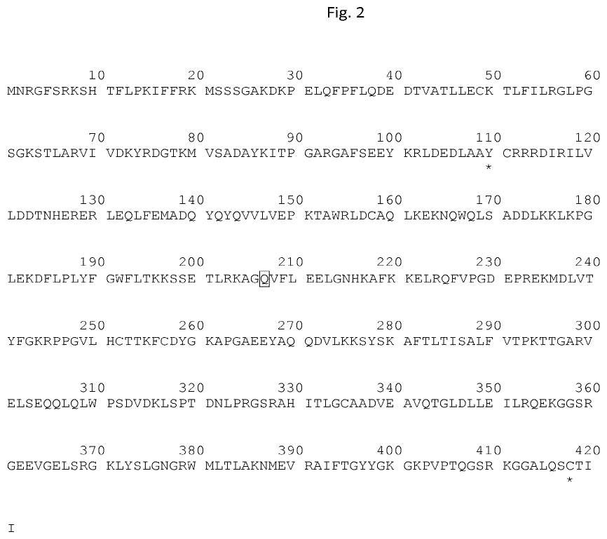

FIG. 2 shows the human sequence of 2′,3′-cyclic-nucleotide 3′-phosphodiesterase (SEQ ID NO:2); flagged by symbol □ are those amino acids that are replaced with a different amino acid in isoforms of human 2′,3′-cyclic-nucleotide 3′-phosphodiesterase as indicated above in d). Amino acids flagged by a * are those amino acids which are replaced by modified amino acids in isoforms of human protein phosphatase 1 regulatory subunit 14A as indicated above in a) to c).

In another embodiment the isoform of 2′,3′-cyclic-nucleotide 3′-phosphodiesterase comprises or has the amino acid sequence of SEQ ID NO:2 wherein amino acid 1 to 20 are missing (isoform CNPI).

In general clinical practice, proteins for use as biomarkers are measured as a set of at least 2, preferably at least 3 or 4. Hence, in addition to protein phosphatase 1 regulatory subunit 14A comprising or having the amino acid sequence of SEQ ID NO:1 or an isoform or a variant or a fragment thereof and/or 2′,3′-cyclic-nucleotide 3′-phosphodiesterase comprising or having the amino acid sequence of SEQ ID NO:2 or an isoform or a variant or a fragment thereof, the panel of biomarkers according to the present invention may further comprise one or more, alternative two or more, preferably more than two proteins selected from Group A which comprises Actin alpha cardiac muscle 1 comprising or having the amino acid sequence of SEQ ID NO:11, Antithrombin-III comprising or having the amino acid sequence of SEQ ID NO:12, BH3-interacting domain death agonist comprising or having the amino acid sequence of SEQ ID NO:3, cAMP-dependent protein kinase type I-beta regulatory subunit comprising or having the amino acid sequence of SEQ ID NO:24, Catenin delta-1 comprising or having the amino acid sequence of SEQ ID NO:4, Centrosomal protein of 170 kDa comprising or having the amino acid sequence of SEQ ID NO:23, Clathrin light chain B comprising or having the amino acid sequence of SEQ ID NO:5, Egl nine homolog 1 comprising or having the amino acid sequence of SEQ ID NO:13, Fibrinogen gamma chain comprising or having the amino acid sequence of SEQ ID NO:14, GMP reductase 1 comprising or having the amino acid sequence of SEQ ID NO:27, Guanine nucleotide-binding protein G(q) subunit alpha comprising or having the amino acid sequence of SEQ ID NO:6, Insulin-like growth factor-binding protein 6 comprising or having the amino acid sequence of SEQ ID NO:15, KxDL motif-containing protein 1 comprising or having the amino acid sequence of SEQ ID NO:28, Lambda-crystallin homolog comprising or having the amino acid sequence of SEQ ID NO:18, Myelin-associated oligodendrocyte basic protein comprising or having the amino acid sequence of SEQ ID NO:20, Neutral alpha-glucosidase AB comprising or having the amino acid sequence of SEQ ID NO:7, Nuclear pore complex protein Nup155 comprising or having the amino acid sequence of SEQ ID NO:19, OCIA domain-containing protein 1 comprising or having the amino acid sequence of SEQ ID NO:16, Protein KIAA1045 comprising or having the amino acid sequence of SEQ ID NO:25, Secernin-2 comprising or having the amino acid sequence of SEQ ID NO:8, Serum albumin comprising or having the amino acid sequence of SEQ ID NO:17, Short-chain specific acyl-CoA dehydrogenase comprising or having the amino acid sequence of SEQ ID NO:9, Synaptoporin comprising or having the amino acid sequence of SEQ ID NO:22, Syntaphilin comprising or having the amino acid sequence of SEQ ID NO:10, Transmembrane protein 119 comprising or having the amino acid sequence of SEQ ID NO: 21 and Tubulin alpha chain-like 3 comprising or having the amino acid sequence of SEQ ID NO:26.

Table 1 shows the names of the biomarkers of “Group A” and their Uniprot codes for the human protein and its mouse counterpart.

TABLE 1
Biomarkers of Group A

| SEQ | | UNIPROT | UNIPROT |
|---|---|---|---|
| ID | | ID | ID |
| NO: | Protein Name | (human) | (mouse) |
| 1 | phosphatase 1 regulatory subunit 14A | Q96A00 | Q91VC7 |
| 2 | 2′,3′-cyclic-nucleotide 3′- | P09543 | P16330 |
| | phosphodiesterase | | |
| 3 | BH3-interacting domain death agonist | P55957 | P70444 |
| 4 | Catenin delta-1 | O60716 | P30999 |
| 5 | Clathrin light chain B | P09497 | Q61RU5 |
| 6 | Guanine nucleotide-binding protein G(q) | P50148 | P21279 |
| | subunit alpha | | |
| 7 | Neutral alpha-glucosidase AB | Q14697 | Q8BHN3 |
| 8 | Secernin-2 | Q96FV2 | Q8VCA8 |
| 9 | Short-chain specific acyl-CoA | P16219 | Q07417 |
| | dehydrogenase, mitochondrial | | |
| 10 | Syntaphilin | O15079 | Q80U23 |
| 11 | Actin, alpha cardiac muscle 1 | P68032 | P68033 |
| 12 | Antithrombin-III | P01008 | P32261 |
| 13 | Egl nine homolog 1 | Q9GZT9 | Q91YE3 |
| 14 | Fibrinogen gamma chain | P02679 | Q8VCM7 |
| 15 | Insulin-like growth factor-binding protein | P24592 | P47880 |
| 16 | OCIA domain-containing protein 1 | Q9NX40 | Q9CRD0 |
| 17 | Serum albumin | P02768 | P07724 |
| 18 | Lambda-crystallin homolog | Q9Y2S2 | Q99KP3 |
| 19 | Nuclear pore complex protein Nup155 | O75694 | Q99P88 |
| 20 | Myelin-associated oligodendrocyte basic | Q13875 | Q9D2P8 |
| | protein | | |
| 21 | Transmembrane protein 119 | Q4V9L6 | Q8R138 |
| 22 | Synaptoporin | Q8TBG9 | Q8BGN8 |
| 23 | Centrosomal protein of 170 kDa | Q5SW79 | H7BX26 |
| 24 | cAMP-dependent protein kinase type 1 | P31321 | P12849 |
| 25 | Protein KIAA1045 | Q9UPV7 | Q80TL4 |
| 26 | Tubulin alpha chain-like 3 | A6NHL2 | Q3UX10 |
| 27 | GMP reductase 1 | P36959 | Q9DCZ1 |
| 28 | KxDL motif-containing protein 1 | Q9BQD3 | Q80XH1 |

In one embodiment, the panel of biomarkers according to the present invention comprises i) protein phosphatase 1 regulatory subunit 14A comprising or having the amino acid sequence of SEQ ID NO:1 or an isoform or a variant or a fragment thereof; and/or ii) 2′,3′-cyclic-nucleotide 3′-phosphodiesterase comprising or having the amino acid sequence of SEQ ID NO:2 or an isoform or a variant or a fragment thereof and one or more, preferably two or more, biomarkers selected from “Group B”, which comprises Actin alpha cardiac muscle 1 comprising or having the amino acid sequence of SEQ ID NO:11, Antithrombin-III comprising or having the amino acid sequence of SEQ ID NO:12, BH3-interacting domain death agonist comprising or having the amino acid sequence of SEQ ID NO:3, Catenin delta-1 comprising or having the amino acid sequence of SEQ ID NO:4, Clathrin light chain B comprising or having the amino acid sequence of SEQ ID NO:5, Egl nine homolog 1 comprising or having the amino acid sequence of SEQ ID NO:13, Fibrinogen gamma chain comprising or having the amino acid sequence of SEQ ID NO:14, Guanine nucleotide-binding protein G(q) subunit alpha comprising or having the amino acid sequence of SEQ ID NO:6, Insulin-like growth factor-binding protein 6 comprising or having the amino acid sequence of SEQ ID NO:15, Lambda-crystallin homolog comprising or having the amino acid sequence of SEQ ID NO:18, Neutral alpha-glucosidase AB comprising or having the amino acid sequence of SEQ ID NO:7, Nuclear pore complex protein Nup155 comprising or having the amino acid sequence of SEQ ID NO:19, OCIA domain-containing protein 1 comprising or having the amino acid sequence of SEQ ID NO:16, Secernin-2 comprising or having the amino acid sequence of SEQ ID NO:8, Serum albumin comprising or having the amino acid sequence of SEQ ID NO:17, Short-chain specific acyl-CoA dehydrogenase comprising or having the amino acid sequence of SEQ ID NO:9 and Syntaphilin comprising or having the amino acid sequence of SEQ ID NO:10.

This sub-group of biomarkers (referred herein as “Group B”) have been identified in separate in human plasma or human serum of non-AD individuals (http://www.plasmaproteomedatabase.org/). Hence, it is to be expected that these biomarkers will be not only present in blood/blood products in AD patients but also that their up/down-regulation observed in CSF will translate in the blood/blood products. This is particularly advantageous as blood and its products (plasma or serum) are more readily and easily accessible for diagnosis than CSF.

In another embodiment, the panel of biomarkers comprises i) protein phosphatase 1 regulatory subunit 14A comprising or having the amino acid sequence of SEQ ID NO:1 or an isoform or a variant or a fragment thereof; and/or ii) 2′,3′-cyclic-nucleotide 3′-phosphodiesterase comprising or having the amino acid sequence of SEQ ID NO:2 or an isoform or a variant or a fragment thereof and one or more, preferably two or more, biomarkers selected from “Group C”, which comprises Actin alpha cardiac muscle 1 comprising or having the amino acid sequence of SEQ ID NO:11, Antithrombin-III comprising or having the amino acid sequence of SEQ ID NO:12, BH3-interacting domain death agonist comprising or having the amino acid sequence of SEQ ID NO:3, cAMP-dependent protein kinase type I-beta regulatory subunit comprising or having the amino acid sequence of SEQ ID NO:24, Catenin delta-1 comprising or having the amino acid sequence of SEQ ID NO:4, Centrosomal protein of 170 kDa comprising or having the amino acid sequence of SEQ ID NO:23, Clathrin light chain B comprising or having the amino acid sequence of SEQ ID NO:5, Egl nine homolog 1 comprising or having the amino acid sequence of SEQ ID NO:13, Fibrinogen gamma chain comprising or having the amino acid sequence of SEQ ID NO:14, GMP reductase 1 comprising or having the amino acid sequence of SEQ ID NO:27, Guanine nucleotide-binding protein G(q) subunit alpha comprising or having the amino acid sequence of SEQ ID NO:6, Insulin-like growth factor-binding protein 6 comprising or having the amino acid sequence of SEQ ID NO:15, Lambda-crystallin homolog comprising or having the amino acid sequence of SEQ ID NO:18, Myelin-associated oligodendrocyte basic protein comprising or having the amino acid sequence of SEQ ID NO:20, Neutral alpha-glucosidase AB comprising or having the amino acid sequence of SEQ ID NO:7, Nuclear pore complex protein Nup155 comprising or having the amino acid sequence of SEQ ID NO:19, OCIA domain-containing protein 1 comprising or having the amino acid sequence of SEQ ID NO:16, Protein KIAA1045 comprising or having the amino acid sequence of SEQ ID NO:25, Serum albumin comprising or having the amino acid sequence of SEQ ID NO:17, Short-chain specific acyl-CoA dehydrogenase comprising or having the amino acid sequence of SEQ ID NO:9, Synaptoporin comprising or having the amino acid sequence of SEQ ID NO:22, Syntaphilin comprising or having the amino acid sequence of SEQ ID NO:10 and Tubulin alpha chain-like 3 comprising or having the amino acid sequence of SEQ ID NO:26.

This sub-group of biomarkers (which will be referred herein as “Group C”) has been found to be regulated in human CSF of AD patients versus control subjects and inversely regulated in mouse brain upon administration of a casein kinase inhibitors.

TABLE 2
Biomarkers of “Group C”.

| | | | Human CSF | | Mouse brain | |
|---|---|---|---|---|---|---|
| | | | AD/cntrl | | Log 2 | |
| SEQ | | | Log 2 | | Ck1 | |
| ID | UNIPROT | | Braak stage | | inhibitor | |
| NO: | ID | Protein Name | 3/4 | 5/6 | PS110 | PS278 |
| 1 | Q96A00 | phosphatase 1 regulatory | 0.79 | 0.69 | 0.71 | 0.49 |
| | | subunit 14A | | | | |
| 2 | P09543 | 2′,3′-cyclic-nucleotide 3′- | 0.57 | 0.57 | 0.63 | 0.62 |
| | | phosphodiesterase | | | | |
| 3 | P55957 | BH3-interacting domain death | 0.80 | — | −0.53 | −0.56 |
| | | agonist | | | | |
| 4 | O60716 | Catenin delta-1 | — | 0.58 | −2.26 | −1.07 |
| 5 | P09497 | Clathrin light chain B | 0.90 | — | 0.70 | 0.50 |
| 6 | P50148 | Guanine nucleotide-binding | 0.59 | — | 0.82 | 0.62 |
| | | protein G(q) subunit alpha | | | | |
| 7 | Q14697 | Neutral alpha-glucosidase AB | 0.72 | 0.59 | 0.52 | −0.78 |
| 9 | P16219 | Short-chain specific acyl- | −0.75 | — | 0.54 | 0.57 |
| | | CoA dehydrogenase, | | | | |
| | | mitochondrial | | | | |
| 10 | O15079 | Syntaphilin | — | 0.52 | −0.71 | −0.76 |
| 11 | P68032 | Actin, alpha cardiac muscle 1 | 0.56 | — | 0.58 | — |
| 12 | P01008 | Antithrombin-III | −0.59 | — | 0.57 | — |
| 13 | Q9GZT9 | Egl nine homolog 1 | 0.77 | — | — | −073 |
| 14 | P02679 | Fibrinogen gamma chain | −0.79 | — | 1.01 | — |
| 15 | P24592 | Insulin-like growth factor- | 0.86 | — | −0.53 | — |
| | | binding protein | | | | |
| 16 | Q9NX40 | OCIA domain-containing | 0.77 | 0.94 | — | −0.59 |
| | | protein 1 | | | | |
| 17 | P02768 | Serum albumin | −0.63 | — | 0.69 | — |
| 18 | Q9Y2S2 | Lambda-crystallin homolog | 0.60 | — | — | 0.56 |
| 19 | O75694 | Nuclear pore complex protein | 0.49 | — | −0.53 | — |
| | | Nup155 | | | | |
| 20 | Q13875 | Myelin-associated | 0.60 | — | 0.84 | 0.66 |
| | | oligodendrocyte basic protein | | | | |
| 22 | Q8TBG9 | Synaptoporin | 0.91 | 1.03 | — | 0.72 |
| 23 | Q5SW79 | Centrosomal protein of 170 kDa | 0.91 | 0.92 | — | −0.51 |
| 24 | P31321 | cAMP-dependent protein kinase | 1.56 | — | 0.53 | — |
| | | type 1 | | | | |
| 25 | Q9UPV7 | Protein KIAA1045 | 0.67 | — | −0.63 | — |
| 26 | A6NHL2 | Tubulin alpha chain-like 3 | 1.75 | — | — | 0.67 |
| 27 | P36959 | GMP reductase 1 | 1.30 | — | 0.68 | — |

In another embodiment the panel of biomarkers according to the present invention comprises i) protein phosphatase 1 regulatory subunit 14A comprising or having the amino acid sequence of SEQ ID NO:1 or an isoform or a variant or a fragment thereof; and/or ii) 2′,3′-cyclic-nucleotide 3′-phosphodiesterase comprising or having the amino acid sequence of SEQ ID NO:2 or an isoform or a variant or a fragment thereof and one or more, preferably two or more, biomarkers selected from “Group D”, which comprises Secernin-2 comprising or having the amino acid sequence of SEQ ID NO:8, Transmembrane protein 119 comprising or having the amino acid sequence of SEQ ID NO: 21 KxDL motif-containing protein 1 comprising or having the amino acid sequence of SEQ ID NO:28.

This sub-group of biomarkers (which will be referred herein as “Group D”) has been found to be regulated in the brain of AD patients versus control and regulated in mouse brain upon administration of casein kinase inhibitors.

TABLE 3
Biomarkers of “Group D”.

| | | | Human | Mouse brain | |
|---|---|---|---|---|---|
| SEQ | | | brain | Log 2 | |
| ID | UNIPROT | | AD/cntrl | Ck1 inhibitor | |
| NO: | ID | Protein Name | Log 2 | PS110 | PS278 |
| 1 | Q96A00 | phosphatase 1 regu- | 0.79 | 0.69 | 0.71 |
| | | latory subunit 14A | | | |
| 2 | P09543 | 2′,3′-cyclic-nucleotide | 0.57 | 0.57 | 0.63 |
| | | 3′-phosphodiesterase | | | |
| 8 | Q96FV2 | Secernin-2 | 0.56 | −0.76 | −0.52 |
| 21 | Q4V9L6 | Transmembrane | 1.15 | −0.66 | −0.52 |
| | | protein 119 | | | |
| 28 | Q9BQD3 | KxDL motif-con- | 0.58 | −0.58 | — |
| | | taining protein 1 | | | |

Groups C and D (listed in Tables 2 and 3, respectively) comprise biomarkers which have been shown to be regulated in mouse brain following administration of two different casein kinase inhibitors and i) (Table 2; Group C) biomarkers which are up or down-regulated in the CSF of human patient with AD at Braak stages 3/4 or 5/6 or ii) (Table 3; Group D) biomarkers which are up/down-regulated in human brain of AD patients versus control subjects. These biomarkers are very advantageous as they allow to diagnose and stage the tauopathy but also serve as pharmacodynamics biomarkers allowing to establish if a treatment is effective in that specific patient or if an alternative approach should be followed.

Furthermore, the present invention provides for a panel of biomarkers comprising tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422;
   wherein when the phosphorylated amino acid on tau is T181, the panel comprises tau or one or more fragments thereof having at least one more phosphorylated amino acid.

The microtubule-associated protein tau becomes abnormally phosphorylated in the hippocampus and cortex of patients with Alzheimer's disease, ultimately forming aggregates which organise into paired helical filaments (PHF tau), a pathological hallmark of the condition. Certain phosphorylated forms of tau protein can propagate through the brain by trans-synaptic spreading and presumably through this and other poorly understood processes is also found in cerebrospinal fluid. The ratio of tau phosphorylated at serine 181 (based on 2N4R tau isoform sequence; SEQ ID NO: 29) relative to total tau within the CSF is an accepted biomarker used to classify individuals as having pre-symptomatic Alzheimer's disease and to confirm the clinical diagnosis in symptomatic disease.

The amino acids phosphorylated on tau having or comprising the amino acid sequence of SEQ ID NO: 29 or fragments thereof identified in the present inventions are serine and/or threonine and/or tyrosine amino acids as illustrated in Table 4.

TABLE 4
Amino acids of tau protein (SEQ ID NO: 29) found
to be phosphorylated in tau from human CSF and/or
human brain and/or mouse brain (X means detected)

| Amino acid Number | Mouse Brain | Human Brain | Human CSF |
|---|---|---|---|
| T39 | X | | |
| S46 | X | X | X |
| T50 | X | X | |
| T52 | X | | |
| T56 | X | X | |
| S61 | X | X | X |
| T63 | X | | |
| S64 | X | | X |
| S68 | X | | |
| T69 | X | | |
| S113 | X | | |
| T181 | X | X | X |
| S184 | X | X | X |
| S185 | X | X | X |
| S191 | X | X | |
| S195 | X | | |
| S198 | X | X | X |
| S199 | X | X | X |
| S202 | X | X | X |
| S205 | X | X | X |
| S208 | X | X | X |
| S210 | X | X | |
| T212 | X | X | X |
| S214 | X | X | X |
| T217 | X | X | X |
| T231 | X | X | X |
| S235 | | X | X |
| S237 | | X | X |
| S238 | | X | X |
| S258 | | X | X |
| S262 | X | X | X |
| S285 | | | X |
| S289 | | X | X |
| S356 | X | X | X |
| Y394 | X | X | |
| S396 | X | X | X |
| S400 | X | X | X |
| T403 | X | X | X |
| S404 | | X | X |
| S409 | X | X | |
| S412 | X | | |
| S413 | X | | |
| T414/S416 | X | | |
| S422 | | X | |

Each of the amino acid shown in Table 4 have diagnostic, prognostic and/or pharmacodynamic biomarker utility. Preferably the seventeen phosphorylated residues that were detected in all studies (X in all columns) have greatest potential as pharmacodynamics biomarkers as they are present during human disease, are also detected in an animal model treated with a casein kinase inhibitor, and could also be detected in CSF of humans.

More preferably, the panel of biomarkers comprising tau or one or more fragments thereof, wherein tau:
i) comprises or has the amino acid sequence of SEQ ID NO:29 and
ii) comprises phosphorylated amino acids S61, S64, T181, S184, S202, S205, T231 and/or S235.

In one embodiment, the panel of biomarkers comprising tau or one or more fragments thereof, wherein tau:
i) comprises or has the amino acid sequence of SEQ ID NO:29 and
ii) comprises one or more, optionally two or more phosphorylated amino acids selected from S61, S64, S199, S205, and S396.

In another embodiment, the panel of biomarkers comprising tau or one or more fragments thereof, wherein tau:
i) comprises or has the amino acid sequence of SEQ ID NO:29 and
ii) comprises phosphorylated amino acid S61.

In another embodiment, the panel of biomarkers comprising tau or one or more fragments thereof, wherein tau:
i) comprises or has the amino acid sequence of SEQ ID NO:29 and ii) comprises phosphorylated amino acid S64.

In another embodiment, the panel of biomarkers comprising tau or one or more fragments thereof, wherein tau:
i) comprises or has the amino acid sequence of SEQ ID NO:29 and
ii) comprises phosphorylated amino acid S199.

In another embodiment, the panel of biomarkers comprising tau or one or more fragments thereof, wherein tau:
i) comprises or has the amino acid sequence of SEQ ID NO:29 and
ii) comprises phosphorylated amino acid S205.

In another embodiment, the panel of biomarkers comprising tau or one or more fragments thereof, wherein tau:
i) comprises or has the amino acid sequence of SEQ ID NO:29 and
ii) comprises phosphorylated amino acid S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61 and S64.

In another embodiment, the panel of biomarkers comprising tau or one or more fragments thereof, wherein tau:
i) comprises or has the amino acid sequence of SEQ ID NO:29 and
ii) comprises phosphorylated amino acids S61 and S199.

In another embodiment, the panel of biomarkers comprising tau or one or more fragments thereof, wherein tau:
i) comprises or has the amino acid sequence of SEQ ID NO:29 and
ii) comprises phosphorylated amino acids S61 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S61 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S64 and S199.

In another embodiment, the panel of biomarkers comprising tau or one or more fragments thereof, wherein tau:
i) comprises or has the amino acid sequence of SEQ ID NO:29 and
ii) comprises phosphorylated amino acids S64 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S64 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S199 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S199 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S205 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64 and S199.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S199 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S199 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S205 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S64, S199 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S64, S199 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S64, S205 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S199, S205 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64, S199 and S205.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64, S199 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S199, S205 and S396.

In another embodiment, the panel of biomarkers comprising tau or one or more fragments thereof, wherein tau:
i) comprises or has the amino acid sequence of SEQ ID NO:29 and
ii) comprises phosphorylated amino acids S61, S64, S199, S205, and S396.

The embodiments relating to the amino acid sequence and the particular phosphorylated amino acids in tau or on one or more fragments thereof, as described above, are equally applicable to all other embodiments of the second aspect of the invention and to all other aspects of the invention where tau or on one or more fragments thereof is involved.

In one embodiment, the panel of biomarker comprises at least one biomarker selected from Groups A, B, C or D and one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422;
   wherein when the phosphorylated amino acid on tau is T181, the panel comprises tau or one or more fragments thereof having at least one more phosphorylated amino acid. Preferably, the biomarker selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

More preferably, the panel comprises three or more, four or more, five or more biomarkers, wherein at least two biomarkers, in addition to tau, are selected from Groups A, B, C or D and are:
i) protein phosphatase 1 regulatory subunit 14A comprising or having the amino acid sequence of SEQ ID NO:1 or an isoform or a variant or a fragment thereof;
and
ii) 2,3′-cyclic-nucleotide 3′-phosphodiesterase comprising or having the amino acid sequence of SEQ ID NO:2 or an isoform or a variant or a fragment thereof.

Additionally, the present invention provides for a panel of biomarkers comprising one or more, optionally two or more proteins selected from Tables 5, 6, 7, 8, 9, 10, 11, 12, 13 or combinations thereof.

These biomarkers have been found to be highly regulated in the CSF of AD patients versus control. All non-modified peptides for each protein were summed for three control and three AD cases. The log 2 ratio and p-value of each protein was then calculated. Proteins with 2 or more peptides, >60% regulation and p<0.05 were selected as biomarkers for Table 5.

TABLE 5
Biomarkers up/down-regulated in the
CSF of AD patient versus control

| | | Log2 | |
|---|---|---|---|
| UniProtKB | | AD/ | |
| Accession | | Con- | P |
| Number | Protein name | trol | value |
| I3L192 | Basigin | −3.86 | 0.04 |
| E5RJZ1 | Cytochrome c oxidase subunit 7A- | 4.19 | 0.05 |
| | related protein, mitochondrial | | |
| K7EIT4 | 14-3-3 protein epsilon | 0.78 | 0.04 |
| K7EJ68 | 3-ketoacyl-CoA thiolase, | 0.91 | 0.01 |
| | mitochondrial | | |
| B4DVF4 | 3-ketoacyl-CoA thiolase, peroxisomal | 1.28 | 0.02 |
| P25325 | 3-mercaptopyruvate sulfurtransferase | 0.81 | 0.01 |
| P08708 | 40S ribosomal protein S17 | 1.34 | 0.01 |
| E5RIP1 | 40S ribosomal protein S20 | 1.32 | 0.01 |
| D6R9I7 | 40S ribosomal protein S23 | 0.83 | 0.04 |
| P61247 | 40S ribosomal protein S3a | 0.96 | 0.04 |
| F5GZI0 | 4F2 cell-surface antigen heavy chain | 1.29 | 0.03 |
| P32754 | 4-hydroxyphenylpyruvate dioxygenase | 0.76 | 0.03 |
| F8VPE8 | 60S acidic ribosomal protein P0 | 0.73 | 0.03 |
| H7C3M2 | 60S ribosomal protein L3 | 2.45 | 0.01 |
| C9JIJ5 | 60S ribosomal protein L7 | 0.86 | <0.01 |
| Q8IZP0 | Abl interactor 1 | 1.04 | 0.01 |
| H0YN26 | Acidic leucine-rich nuclear | 1.76 | 0.04 |
| | phosphoprotein 32 family member A | | |
| E9PF58 | Actin-related protein 2/3 complex | 0.94 | 0.02 |
| | subunit 1A | | |
| B4DXW1 | Actin-related protein 3 | 0.73 | 0.04 |
| I3L1U8 | Active breakpoint cluster region- | 0.92 | <0.01 |
| | related protein | | |
| O14561 | Acyl carrier protein, mitochondrial | 0.72 | 0.02 |
| P07108 | Acyl-CoA-binding protein | 1.18 | 0.02 |
| O14734 | Acyl-coenzyme A thioesterase 8 | 0.76 | <0.01 |
| E9PQQ8 | Adenylate kinase isoenzyme 5 | 0.89 | 0.02 |
| P12235 | ADP/ATP translocase 1 | 0.71 | 0.03 |
| F5H1V1 | ADP-ribosylation factor 3 | 0.74 | 0.03 |
| Q9NVJ2 | ADP-ribosylation factor-like protein | 0.94 | 0.01 |
| | 8B | | |
| H0Y5U1 | Agrin | 1.05 | 0.03 |
| P11766 | Alcohol dehydrogenase class-3 | 0.87 | 0.05 |
| A6NHU4 | Aldo-keto reductase family 1 member | −1.04 | 0.01 |
| | C1 | | |
| P55008 | Allograft inflammatory factor 1 | 0.82 | 0.03 |
| P02763 | Alpha-1-acid glycoprotein 1 | −1.03 | 0.02 |
| G3V3A0 | Alpha-1-antichymotrypsin | −0.84 | 0.04 |
| H0YJ11 | Alpha-actinin-1 | 0.67 | 0.01 |
| M0R2M1 | Alpha-soluble NSF attachment protein | 0.74 | 0.03 |
| P05067 | Amyloid beta A4 protein | 0.90 | <0.01 |
| K7EMN4 | Amyloid-like protein 1 | 0.73 | 0.01 |
| E9PQS3 | Amyloid-like protein 2 | 0.94 | 0.01 |
| P03950 | Angiogenin | −0.76 | 0.03 |
| E7EV01 | Ankyrin repeat and SOCS box protein 2 | 1.68 | 0.01 |
| P16157 | Ankyrin-1 | 0.86 | 0.01 |
| D6R9U4 | Ankyrin-2 | 0.65 | 0.03 |
| V9GYC1 | Apolipoprotein A-II | −0.85 | <0.01 |
| P06727 | Apolipoprotein A-IV | −1.97 | 0.04 |
| P04114 | Apolipoprotein B-100 | 0.70 | 0.04 |
| B0YIW2 | Apolipoprotein C-III | −0.77 | 0.04 |
| G3V1B6 | Apolipoprotein O | 0.84 | 0.03 |
| H0YER7 | Apoptosis inhibitor 5 | 0.79 | 0.04 |
| E9PMA0 | Apoptosis-inducing factor 1, | 0.69 | 0.03 |
| | mitochondrial | | |
| P29972 | Aquaporin-1 | 0.90 | <0.01 |
| B0YIW6 | Archain 1 | −1.26 | 0.01 |
| F8VXI9 | ARF GTPase-activating protein GIT2 | 1.10 | 0.02 |
| Q96P47 | Arf-GAP with GTPase, ANK repeat and | 0.66 | 0.01 |
| | PH domain-containing protein 3 | | |
| O43776 | Asparagine-tRNA ligase, cytoplasmic | 1.37 | 0.03 |
| P17174 | Aspartate aminotransferase, | 0.99 | 0.03 |
| | cytoplasmic | | |
| B1AJS1 | Astrotactin-1 | 0.70 | 0.01 |
| K7EJP1 | ATP synthase subunit alpha, | 0.77 | 0.04 |
| | mitochondrial | | |
| C9JU26 | ATP synthase subunit f, mitochondrial | 0.75 | 0.03 |
| P17858 | ATP-dependent 6-phosphofructokinase, | 0.68 | 0.02 |
| | liver type | | |
| Q96BJ3 | Axin interactor, dorsalization- | 0.98 | 0.04 |
| | associated protein | | |
| Q4VXN1 | Band 4.1-like protein 1 | 1.36 | 0.01 |
| E9PQD2 | Band 4.1-like protein 2 | 3.48 | 0.02 |
| P02749 | Beta-2-glycoprotein 1 | −0.70 | 0.00 |
| Q562R1 | Beta-actin-like protein 2 | 0.77 | 0.05 |
| P35612 | Beta-adducin | 0.77 | 0.05 |
| Q96KN2 | Beta-Ala-His dipeptidase | 0.71 | 0.05 |
| P07686 | Beta-hexosaminidase subunit beta | 1.49 | 0.00 |
| C9JSN9 | Biotinidase | 0.97 | 0.01 |
| Q9NZE6 | Eukaryotic translation initiation | 0.75 | 0.01 |
| | factor 4A, isoform 2, isoform CRA_b | | |
| Q8WXS3 | Brain and acute leukemia cytoplasmic | 0.88 | 0.05 |
| | protein | | |
| P11274 | Breakpoint cluster region protein | 0.99 | 0.01 |
| Q96GW7 | Brevican core protein | 0.69 | 0.03 |
| Q9HCU4 | Cadherin EGF LAG seven-pass G-type | 0.68 | <0.01 |
| | receptor 2 | | |
| P19022 | Cadherin-2 | 1.17 | 0.05 |
| Q9UQM7 | Calcium/calmodulin-dependent protein | 0.81 | 0.02 |
| | kinase type II subunit alpha | | |
| E7ETC9 | Calcium/calmodulin-dependent protein | 1.08 | <0.01 |
| | kinase type II subunit beta | | |
| Q16566 | Calcium/calmodulin-dependent protein | 0.85 | 0.03 |
| | kinase type IV | | |
| H7C4P2 | Calcium-dependent secretion activator | 0.87 | 0.03 |
| | 1 | | |
| F8WBR5 | Calmodulin | 1.09 | 0.03 |
| Q9P1Y5 | Calmodulin-regulated spectrin- | 0.91 | 0.01 |
| | associated protein 3 | | |
| P07384 | Calpain-1 catalytic subunit | 1.06 | 0.03 |
| O94985 | Calsyntenin-1 | 0.75 | 0.01 |
| Q9BQT9 | Calsyntenin-3 | 0.76 | 0.01 |
| O43852 | Calumenin | 1.15 | 0.01 |
| C9J9E2 | CaM kinase-like vesicle-associated | 0.70 | 0.05 |
| | protein | | |
| K7EM13 | cAMP-dependent protein kinase type I- | 0.97 | <0.01 |
| | alpha regulatory subunit | | |
| P31321 | cAMP-dependent protein kinase type I- | 1.56 | 0.04 |
| | beta regulatory subunit | | |
| Q5TDF0 | Cancer-related nucleoside- | 0.93 | 0.01 |
| | triphosphatase | | |
| Q8WXD9 | Caskin-1 | 0.76 | 0.04 |
| Q92851 | Caspase-10 | 1.21 | 0.01 |
| Q86VU5 | Catechol O-methyltransferase domain- | 1.06 | 0.01 |
| | containing protein 1 | | |
| C9IZ88 | Catenin alpha-2 | 2.11 | 0.01 |
| E9PKT6 | Cathepsin H | 0.73 | 0.01 |
| E9PNW4 | CD59 glycoprotein | 0.79 | 0.04 |
| Q5JYX0 | Cell division control protein 42 | 1.04 | 0.05 |
| | homolog | | |
| Q99674 | Cell growth regulator with EF hand | 0.86 | <0.01 |
| | domain protein 1 | | |
| Q5SW79 | Centrosomal protein of 170 kDa | 0.92 | 0.02 |
| Q8NI60 | Chaperone activity of bc1 complex- | 0.78 | 0.01 |
| | like, mitochondrial | | |
| Q7LBR1 | Charged multivesicular body protein | 0.66 | 0.01 |
| | 1b | | |
| Q96FZ7 | Charged multivesicular body protein 6 | 0.83 | <0.01 |
| Q9BWS9 | Chitinase domain-containing protein 1 | 0.85 | 0.02 |
| J3KS05 | Chromobox protein homolog 1 | 0.84 | 0.01 |
| G5E968 | Chromogranin A, isoform CRA_b | 0.78 | 0.01 |
| Q9UPT6 | C-Jun-amino-terminal kinase- | −0.85 | 0.01 |
| | interacting protein 3 | | |
| Q8IZR5 | CKLF-like MARVEL transmembrane | 0.70 | 0.01 |
| | domain-containing protein 4 | | |
| E5RGY9 | Clathrin coat assembly protein AP180 | 1.45 | 0.02 |
| D6RJD1 | Clathrin light chain B | 0.84 | 0.02 |
| Q14019 | Coactosin-like protein | 0.68 | 0.02 |
| P00740 | Coagulation factor IX | −0.73 | 0.05 |
| P53618 | Coatomer subunit beta | 1.07 | 0.03 |
| H0Y8X7 | Coatomer subunit gamma-1 | 0.69 | 0.03 |
| I3L0M4 | Coiled-coil domain-containing protein | 0.99 | 0.01 |
| | 43 | | |
| Q96A33 | Coiled-coil domain-containing protein | 0.79 | 0.02 |
| | 47 | | |
| H7C5H1 | Complement factor B | −1.11 | 0.03 |
| Q03591 | Complement factor H-related protein 1 | −0.67 | <0.01 |
| V9GYE7 | Complement factor H-related protein 2 | −1.01 | 0.01 |
| E9PDN6 | Contactin-associated protein-like 4 | 0.85 | 0.01 |
| H0YKU5 | COP9 signalosome complex subunit 2 | 1.31 | 0.01 |
| Q92905 | COP9 signalosome complex subunit 5 | 0.76 | 0.01 |
| O75367 | Core histone macro-H2A.1 | 0.67 | 0.01 |
| H0YJG0 | Creatine kinase B-type | 0.68 | 0.03 |
| E7EPF8 | C-terminal-binding protein 1 | 0.84 | 0.01 |
| E9PC62 | CUGBP Elav-like family member 2 | 0.99 | 0.01 |
| F5H6I6 | Cullin-associated NEDD8-dissociated | 1.06 | 0.04 |
| | protein 1 | | |
| E9PHZ2 | Cysteine and histidine-rich domain- | 0.67 | 0.02 |
| | containing protein 1 | | |
| Q16878 | Cysteine dioxygenase type 1 | 1.32 | 0.05 |
| H0YFA4 | Cysteine-rich protein 2 | 0.95 | 0.03 |
| H3BP04 | Cytochrome b-c1 complex subunit 2, | 0.67 | 0.02 |
| | mitochondrial | | |
| P14927 | Cytochrome b-c1 complex subunit 7 | 0.98 | 0.05 |
| P47985 | Cytochrome b-c1 complex subunit | 1.13 | 0.04 |
| | Rieske, mitochondrial | | |
| Q5JTJ3 | Cytochrome c oxidase assembly factor | 1.23 | 0.01 |
| | 6 homolog | | |
| K7EQD3 | Cytochrome c oxidase subunit 6B1 | 0.88 | 0.02 |
| Q99418 | Cytohesin-2 | 0.77 | 0.03 |
| E7EPF5 | Cytoplasmic protein NCK2 | 0.72 | 0.02 |
| C9JM78 | D-beta-hydroxybutyrate dehydrogenase, | 1.46 | 0.00 |
| | mitochondrial | | |
| Q9UKG1 | DCC-interacting protein 13-alpha | 0.79 | 0.01 |
| H7C342 | D-dopachrome decarboxylase | 0.88 | 0.03 |
| Q8NFT8 | Delta and Notch-like epidermal growth | 1.02 | 0.01 |
| | factor-related receptor | | |
| Q08495 | Dematin | 0.76 | 0.01 |
| P60981 | Destrin | 0.93 | 0.05 |
| Q9BPU6 | Dihydropyrimidinase-related protein 5 | 0.82 | 0.03 |
| E7EPF1 | Disintegrin and metalloproteinase | 1.05 | 0.01 |
| | domain-containing protein 22 | | |
| B4E2H8 | Disks large homolog 1 | 1.37 | 0.03 |
| A8MVA8 | Disks large homolog 2 | 0.72 | 0.02 |
| O00273 | DNA fragmentation factor subunit | −0.67 | 0.03 |
| | alpha | | |
| O75190 | DnaJ homolog subfamily B member 6 | 1.27 | <0.01 |
| Q9NVH1 | DnaJ homolog subfamily C member 11 | 0.94 | 0.01 |
| Q13217 | DnaJ homolog subfamily C member 3 | 0.86 | <0.01 |
| K7EIH8 | DnaJ homolog subfamily C member 7 | 0.92 | 0.02 |
| B7Z4L4 | Dolichyl-diphosphooligosaccharide- | 1.87 | 0.01 |
| | protein glycosyltransferase subunit 1 | | |
| Q9P0K9 | DOMON domain-containing protein | 1.48 | 0.01 |
| | FRRS1L | | |
| F8VYL3 | Dynamin-1-like protein | 0.89 | 0.01 |
| P50570 | Dynamin-2 | 0.89 | 0.03 |
| Q9P225 | Dynein heavy chain 2, axonemal | 1.15 | 0.00 |
| H3BS86 | E3 ubiquitin-protein ligase CHIP | 0.83 | 0.01 |
| Q05639 | Elongation factor 1-alpha 2 | 0.68 | 0.04 |
| H3BNU3 | Elongation factor Tu, mitochondrial | 1.14 | 0.01 |
| Q9NZ08 | Endoplasmic reticulum aminopeptidase | 1.16 | <0.01 |
| | 1 | | |
| H0YIV0 | Endoplasmin | 1.18 | 0.04 |
| B1AKC9 | Ephrin type-B receptor 2 | 1.26 | 0.02 |
| P20827 | Ephrin-A1 | 0.86 | 0.05 |
| Q92506 | Estradiol 17-beta-dehydrogenase 8 | 0.87 | 0.04 |
| Q92731 | Estrogen receptor beta | 1.73 | 0.01 |
| E7EMV8 | Eukaryotic initiation factor 4A-II | −0.98 | 0.03 |
| A6NJH9 | Eukaryotic translation initiation | 0.91 | 0.03 |
| | factor 1A, Y-chromosomal | | |
| O75821 | Eukaryotic translation initiation | 1.02 | 0.04 |
| | factor 3 subunit G | | |
| O43909 | Exostosin-like 3 | 1.03 | <0.01 |
| I3L252 | FAD-AMP lyase | 3.43 | 0.02 |
| H3BRW3 | FAD-linked sulfhydryl oxidase ALR | 1.50 | 0.01 |
| C9JPH9 | Fascin | 0.86 | 0.01 |
| G3V1D1 | Ferritin | 1.37 | 0.03 |
| Q6MZW2 | Follistatin-related protein 4 | 1.38 | <0.01 |
| H3BR68 | Fructose-bisphosphate aldolase A | 0.93 | <0.01 |
| H6UMI1 | Gamma-aminobutyric acid receptor- | 1.40 | 0.01 |
| | associated protein | | |
| H0YJU6 | Gamma-aminobutyric acid receptor | 0.78 | 0.01 |
| | subunit beta-3 | | |
| E5RGR6 | GDNF family receptor alpha-2 | 1.06 | <0.01 |
| G3V582 | Gephyrin | 1.07 | <0.01 |
| K7EMP8 | Glial fibrillary acidic protein | 0.66 | 0.03 |
| Q8TDQ7 | Glucosamine-6-phosphate isomerase 2 | −0.65 | 0.04 |
| K7EJ70 | Glucosidase 2 subunit beta | 0.80 | <0.01 |
| P42261 | Glutamate receptor 1 | 1.13 | 0.04 |
| G3V164 | Glutamate receptor 4 | 1.02 | 0.01 |
| F5H4N6 | Glutamate receptor-interacting | −0.87 | <0.01 |
| | protein 1 | | |
| H7BZD1 | Glutaminase kidney isoform, | 0.79 | 0.03 |
| | mitochondrial | | |
| O76003 | Glutaredoxin-3 | 0.90 | 0.04 |
| P07203 | Glutathione peroxidase 1 | 1.13 | 0.02 |
| Q03013 | Glutathione S-transferase Mu 4 | 1.09 | 0.04 |
| P78417 | Glutathione S-transferase omega-1 | 1.13 | 0.01 |
| H0YMX4 | Glycine amidinotransferase, | 1.67 | 0.04 |
| | mitochondrial | | |
| P23434 | Glycine cleavage system H protein, | 0.94 | 0.01 |
| | mitochondrial | | |
| H7C024 | Glypican-1 | 1.05 | <0.01 |
| Q9NZH0 | G-protein coupled receptor family C | 0.97 | 0.03 |
| | group 5 member B | | |
| P01112 | GTPase HRas | 1.09 | 0.01 |
| Q9Y2T3 | Guanine deaminase | 0.75 | 0.01 |
| B1AM21 | Guanine nucleotide-binding protein | 0.76 | 0.04 |
| | G(q) subunit alpha | | |
| P63092 | Guanine nucleotide-binding protein | 1.00 | 0.03 |
| | G(s) subunit alpha isoforms short | | |
| B7Z685 | Guanylate cyclase 1, soluble, beta 3, | −0.64 | 0.02 |
| | isoform CRA_c | | |
| B1ANH6 | Guanylate kinase | 0.69 | 0.01 |
| P00738 | Haptoglobin | −1.07 | <0.01 |
| P00739 | Haptoglobin-related protein | −1.65 | <0.01 |
| Q6PIK3 | HCG1995540, isoform CRA_b | 0.65 | 0.03 |
| D6RG00 | HCG2018358, isoform CRA_d | 0.75 | <0.01 |
| H3BQZ7 | HCG2044799 | 0.69 | 0.01 |
| K7ENF6 | Heat shock 70 kDa protein 12A | 0.86 | 0.04 |
| P08107 | Heat shock 70 kDa protein 1A/1B | 0.70 | 0.03 |
| R4GN69 | Heat shock protein 105 kDa | −0.83 | 0.01 |
| C9J3N8 | Heat shock protein beta-1 | −0.91 | 0.01 |
| Q5H962 | HECT, UBA and WWE domain containing 1 | 1.00 | 0.05 |
| Q15477 | Helicase SKI2W | 0.86 | 0.04 |
| F5GWX2 | Heme-binding protein 1 | 1.24 | <0.01 |
| P02100 | Hemoglobin subunit epsilon | 1.50 | 0.01 |
| Q6ZVN8 | Hemojuvelin | 0.87 | <0.01 |
| Q8IZP7 | Heparan-sulfate 6-O-sulfotransferase | 0.78 | 0.01 |
| | 3 | | |
| P05546 | Heparin cofactor 2 | −0.66 | 0.03 |
| Q9Y3E1 | Hepatoma-derived growth factor- | −0.67 | 0.04 |
| | related protein 3 | | |
| Q13151 | Heterogeneous nuclear | 0.90 | 0.03 |
| | ribonucleoprotein A0 | | |
| H0YB39 | Heterogeneous nuclear | 0.80 | 0.02 |
| | ribonucleoprotein H | | |
| M0QYQ7 | Heterogeneous nuclear | 0.67 | 0.02 |
| | ribonucleoprotein M | | |
| B1AR61 | Hexokinase-1 | 0.83 | 0.02 |
| D6RD60 | Histidine triad nucleotide-binding | 1.01 | 0.03 |
| | protein 1 | | |
| P16403 | Histone H1.2 | 1.33 | 0.05 |
| D6RCF2 | Histone H2A | 0.83 | 0.01 |
| U3KQK0 | Histone H2B | 0.73 | 0.03 |
| B4DEB1 | Histone H3 | 1.31 | 0.03 |
| Q6NXT2 | Histone H3.3C | 1.27 | 0.04 |
| Q16775 | Hydroxyacylglutathione hydrolase, | 0.85 | 0.04 |
| | mitochondrial | | |
| Q9Y4L1 | Hypoxia up-regulated protein 1 | 0.86 | 0.02 |
| P22304 | Iduronate 2-sulfatase | 1.30 | 0.03 |
| A6NGN9 | IgLON family member 5 | 1.24 | <0.01 |
| H0Y4R1 | Inosine-5′-monophosphate | 0.83 | 0.04 |
| | dehydrogenase 2 | | |
| H0YB38 | Inositol monophosphatase 3 | 1.12 | 0.04 |
| Q9UMF0 | Intercellular adhesion molecule 5 | 0.80 | 0.01 |
| K7EKJ9 | Interleukin enhancer-binding factor 3 | 1.57 | 0.04 |
| C9J826 | Junction plakoglobin | 2.03 | 0.04 |
| P13645 | Keratin, type I cytoskeletal 10 | 0.66 | 0.02 |
| O43790 | Keratin, type II cuticular Hb6 | 0.66 | 0.02 |
| E9PES4 | Kinesin-like protein KIF3A | 0.67 | 0.05 |
| P01042 | Kininogen-1 | −0.67 | 0.02 |
| Q04760 | Lactoylglutathione lyase | 0.71 | 0.04 |
| E9PLW6 | L-aminoadipate-semialdehyde | −1.29 | <0.01 |
| | dehydrogenase-phosphopantetheinyl | | |
| | transferase | | |
| E5RH50 | La-related protein 1 | 0.66 | <0.01 |
| F6S6P2 | Large proline-rich protein BAG6 | 0.89 | 0.03 |
| Q15334 | Lethal(2) giant larvae protein | 0.74 | 0.04 |
| | homolog 1 | | |
| Q9UIC8 | Leucine carboxyl methyltransferase 1 | 1.10 | <0.01 |
| H0YMX3 | Leucine-rich repeat and | 1.28 | <0.01 |
| | immunoglobulin-like domain-containing | | |
| | nogo receptor-interacting protein 1 | | |
| Q6F5E8 | Leucine-rich repeat-containing | −1.05 | 0.02 |
| | protein 16C | | |
| M0R2G0 | Leucine-rich repeat-containing | 0.76 | <0.01 |
| | protein 4B | | |
| G3V1D4 | Lin-7 homolog C (C. elegans), isoform | 0.72 | 0.05 |
| | CRA_b | | |
| Q5VVL7 | Lipoamide acyltransferase component | 0.84 | 0.03 |
| | of branched-chain alpha-keto acid | | |
| | dehydrogenase complex, mitochondrial | | |
| C9JXK9 | Lipoma-preferred partner | 1.08 | 0.01 |
| E9PJZ7 | Liprin-alpha-1 | 1.21 | 0.03 |
| Q13136 | Liprin-alpha-1 | 0.85 | 0.01 |
| G3V200 | Liprin-alpha-2 | 1.52 | 0.04 |
| A8MW50 | L-lactate dehydrogenase | 0.72 | 0.02 |
| F5GZQ4 | L-lactate dehydrogenase A chain | 0.68 | <0.01 |
| O95573 | Long-chain-fatty-acid-CoA ligase 3 | 0.82 | 0.01 |
| P10619 | Lysosomal protective protein | 0.70 | 0.02 |
| F8VV32 | Lysozyme C | −0.99 | 0.02 |
| Q9NZW5 | MAGUK p55 subfamily member 6 | 0.72 | 0.02 |
| A2A2V1 | Major prion protein | 1.20 | <0.01 |
| C9JF79 | Malate dehydrogenase | 1.07 | 0.04 |
| F5GX14 | Malectin | 0.75 | 0.02 |
| P49006 | MARCKS-related protein | 0.95 | 0.03 |
| B3KM87 | Matrin-3 | 0.70 | 0.02 |
| Q8N3F0 | Maturin | 0.89 | 0.02 |
| Q5HYI7 | Metaxin-3 | 1.07 | <0.01 |
| F8VSC4 | Methionine aminopeptidase 2 | 0.65 | 0.01 |
| Q13825 | Methylglutaconyl-CoA hydratase, | 1.50 | 0.03 |
| | mitochondrial | | |
| D6RCL2 | Microtubule-associated protein 1B | 0.73 | 0.04 |
| M0QXQ9 | Microtubule-associated protein 1S | 0.76 | 0.02 |
| Q9H936 | Mitochondrial glutamate carrier 1 | 0.66 | <0.01 |
| G3V502 | Mitochondrial import inner membrane | 0.92 | 0.03 |
| | translocase subunit Tim9 | | |
| E5RJK1 | Mitochondrial peptide methionine | 0.87 | <0.01 |
| | sulfoxide reductase | | |
| Q10713 | Mitochondrial-processing peptidase | 0.70 | 0.04 |
| | subunit alpha | | |
| D6RAU3 | Mitogen-activated protein kinase 10 | 0.65 | 0.04 |
| Q15746 | Myosin light chain kinase, smooth | 1.08 | 0.01 |
| | muscle | | |
| E7ERA5 | Myosin-10 | 0.77 | 0.02 |
| P58546 | Myotrophin | 0.79 | 0.04 |
| O95865 | N(G),N(G)-dimethylarginine | 0.83 | 0.01 |
| | dimethylaminohydrolase 2 | | |
| O14745 | Na(+)/H(+) exchange regulatory | 1.06 | 0.05 |
| | cofactor NHE-RF1 | | |
| Q4G0N4 | NAD kinase 2, mitochondrial | 0.96 | <0.01 |
| D6RAI5 | NAD(P) transhydrogenase, | 0.86 | <0.01 |
| | mitochondrial | | |
| F8VRD8 | NADH dehydrogenase [ubiquinone] 1 | 1.00 | 0.05 |
| | alpha subcomplex subunit 12 | | |
| O95182 | NADH dehydrogenase [ubiquinone] 1 | 0.73 | 0.02 |
| | alpha subcomplex subunit 7 | | |
| Q16795 | NADH dehydrogenase [ubiquinone] 1 | 0.74 | 0.04 |
| | alpha subcomplex subunit 9, | | |
| | mitochondrial | | |
| E9PQ68 | NADH dehydrogenase [ubiquinone] 1 | −0.85 | <0.01 |
| | beta subcomplex subunit 8, | | |
| | mitochondrial | | |
| E7EPT4 | NADH dehydrogenase [ubiquinone] | 0.92 | 0.05 |
| | flavoprotein 2, mitochondrial | | |
| P56181 | NADH dehydrogenase [ubiquinone] | 0.70 | 0.03 |
| | flavoprotein 3, mitochondrial | | |
| Q9UHQ9 | NADH-cytochrome b5 reductase 1 | 0.72 | 0.02 |
| Q9BXJ9 | N-alpha-acetyltransferase 15, NatA | 0.95 | 0.04 |
| | auxiliary subunit | | |
| Q8TBC4 | NEDD8-activating enzyme E1 catalytic | 0.82 | 0.01 |
| | subunit | | |
| Q59FP8 | Neogenin | 0.98 | 0.04 |
| O00533 | Neural cell adhesion molecule L1-like | 0.80 | <0.01 |
| | protein | | |
| E7EQN4 | Neurexin-1-beta | 0.99 | <0.01 |
| H7C2R8 | Neurexin-2 | 1.11 | <0.01 |
| Q9Y4C0 | Neurexin-3 | 0.93 | <0.01 |
| Q9HDB5 | Neurexin-3-beta | 0.98 | <0.01 |
| Q9NPD7 | Neuritin | 0.94 | 0.03 |
| P61601 | Neurocalcin-delta | 1.33 | 0.03 |
| Q9UBB6 | Neurochondrin | 0.80 | 0.02 |
| P16519 | Neuroendocrine convertase 2 | 1.52 | <0.01 |
| Q8NFZ4 | Neuroligin-2 | 0.96 | 0.04 |
| O95502 | Neuronal pentraxin receptor | 1.09 | <0.01 |
| P47972 | Neuronal pentraxin-2 | 1.02 | 0.01 |
| O15240 | Neurosecretory protein VGF | 1.12 | <0.01 |
| C9JQU8 | Neuroserpin | 0.71 | 0.03 |
| F5H810 | Noelin | 0.90 | <0.01 |
| H7C367 | Non-POU domain-containing octamer- | 1.02 | 0.03 |
| | binding protein | | |
| E9PLD1 | Non-specific lipid-transfer protein | 1.54 | 0.02 |
| H0YFY6 | Nuclear mitotic apparatus protein 1 | 1.10 | 0.02 |
| Q02818 | Nucleobindin-1 | 0.87 | 0.02 |
| Q86U38 | Nucleolar protein 9 | 0.86 | 0.03 |
| E5RHP0 | Nucleoside diphosphate kinase A | 0.84 | 0.05 |
| F8W543 | Nucleosome assembly protein 1-like 1 | 0.75 | 0.02 |
| P23515 | Oligodendrocyte-myelin glycoprotein | 0.80 | <0.01 |
| X6RKL2 | Optineurin | 0.81 | <0.01 |
| D6R9C5 | Osteopontin | 0.75 | 0.04 |
| Q9BZF1 | Oxysterol-binding protein-related | 0.78 | 0.04 |
| | protein 8 | | |
| Q96HC4 | PDZ and LIM domain protein 5 | 0.75 | 0.02 |
| Q9UBV8 | Peflin | 0.98 | 0.03 |
| Q02790 | Peptidyl-prolyl cis-trans isomerase | 0.73 | 0.04 |
| | FKBP4 | | |
| O14936 | Peripheral plasma membrane protein | 1.07 | 0.04 |
| | CASK | | |
| H7C5W5 | Peripherin | 1.14 | 0.04 |
| P32119 | Peroxiredoxin-2 | 0.96 | 0.03 |
| I3L0T4 | Peroxisomal acyl-coenzyme A oxidase 1 | 1.02 | 0.01 |
| Q9Y285 | Phenylalanine-tRNA ligase alpha | 0.72 | 0.02 |
| | subunit | | |
| F8VVM2 | Phosphate carrier protein, | 0.78 | 0.01 |
| | mitochondrial | | |
| A8MYT4 | Phosphatidylinositol 3-kinase | 0.83 | 0.03 |
| A8MTF1 | Phosphatidylinositol 4-kinase alpha | 0.71 | 0.01 |
| P15259 | Phosphoglycerate mutase 2 | 1.12 | 0.04 |
| A6NDG6 | Phosphoglycolate phosphatase | 1.05 | 0.03 |
| M0QZI4 | Phospholipase D3 | 0.83 | 0.01 |
| P36969 | Phospholipid hydroperoxide | 0.75 | 0.02 |
| | glutathione peroxidase, mitochondrial | | |
| Q9H008 | Phospholysine phosphohistidine | 0.74 | 0.03 |
| | inorganic pyrophosphate phosphatase | | |
| Q5SRE7 | Phytanoyl-CoA dioxygenase domain- | 0.65 | 0.01 |
| | containing protein 1 | | |
| Q9GZP4 | PITH domain-containing protein 1 | 0.71 | 0.04 |
| Q504U3 | PKM2 protein | 0.73 | 0.02 |
| I3L495 | Platelet-activating factor | 0.97 | 0.02 |
| | acetylhydrolase IB subunit alpha | | |
| Q09470 | Potassium voltage-gated channel | 0.73 | 0.04 |
| | subfamily A member 1 | | |
| Q9UHV9 | Prefoldin subunit 2 | 0.72 | 0.04 |
| F8W8W4 | Prenylcysteine oxidase 1 | 0.74 | <0.01 |
| Q8TBB6 | Probable cationic amino acid | 0.66 | 0.05 |
| | transporter | | |
| P01303 | Pro-neuropeptide Y | 0.86 | 0.04 |
| Q9H7Z7 | Prostaglandin E synthase 2 | 0.98 | 0.02 |
| Q16186 | Proteasomal ubiquitin receptor ADRM1 | 1.29 | 0.05 |
| F5GX11 | Proteasome subunit alpha type-1 | 0.84 | 0.03 |
| P28066 | Proteasome subunit alpha type-5 | 1.08 | 0.03 |
| H0Y586 | Proteasome subunit alpha type-7 | 0.78 | 0.03 |
| P02760 | Protein AMBP | −0.95 | 0.04 |
| O60678 | Protein arginine N-methyltransferase | 1.27 | 0.01 |
| | 3 | | |
| Q5TA58 | Protein argonaute | −0.78 | 0.03 |
| E9PGA6 | Protein C1QTNF3-AMACR | 0.76 | 0.01 |
| D6RAV0 | Protein CDV3 homolog | 0.69 | 0.01 |
| B4DFG0 | Protein DEK | 0.84 | 0.01 |
| I3L3P5 | Protein disulfide-isomerase | 0.86 | 0.01 |
| Q92520 | Protein FAM3C | 0.68 | 0.03 |
| Q13045 | Protein flightless-1 homolog | 0.65 | <0.01 |
| Q02156 | Protein kinase C epsilon type | 0.99 | 0.02 |
| Q99435 | Protein kinase C-binding protein | 0.68 | <0.01 |
| | NELL2 | | |
| Q5SYT8 | Protein NAMPTL | 0.83 | 0.03 |
| G3V2S0 | Protein NDRG2 | 0.94 | 0.01 |
| Q9BPW8 | Protein NipSnap homolog 1 | 0.80 | 0.04 |
| O75323 | Protein NipSnap homolog 2 | 1.61 | 0.02 |
| Q9UFN0 | Protein NipSnap homolog 3A | 0.73 | 0.04 |
| G3V3Z8 | Protein numb homolog | 0.66 | 0.01 |
| Q96A00 | Protein phosphatase 1 regulatory | 0.70 | 0.05 |
| | subunit 14A | | |
| Q9ULR3 | Protein phosphatase 1H | 0.85 | 0.01 |
| Q9Y570 | Protein phosphatase methylesterase 1 | 1.08 | 0.03 |
| Q9Y6V0 | Protein piccolo | 0.93 | 0.04 |
| E9PDC2 | Protein prune homolog 2 | 1.12 | 0.03 |
| K7EIR2 | Protein QIL1 | −0.74 | 0.03 |
| P60903 | Protein S100-A10 | 0.75 | 0.02 |
| A8MRB1 | Protein S100-B | 1.05 | 0.02 |
| H0Y8W8 | Protein transport protein Sec31A | 1.21 | <0.01 |
| H7BY58 | Protein-L-isoaspartate O- | 1.04 | 0.01 |
| | methyltransferase | | |
| Q5VT82 | Protocadherin 9 | 1.02 | <0.01 |
| Q9P2E7 | Protocadherin-10 | 0.78 | 0.01 |
| A6NEC2 | Puromycin-sensitive aminopeptidase- | 0.69 | 0.04 |
| | like protein | | |
| P0C7P4 | Putative cytochrome b-c1 complex | 1.07 | 0.03 |
| | subunit Rieske-like protein 1 | | |
| Q5VTE0 | Putative elongation factor 1-alpha- | 1.30 | 0.05 |
| | like 3 | | |
| A8MUU1 | Putative fatty acid-binding protein | 0.80 | 0.02 |
| | 5-like protein 3 | | |
| Q6DN03 | Putative histone H2B type 2-C | 1.09 | 0.02 |
| Q6GMV3 | Putative peptidyl-tRNA hydrolase | 0.67 | 0.01 |
| Q9H853 | Putative tubulin-like protein alpha- | 0.69 | 0.04 |
| | 4B | | |
| Q9NVS9 | Pyridoxine-5′-phosphate oxidase | 0.81 | 0.01 |
| H3BTN5 | Pyruvate kinase | 0.71 | 0.03 |
| E9PNP4 | Radixin | 3.05 | 0.04 |
| Q96S59 | Ran-binding protein 9 | 0.69 | 0.01 |
| Q9Y4G8 | Rap guanine nucleotide exchange | 0.72 | 0.01 |
| | factor 2 | | |
| Q13283 | Ras GTPase-activating protein-binding | 0.68 | 0.04 |
| | protein 1 | | |
| P15153 | Ras-related C3 botulinum toxin | 1.05 | 0.04 |
| | substrate 2 | | |
| P60763 | Ras-related C3 botulinum toxin | 0.97 | 0.03 |
| | substrate 3 | | |
| B4DQU5 | Ras-related protein Rab-11A | 0.67 | 0.04 |
| Q9UL25 | Ras-related protein Rab-21 | 0.69 | <0.01 |
| K7ES41 | Ras-related protein Rab-27B | 1.46 | 0.01 |
| Q8WUD1 | Ras-related protein Rab-2B | 0.80 | 0.01 |
| Q9NP90 | Ras-related protein Rab-9B | 1.47 | 0.04 |
| F6U784 | Ras-related protein Rap-2a | 1.16 | 0.05 |
| O43353 | Receptor-interacting | 0.78 | 0.04 |
| | serine/threonine-protein kinase 2 | | |
| P23471 | Receptor-type tyrosine-protein | 0.90 | <0.01 |
| | phosphatase zeta | | |
| J3KQ66 | Reelin | 0.70 | 0.01 |
| Q15493 | Regucalcin | 1.91 | 0.03 |
| H0YLG5 | Regulator of microtubule dynamics | 0.93 | 0.04 |
| | protein 3 | | |
| Q92900 | Regulator of nonsense transcripts 1 | 0.80 | 0.03 |
| B5MC59 | Replication protein A 14 kDa subunit | 0.71 | <0.01 |
| Q15293 | Reticulocalbin-1 | 1.35 | 0.03 |
| Q86UN3 | Reticulon-4 receptor-like 2 | 1.18 | 0.01 |
| Q5SYQ7 | Retinal dehydrogenase 1 | 1.04 | 0.01 |
| Q8TC12 | Retinol dehydrogenase 11 | −0.73 | 0.02 |
| J3KRE2 | Rho GDP-dissociation inhibitor 1 | 0.80 | 0.03 |
| Q9P227 | Rho GTPase-activating protein 23 | 0.88 | <0.01 |
| E9PMN0 | Ribonuclease inhibitor | 0.70 | 0.04 |
| H0YB34 | Ribonuclease UK114 | 1.04 | 0.02 |
| Q9Y3A5 | Ribosome maturation protein SBDS | 0.86 | 0.05 |
| O15034 | RIMS-binding protein 2 | 2.48 | 0.04 |
| Q5TZA2 | Rootletin | 0.77 | 0.03 |
| H7C5W9 | Sarcoplasmic/endoplasmic reticulum | 0.77 | 0.05 |
| | calcium ATPase 2 | | |
| D6RD99 | Scrapie-responsive protein 1 | 0.70 | 0.02 |
| P05060 | Secretogranin-1 | 0.80 | 0.03 |
| H0YKC2 | Secretogranin-3 | 0.87 | 0.01 |
| C9JDT0 | Secretoneurin | 1.16 | 0.01 |
| O75326 | Semaphorin-7A | 0.85 | <0.01 |
| H7C299 | Septin-5 | 0.83 | 0.02 |
| K7EJ51 | Septin-9 | 0.96 | 0.04 |
| B4DLV4 | Serine hydroxymethyltransferase | −1.13 | 0.03 |
| P34897 | Serine hydroxymethyltransferase, | −1.81 | 0.04 |
| | mitochondrial | | |
| O75494 | Serine/arginine-rich splicing factor | 0.79 | 0.02 |
| | 10 | | |
| E9PCD1 | Serine/threonine-protein kinase WNK2 | 0.75 | 0.01 |
| E9PH38 | Serine/threonine-protein phosphatase | 1.00 | 0.02 |
| | 2A 65 kDa regulatory subunit A alpha | | |
| | isoform | | |
| E9PHZ6 | Serine/threonine-protein phosphatase | 1.36 | 0.05 |
| | 2A 65 kDa regulatory subunit A beta | | |
| | isoform | | |
| Q68CR8 | Serine/threonine-protein phosphatase | 0.69 | <0.01 |
| | 2A activator | | |
| M0QWZ7 | Serine-tRNA ligase, mitochondrial | 1.17 | 0.02 |
| Q6ZV89 | SH2 domain-containing protein 5 | −0.80 | 0.04 |
| Q9BQI5 | SH3-containing GRB2-like protein 3- | 0.72 | <0.01 |
| | interacting protein 1 | | |
| P45954 | Short/branched chain specific acyl- | 1.11 | <0.01 |
| | CoA dehydrogenase, mitochondrial | | |
| A6NMU3 | Signal transducing adapter molecule 1 | 1.06 | 0.02 |
| C9K0U8 | Single-stranded DNA-binding protein, | 0.67 | 0.04 |
| | mitochondrial | | |
| Q96PX8 | SLIT and NTRK-like protein 1 | 0.77 | 0.02 |
| K7EMD6 | Small glutamine-rich | 1.62 | <0.01 |
| | tetratricopeptide repeat-containing | | |
| | protein alpha | | |
| Q8NHG7 | Small VCP/p97-interacting protein | 0.76 | 0.03 |
| Q99250 | Sodium channel protein type 2 subunit | 0.80 | 0.05 |
| | alpha | | |
| Q99884 | Sodium-dependent proline transporter | 1.26 | 0.01 |
| Q5VZ42 | Solute carrier family 12 member 5 | 0.83 | <0.01 |
| P61278 | Somatostatin | 1.03 | 0.03 |
| Q9BX66 | Sorbin and SH3 domain-containing | 0.82 | 0.05 |
| | protein 1 | | |
| Q99523 | Sortilin | 0.68 | 0.03 |
| Q9H4F8 | SPARC-related modular calcium-binding | 0.91 | <0.01 |
| | protein 1 | | |
| H0YJE6 | Spectrin beta chain, erythrocytic | 0.80 | <0.01 |
| K7EJR2 | Spermatogenesis-associated protein 22 | 0.66 | 0.03 |
| H3BS51 | Sphingomyelin phosphodiesterase 3 | 1.31 | 0.02 |
| Q13838 | Spliceosome RNA helicase DDX39B | 0.85 | 0.05 |
| H0Y9U2 | Splicing factor, proline- and | 1.45 | 0.01 |
| | glutamine-rich | | |
| Q9HCB6 | Spondin-1 | 0.75 | <0.01 |
| A2A2D0 | Stathmin | 0.78 | 0.04 |
| P31040 | Succinate dehydrogenase [ubiquinone] | 1.05 | 0.03 |
| | flavoprotein subunit, mitochondrial | | |
| C9J8Q5 | Succinate-semialdehyde dehydrogenase, | 0.77 | 0.01 |
| | mitochondrial | | |
| P17600 | Synapsin-1 | 0.95 | 0.03 |
| Q92777 | Synapsin-2 | 0.86 | 0.03 |
| Q496J9 | Synaptic vesicle glycoprotein 2C | 1.15 | 0.02 |
| K7EM19 | Synaptic vesicle membrane protein | 0.78 | 0.04 |
| | VAT-1 homolog | | |
| H7C4W3 | Synaptophysin | 2.38 | 0.03 |
| C9J0A2 | Synaptoporin | 0.92 | 0.03 |
| F5GX00 | Synaptotagmin-7 | 1.50 | <0.01 |
| F5GZI8 | T-complex protein 1 subunit alpha | 0.89 | 0.03 |
| P78371 | T-complex protein 1 subunit beta | 0.69 | 0.02 |
| E9PM09 | T-complex protein 1 subunit gamma | 1.64 | 0.03 |
| P24821 | Tenascin | 0.73 | 0.05 |
| Q08629 | Testican-1 | 1.73 | <0.01 |
| Q9BQ16 | Testican-3 | 0.84 | 0.03 |
| J3QL04 | Tether-containing UBX domain for | 1.13 | 0.03 |
| | GLUT4 | | |
| H0YB37 | Tetratricopeptide repeat protein 1 | 1.03 | 0.02 |
| Q86TV6 | Tetratricopeptide repeat protein 7B | 0.95 | <0.01 |
| P10599 | Thioredoxin | 0.93 | 0.03 |
| K7EME7 | Thioredoxin-like protein 1 | 1.28 | 0.01 |
| C9JV37 | Thrombin light chain | −0.71 | 0.03 |
| Q9Y2W1 | Thyroid hormone receptor-associated | 1.19 | 0.03 |
| | protein 3 | | |
| G3V1L9 | Tight junction protein 1 (Zona | 0.67 | 0.03 |
| | occludens 1), isoform CRA_a | | |
| P13726 | Tissue factor | 0.68 | 0.02 |
| E7EN89 | Toll interacting protein, isoform | 0.74 | 0.01 |
| | CRA_b | | |
| F2Z393 | Transaldolase | 0.86 | 0.01 |
| Q5H9L2 | Transcription elongation factor A | 1.76 | 0.01 |
| | protein-like 5 | | |
| E9PL10 | Transcription factor BTF3 homolog 4 | 1.33 | <0.01 |
| M0R3C0 | Transcription intermediary factor 1- | 0.78 | 0.04 |
| | beta | | |
| Q00577 | Transcriptional activator protein | 1.06 | 0.04 |
| | Pur-alpha | | |
| B4DQI6 | Transformer-2 protein homolog alpha | 0.77 | 0.02 |
| H7BXF3 | Transformer-2 protein homolog beta | 0.72 | 0.04 |
| J3KQ45 | Trans-Golgi network integral membrane | 0.80 | 0.05 |
| | protein 2 | | |
| F8W888 | Transketolase | 1.44 | <0.01 |
| Q9BSH4 | Translational activator of cytochrome | 1.13 | 0.01 |
| | c oxidase 1 (Coiled-coil domain- | | |
| | containing protein 44) (Translational | | |
| | activator of mitochondrially-encoded | | |
| | cytochrome c oxidase I) | | |
| Q53FP2 | Transmembrane protein 35 | 0.87 | 0.05 |
| Q9BTV4 | Transmembrane protein 43 | −0.80 | 0.05 |
| C9JE81 | Trifunctional enzyme subunit beta, | 0.68 | 0.02 |
| | mitochondrial | | |
| P60174 | Triosephosphate isomerase | 1.00 | 0.05 |
| F2Z2W7 | tRNA (uracil-5-)-methyltransferase | 0.89 | <0.01 |
| | homolog A | | |
| Q9Y3I0 | tRNA-splicing ligase RtcB homolog | 1.15 | 0.01 |
| P28289 | Tropomodulin-1 | 1.00 | 0.04 |
| Q15714 | TSC22 domain family protein 1 | 0.89 | <0.01 |
| F8VXZ7 | Tubulin alpha-1A chain | 0.95 | 0.01 |
| M0QZL7 | Tubulin beta-4A chain | 0.75 | 0.01 |
| D6RG15 | Twinfilin-2 | −0.99 | 0.01 |
| P06241 | Tyrosine-protein kinase Fyn (EC | 0.79 | 0.02 |
| | 2.7.10.2) (Proto-oncogene Syn) | | |
| | (Proto-oncogene c-Fyn) (Src-like | | |
| | kinase) (SLK) (p59-Fyn) | | |
| E9PLZ4 | Tyrosine-protein phosphatase non- | 0.98 | 0.02 |
| | receptor type 5 | | |
| M0QYR1 | U1 small nuclear ribonucleoprotein 70 | 0.75 | <0.01 |
| | kDa (Fragment) | | |
| Q9UHD9 | Ubiquilin-2 | 1.15 | 0.01 |
| K7EJ02 | UBX domain-containing protein 6 | −0.78 | 0.01 |
| Q14376 | UDP-glucose 4-epimerase | 0.82 | 0.02 |
| E9PPU6 | Uncharacterized protein | −0.70 | 0.01 |
| Q9BXV9 | Uncharacterized protein C14orf142 | 0.70 | 0.03 |
| H0YEV9 | Unconventional myosin-XVIIIa | −0.81 | 0.02 |
| Q9H3H3 | UPF0696 protein C11orf68 | 0.83 | 0.01 |
| K7ELW1 | UV excision repair protein RAD23 | 0.81 | 0.01 |
| | homolog A | | |
| Q5W0S4 | UV excision repair protein RAD23 | 0.96 | <0.01 |
| | homolog B | | |
| Q709C8 | Vacuolar protein sorting-associated | 1.45 | 0.03 |
| | protein 13C | | |
| Q6EMK4 | Vasorin | 0.80 | 0.04 |
| P49748 | Very long-chain specific acyl-CoA | 0.91 | 0.01 |
| | dehydrogenase, mitochondrial | | |
| B0YJC4 | Vimentin | 0.80 | 0.03 |
| H0Y715 | Voltage-dependent calcium channel | 0.87 | 0.01 |
| | subunit alpha-2/delta-1 | | |
| B5MCX6 | V-set and transmembrane domain- | 0.81 | 0.01 |
| | containing protein 2A | | |
| K7ERA0 | V-type proton ATPase subunit a | 0.74 | <0.01 |
| Q8N8Y2 | V-type proton ATPase subunit d 2 | 1.66 | 0.01 |
| Q15904 | V-type proton ATPase subunit S1 | 1.00 | 0.02 |
| Q8TF74 | WAS/WASL-interacting protein family | 1.71 | 0.02 |
| | member 2 | | |
| H0YMF9 | WD repeat-containing protein 61 | −2.20 | <0.01 |
| Q9UPY6 | Wiskott-Aldrich syndrome protein | 0.77 | 0.03 |
| | family member 3 | | |
| P61129 | Zinc finger CCCH domain-containing | 1.08 | 0.04 |
| | protein 6 | | |
| Q9ULF5 | Zinc transporter ZIP10 | 0.76 | 0.02 |

In one embodiment, the panel of biomarkers comprises at least Basigin.

In another embodiment, the panel of biomarkers comprises at least Cytochrome c oxidase subunit 7A-related protein, mitochondrial.

In another embodiment, the panel of biomarkers comprises at least Basigin and Cytochrome c oxidase subunit 7A-related protein, mitochondrial.

Basigin or BSG or extracellular matrix metalloproteinase inducer (EMMPRIN) or cluster of differentiation 147 (CD147) is a type I integral membrane receptor that has many ligands, including the cyclophilin (CyP) proteins Cyp-A and CyP-B and certain integrins. Basigin has metalloproteinase-inducing ability and it also regulates several distinct functions, such as spermatogenesis, expression of the monocarboxylate transporter and the responsiveness of lymphocytes. It is expressed by many cell types, including epithelial cells, endothelial cells and leukocytes. The human Basigin protein contains 269 amino acids that form two heavily glycosylated C2 type immunoglobulin-like domains at the N-terminal extracellular portion. A second form of Basigin has also been characterized that contains one additional immunoglobulin-like domain in its extracellular portion. Its amino acid sequence is depicted in SEQ ID NO: 30.

Cytochrome c oxidase subunit 7A-related protein, mitochondrial or Cox7r is an enzyme encoded by the COX7A2L gene in humans. Cytochrome c oxidase subunit 7A-related protein, mitochondrial is a component of the Cytochrome c oxidase (COX), which is the terminal component of the mitochondrial respiratory chain and catalyzes the electron transfer from reduced cytochrome c to oxygen. Its amino acid sequence is depicted in SEQ ID NO: 31.

The embodiments relating to the biomarkers selected from Table 5, as described above, are equally applicable to all other embodiments of the third aspect of the invention and to all other aspects of the invention where biomarkers selected from Table 5 are involved.

In one embodiment, the panel of biomarker comprises at least one biomarker selected from Table 5 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one biomarker selected from Groups A, B, C or D. More preferably the biomarker selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

The biomarkers listed in Table 5 may belong to specific pathways which are known to be relevant in the pathology of AD. They may be further grouped in specific lists as those illustrated below in Tables 6 to 13 according to specific GO terms (http://geneontology.org/).

One GO term used to analyse the data was the term “Synap*”. Biomarkers of Table 5 which are registered as part of this specific pathway are shown in Table 6.

In one embodiment, the panel of biomarkers according to the invention comprises one or more, preferably two or more biomarkers selected from Table 6.

TABLE 6
Proteins with GO Term ‘Synap*’ found
to be regulated in CSF of AD patients

| | | Log2 | |
|---|---|---|---|
| UniProtKB | | AD/ | |
| Accession | | Con- | P |
| Number | Protein name | trol | value |
| P05067 | Amyloid beta A4 protein | 0.90 | <0.01 |
| Q9UQM7 | Calcium/calmodulin-dependent protein | 0.81 | 0.02 |
| | kinase type II subunit alpha | | |
| Q16566 | Calcium/calmodulin-dependent protein | 0.85 | 0.03 |
| | kinase type IV | | |
| C9IZ88 | Catenin alpha-2 | 2.11 | 0.01 |
| Q5JYX0 | Cell division control protein 42 homolog | 1.04 | 0.05 |
| I3L0M4 | Coiled-coil domain-containing protein 43 | 0.99 | 0.01 |
| Q8NFT8 | Delta and Notch-like epidermal growth | 1.02 | 0.01 |
| | factor-related receptor | | |
| P50570 | Dynamin-2 | 0.89 | 0.03 |
| P42261 | Glutamate receptor 1 | 1.13 | 0.04 |
| P01112 | GTPase HRas | 1.09 | 0.01 |
| M0R2G0 | Leucine-rich repeat-containing protein 4B | 0.76 | <0.01 |
| Q9Y4C0 | Neurexin-3 | 0.93 | <0.01 |
| Q9HDB5 | Neurexin-3-beta | 0.98 | <0.01 |
| P61601 | Neurocalcin-delta | 1.33 | 0.03 |
| Q9UBB6 | Neurochondrin | 0.80 | 0.02 |
| Q8NFZ4 | Neuroligin-2 | 0.96 | 0.04 |
| P47972 | Neuronal pentraxin-2 | 1.02 | 0.01 |
| Q96HC4 | PDZ and LIM domain protein 5 | 0.75 | 0.02 |
| I3L495 | Platelet-activating factor | 0.97 | 0.02 |
| | acetylhydrolase IB subunit alpha | | |
| Q09470 | Potassium voltage-gated channel subfamily | 0.73 | 0.04 |
| | A member 1 | | |
| P01303 | Pro-neuropeptide Y | 0.86 | 0.04 |
| Q02156 | Protein kinase C epsilon type | 0.99 | 0.02 |
| Q9Y6V0 | Protein piccolo | 0.93 | 0.04 |
| Q9Y4G8 | Rap guanine nucleotide exchange factor 2 | 0.72 | 0.01 |
| P61278 | Somatostatin | 1.03 | 0.03 |
| P17600 | Synapsin-1 | 0.95 | 0.03 |
| Q92777 | Synapsin-2 | 0.86 | 0.03 |
| H7C4W3 | Synaptophysin | 2.38 | 0.03 |

In one embodiment, the panel of biomarker comprises at least one biomarker selected from Table 6 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one biomarker selected from Groups A, B, C or D. More preferably the biomarker selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

Another GO term used to analyse the data was the term “Phosphoryl*”. Biomarkers of Table 5 which are registered as part of this specific pathway are shown in Table 7.

In another embodiment, the panel of biomarkers according to the invention comprises one or more, preferably two or more biomarkers selected from Table 7.

TABLE 7
Proteins with GO Term ‘Phosporyl*’ found
to be regulated in CSF of AD patients

| | | Log2 | |
|---|---|---|---|
| UniProtKB | | AD/ | |
| Accession | | Con- | P |
| Number | Protein name | trol | value |
| Q8IZP0 | Abl interactor 1 | 1.04 | 0.01 |
| P05067 | Amyloid beta A4 protein | 0.90 | <0.01 |
| P03950 | Angiogenin | −0.76 | 0.03 |
| P17858 | ATP-dependent 6-phosphofructokinase, | 0.68 | 0.02 |
| | liver type | | |
| P11274 | Breakpoint cluster region protein | 0.99 | 0.01 |
| Q9UQM7 | Calcium/calmodulin-dependent protein | 0.81 | 0.02 |
| | kinase type II subunit alpha | | |
| Q16566 | Calcium/calmodulin-dependent protein | 0.85 | 0.03 |
| | kinase type IV | | |
| P31321 | cAMP-dependent protein kinase type I- | 1.56 | 0.04 |
| | beta regulatory subunit | | |
| Q5JYX0 | Cell division control protein 42 | 1.04 | 0.05 |
| | homolog | | |
| O75367 | Core histone macro-H2A.1 | 0.67 | 0.01 |
| P14927 | Cytochrome b-c1 complex subunit 7 | 0.98 | 0.05 |
| Q08495 | Dematin | 0.76 | 0.01 |
| P20827 | Ephrin-A1 | 0.86 | 0.05 |
| E5RGR6 | GDNF family receptor alpha-2 | 1.06 | <0.01 |
| P01112 | GTPase HRas | 1.09 | 0.01 |
| H0YB38 | Inositol monophosphatase 3 | 1.12 | 0.04 |
| K7EKJ9 | Interleukin enhancer-binding factor 3 | 1.57 | 0.04 |
| A2A2V1 | Major prion protein | 1.20 | <0.01 |
| Q15746 | Myosin light chain kinase, smooth | 1.08 | 0.01 |
| | muscle | | |
| O14936 | Peripheral plasma membrane protein | 1.07 | 0.04 |
| | CASK | | |
| A8MTF1 | Phosphatidylinositol 4-kinase alpha | 0.71 | 0.01 |
| A6NDG6 | Phosphoglycolate phosphatase | 1.05 | 0.03 |
| Q9H008 | Phospholysine phosphohistidine | 0.74 | 0.03 |
| | inorganic pyrophosphate phosphatase | | |
| Q02156 | Protein kinase C epsilon type | 0.99 | 0.02 |
| Q5SYT8 | Protein NAMPTL | 0.83 | 0.03 |
| O75323 | Protein NipSnap homolog 2 | 1.61 | 0.02 |
| Q96A00 | Protein phosphatase 1 regulatory | 0.70 | 0.05 |
| | subunit 14A | | |
| O43353 | Receptor-interacting serine/threonine- | 0.78 | 0.04 |
| | protein kinase 2 | | |
| P23471 | Receptor-type tyrosine-protein | 0.90 | <0.01 |
| | phosphatase zeta | | |
| O75326 | Semaphorin-7A | 0.85 | <0.01 |
| M0R3C0 | Transcription intermediary factor 1- | 0.78 | 0.04 |
| | beta | | |
| P06241 | Tyrosine-protein kinase Fyn (EC | 0.79 | 0.02 |
| | 2.7.10.2) (Proto-oncogene Syn) (Proto- | | |
| | oncogene c-Fyn) (Src-like kinase) | | |
| | (SLK) (p59-Fyn) | | |

In one embodiment, the panel of biomarker comprises at least one biomarker selected from Table 7 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one biomarker selected from Groups A, B, C or D. More preferably the biomarker selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

Another GO term used to analyse the data was the term “Stress”. Biomarkers of Table 5 which are registered as part of this specific pathway are shown in Table 8.

In another embodiment, the panel of biomarkers according to the invention comprises one or more, preferably two or more biomarkers selected from Table 8.

TABLE 8
Proteins with GO Term ‘Stress’ found
to be regulated in CSF of AD patients

| | | Log2 | |
|---|---|---|---|
| UniProtKB | | AD/ | |
| Accession | | Con- | P |
| Number | Protein name | trol | value |
| P11766 | Alcohol dehydrogenase class-3 | 0.87 | 0.05 |
| P05067 | Amyloid beta A4 protein | 0.90 | <0.01 |
| E9PMA0 | Apoptosis-inducing factor 1, | 0.69 | 0.03 |
| | mitochondrial | | |
| P29972 | Aquaporin-1 | 0.90 | <0.01 |
| Q99674 | Cell growth regulator with EF hand | 0.86 | <0.01 |
| | domain protein 1 | | |
| H0YIV0 | Endoplasmin | 1.18 | 0.04 |
| P07203 | Glutathione peroxidase 1 | 1.13 | 0.02 |
| C9J3N8 | Heat shock protein beta-1 | −0.91 | 0.01 |
| Q9Y4L1 | Hypoxia up-regulated protein 1 | 0.86 | 0.02 |
| Q13136 | Liprin-alpha-1 | 0.85 | 0.01 |
| A2A2V1 | Major prion protein | 1.20 | <0.01 |
| E5RJK1 | Mitochondrial peptide methionine | 0.87 | <0.01 |
| | sulfoxide reductase | | |
| P32119 | Peroxiredoxin-2 | 0.96 | 0.03 |
| P60903 | Protein S100-A10 | 0.75 | 0.02 |
| O43353 | Receptor-interacting serine/threonine- | 0.78 | 0.04 |
| | protein kinase 2 | | |
| Q99250 | Sodium channel protein type 2 subunit | 0.80 | 0.05 |
| | alpha | | |
| Q9BX66 | Sorbin and SH3 domain-containing | 0.82 | 0.05 |
| | protein 1 | | |

In yet another embodiment, the panel of biomarker comprises at least one biomarker selected from Table 8 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one or more biomarker selected from Groups A, B, C or D. More preferably the one or more biomarkers selected from Groups A, B, C or D is:
i) protein phosphatase 1 regulatory subunit 14A comprising or having the amino acid sequence of SEQ ID NO:1 or an isoform or a variant or a fragment thereof;
or
ii) 2,3′-cyclic-nucleotide 3′-phosphodiesterase comprising or having the amino acid sequence of SEQ ID NO:2 or an isoform or a variant or a fragment thereof.

Another GO term used to analyse the data was the term “Calcium”. Biomarkers of Table 5 which are registered as part of this specific pathway are shown in Table 9.

In another embodiment, the panel of biomarkers according to the invention comprises one or more, preferably two or more biomarkers selected from Table 9.

TABLE 9
Proteins with GO Term ‘Calcium’ found
to be regulated in CSF of AD patients

| | | Log2 | |
|---|---|---|---|
| UniProtKB | | AD/ | |
| Accession | | Con- | P |
| Number | Protein name | trol | value |
| P05067 | Amyloid beta A4 protein | 0.90 | <0.01 |
| E7EV01 | Ankyrin repeat and SOCS box protein 2 | 1.68 | 0.01 |
| P07686 | Beta-hexosaminidase subunit beta | 1.49 | <0.01 |
| P19022 | Cadherin-2 | 1.17 | 0.05 |
| Q9UQM7 | Calcium/calmodulin-dependent protein | 0.81 | 0.02 |
| | kinase type II subunit alpha | | |
| P07384 | Calpain-1 catalytic subunit | 1.06 | 0.03 |
| Q96A33 | Coiled-coil domain-containing protein | 0.79 | 0.02 |
| | 47 | | |
| Q08495 | Dematin | 0.76 | 0.01 |
| P78417 | Glutathione S-transferase omega-1 | 1.13 | 0.01 |
| P13645 | Keratin, type I cytoskeletal 10 | 0.66 | 0.02 |
| P01042 | Kininogen-1 | −0.67 | 0.02 |
| Q15746 | Myosin light chain kinase, smooth | 1.08 | 0.01 |
| | muscle | | |
| P61601 | Neurocalcin-delta | 1.33 | 0.03 |
| Q9UBV8 | Peflin | 0.98 | 0.03 |
| O14936 | Peripheral plasma membrane protein | 1.07 | 0.04 |
| | CASK | | |
| P01303 | Pro-neuropeptide Y | 0.86 | 0.04 |
| Q02156 | Protein kinase C epsilon type | 0.99 | 0.02 |
| Q15493 | Regucalcin | 1.91 | 0.03 |
| H7C5W9 | Sarcoplasmic/endoplasmic reticulum | 0.77 | 0.05 |
| | calcium ATPase 2 | | |
| P06241 | Tyrosine-protein kinase Fyn (EC | 0.79 | 0.02 |
| | 2.7.10.2) (Proto-oncogene Syn) (Proto- | | |
| | oncogene c-Fyn) (Src-like kinase) | | |
| | (SLK) (p59-Fyn) | | |

In one another embodiment, the panel of biomarker comprises at least one biomarker selected from Table 9 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one biomarker selected from Groups A, B, C or D. More preferably the biomarker selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

Another GO term used to analyse the data was the term “Cytoskelet*”. Biomarkers of Table 5 which are registered as part of this specific pathway are shown in Table 10.

In another embodiment, the panel of biomarker according to the invention comprises one or more, preferably two or more biomarkers selected from Table 10.

TABLE 10
Proteins with GO Term ‘Cytoskelet*’ found
to be regulated in CSF of AD patients

| | | Log2 | |
|---|---|---|---|
| UniProtKB | | AD/ | |
| Accession | | Con- | P |
| Number | Protein name | trol | value |
| Q8IZP0 | Abl interactor 1 | 1.04 | 0.01 |
| E9PF58 | Actin-related protein 2/3 complex | 0.94 | 0.02 |
| | subunit 1A | | |
| P16157 | Ankyrin-1 | 0.86 | 0.01 |
| P04114 | Apolipoprotein B-100 | 0.70 | 0.04 |
| Q4VXN1 | Band 4.1-like protein 1 | 1.36 | 0.01 |
| Q562R1 | Beta-actin-like protein 2 | 0.77 | 0.05 |
| C9IZ88 | Catenin alpha-2 | 2.11 | 0.01 |
| Q14019 | Coactosin-like protein | 0.68 | 0.02 |
| Q08495 | Dematin | 0.76 | 0.01 |
| P60981 | Destrin | 0.93 | 0.05 |
| Q15334 | Lethal(2) giant larvae protein homolog | 0.74 | 0.04 |
| | 1 | | |
| O14745 | Na(+)/H(+) exchange regulatory | 1.06 | 0.05 |
| | cofactor NHE-RF1 | | |
| Q96HC4 | PDZ and LIM domain protein 5 | 0.75 | 0.02 |
| O14936 | Peripheral plasma membrane protein | 1.07 | 0.04 |
| | CASK | | |
| Q02156 | Protein kinase C epsilon type | 0.99 | 0.02 |
| Q9Y6V0 | Protein piccolo | 0.93 | 0.04 |
| O43353 | Receptor-interacting serine/threonine- | 0.78 | 0.04 |
| | protein kinase 2 | | |
| Q5TZA2 | Rootletin | 0.77 | 0.03 |
| B4DLV4 | Serine hydroxymethyltransferase | −1.13 | 0.03 |
| P34897 | Serine hydroxymethyltransferase, | −1.81 | 0.04 |
| | mitochondrial | | |
| P28289 | Tropomodulin-1 | 1.00 | 0.04 |
| B0YJC4 | Vimentin | 0.80 | 0.03 |
| Q8TF74 | WAS/WASL-interacting protein family | 1.71 | 0.02 |
| | member 2 | | |
| Q9UPY6 | Wiskott-Aldrich syndrome protein | 0.77 | 0.03 |
| | family member 3 | | |

In another embodiment, the panel of biomarker comprises at least one biomarker selected from Table 10 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one or more biomarkers selected from Groups A, B, C or D. More preferably the one or more biomarkers selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

Yet another GO term used to analyse the data was the term “Mitochondri*”. Biomarkers of Table 5 which are registered as part of this specific pathway are shown in Table 11.

In another embodiment, the panel of biomarkers according to the invention comprises one or more, preferably two or more biomarkers selected from Table 11.

TABLE 11
Proteins with GO Term ‘Mitochondri*’ found
to be regulated in CSF of AD patients

| | | Log2 | |
|---|---|---|---|
| UniProtKB | | AD/ | |
| Accession | | Con- | P |
| Number | Protein name | trol | value |
| K7EJ68 | 3-ketoacyl-CoA thiolase, mitochondrial | 0.91 | 0.01 |
| P25325 | 3-mercaptopyruvate sulfurtransferase | 0.81 | 0.01 |
| O14561 | Acyl carrier protein, mitochondrial | 0.72 | 0.02 |
| P07108 | Acyl-CoA-binding protein | 1.18 | 0.02 |
| O14734 | Acyl-coenzyme A thioesterase 8 | 0.76 | <0.01 |
| P12235 | ADP/ATP translocase 1 | 0.71 | 0.03 |
| P11766 | Alcohol dehydrogenase class-3 | 0.87 | 0.05 |
| E9PMA0 | Apoptosis-inducing factor 1, | 0.69 | 0.03 |
| | mitochondrial | | |
| O43776 | Asparagine-tRNA ligase, cytoplasmic | 1.37 | 0.03 |
| K7EJP1 | ATP synthase subunit alpha, | 0.77 | 0.04 |
| | mitochondrial | | |
| Q86VU5 | Catechol O-methyltransferase domain- | 1.06 | 0.01 |
| | containing protein 1 | | |
| Q8NI60 | Chaperone activity of bc1 complex-like, | 0.78 | 0.01 |
| | mitochondrial | | |
| H3BP04 | Cytochrome b-c1 complex subunit 2, | 0.67 | 0.02 |
| | mitochondrial | | |
| P14927 | Cytochrome b-c1 complex subunit 7 | 0.98 | 0.05 |
| P47985 | Cytochrome b-c1 complex subunit Rieske, | 1.13 | 0.04 |
| | mitochondrial | | |
| Q5JTJ3 | Cytochrome c oxidase assembly factor 6 | 1.23 | 0.01 |
| | homolog | | |
| K7EQD3 | Cytochrome c oxidase subunit 6B1 | 0.88 | 0.02 |
| E5RJZ1 | Cytochrome c oxidase subunit 7A-related | 4.19 | 0.05 |
| | protein, mitochondrial | | |
| Q9NVH1 | DnaJ homolog subfamily C member 11 | 0.94 | 0.01 |
| Q92506 | Estradiol 17-beta-dehydrogenase 8 | 0.87 | 0.04 |
| Q92731 | Estrogen receptor beta | 1.73 | 0.01 |
| G3V1D1 | Ferritin | 1.37 | 0.03 |
| P07203 | Glutathione peroxidase 1 | 1.13 | 0.02 |
| P23434 | Glycine cleavage system H protein, | 0.94 | 0.01 |
| | mitochondrial | | |
| P08107 | Heat shock 70 kDa protein 1A/1B | 0.70 | 0.03 |
| F5GWX2 | Heme-binding protein 1 | 1.24 | <0.01 |
| B1AR61 | Hexokinase-1 | 0.83 | 0.02 |
| Q16775 | Hydroxyacylglutathione hydrolase, | 0.85 | 0.04 |
| | mitochondrial | | |
| K7EKJ9 | Interleukin enhancer-binding factor 3 | 1.57 | 0.04 |
| Q5VVL7 | Lipoamide acyltransferase component of | 0.84 | 0.03 |
| | branched-chain alpha-keto acid | | |
| | dehydrogenase complex, mitochondrial | | |
| A8MW50 | L-lactate dehydrogenase | 0.72 | 0.02 |
| F5GZQ4 | L-lactate dehydrogenase A chain | 0.68 | <0.01 |
| O95573 | Long-chain-fatty-acid-CoA ligase 3 | 0.82 | 0.01 |
| C9JF79 | Malate dehydrogenase | 1.07 | 0.04 |
| Q5HYI7 | Metaxin-3 | 1.07 | <0.01 |
| Q13825 | Methylglutaconyl-CoA hydratase, | 1.50 | 0.03 |
| | mitochondrial | | |
| Q9H936 | Mitochondrial glutamate carrier 1 | 0.66 | <0.01 |
| G3V502 | Mitochondrial import inner membrane | 0.92 | 0.03 |
| | translocase subunit Tim9 | | |
| E5RJK1 | Mitochondrial peptide methionine | 0.87 | <0.01 |
| | sulfoxide reductase | | |
| Q10713 | Mitochondrial-processing peptidase | 0.70 | 0.04 |
| | subunit alpha | | |
| D6RAU3 | Mitogen-activated protein kinase 10 | 0.65 | 0.04 |
| O95865 | N(G),N(G)-dimethylarginine | 0.83 | 0.01 |
| | dimethylaminohydrolase 2 | | |
| Q4G0N4 | NAD kinase 2, mitochondrial | 0.96 | <0.01 |
| D6RAI5 | NAD(P) transhydrogenase, mitochondrial | 0.86 | <0.01 |
| F8VRD8 | NADH dehydrogenase [ubiquinone] 1 alpha | 1.00 | 0.05 |
| | subcomplex subunit 12 | | |
| O95182 | NADH dehydrogenase [ubiquinone] 1 alpha | 0.73 | 0.02 |
| | subcomplex subunit 7 | | |
| Q16795 | NADH dehydrogenase [ubiquinone] 1 alpha | 0.74 | 0.04 |
| | subcomplex subunit 9, mitochondrial | | |
| E9PQ68 | NADH dehydrogenase [ubiquinone] 1 beta | −0.85 | <0.01 |
| | subcomplex subunit 8, mitochondrial | | |
| P56181 | NADH dehydrogenase [ubiquinone] | 0.70 | 0.03 |
| | flavoprotein 3, mitochondrial | | |
| Q9UHQ9 | NADH-cytochrome b5 reductase 1 | 0.72 | 0.02 |
| Q02790 | Peptidyl-prolyl cis-trans isomerase | 0.73 | 0.04 |
| | FKBP4 | | |
| F8VVM2 | Phosphate carrier protein, mitochondrial | 0.78 | 0.01 |
| P36969 | Phospholipid hydroperoxide glutathione | 0.75 | 0.02 |
| | peroxidase, mitochondrial | | |
| Q9UHV9 | Prefoldin subunit 2 | 0.72 | 0.04 |
| Q9H7Z7 | Prostaglandin E synthase 2 | 0.98 | 0.02 |
| Q02156 | Protein kinase C epsilon type | 0.99 | 0.02 |
| Q9BPW8 | Protein NipSnap homolog 1 | 0.80 | 0.04 |
| O75323 | Protein NipSnap homolog 2 | 1.61 | 0.02 |
| K7EIR2 | Protein QIL1 | −0.74 | 0.03 |
| H3BTN5 | Pyruvate kinase | 0.71 | 0.03 |
| H0YLG5 | Regulator of microtubule dynamics | 0.93 | 0.04 |
| | protein 3 | | |
| B4DLV4 | Serine hydroxymethyltransferase | −1.13 | 0.03 |
| P34897 | Serine hydroxymethyltransferase, | −1.81 | 0.04 |
| | mitochondrial | | |
| P45954 | Short/branched chain specific acyl-CoA | 1.11 | <0.01 |
| | dehydrogenase, mitochondrial | | |
| C9K0U8 | Single-stranded DNA-binding protein, | 0.67 | 0.04 |
| | mitochondrial | | |
| P31040 | Succinate dehydrogenase [ubiquinone] | 1.05 | 0.03 |
| | flavoprotein subunit, mitochondrial | | |
| C9J8Q5 | Succinate-semialdehyde dehydrogenase, | 0.77 | 0.01 |
| | mitochondrial | | |
| P10599 | Thioredoxin | 0.93 | 0.03 |
| Q9BSH4 | Translational activator of cytochrome c | 1.13 | 0.01 |
| | oxidase 1 (Coiled-coil domain-containing | | |
| | protein 44) (Translational activator of | | |
| | mitochondrially-encoded cytochrome c | | |
| | oxidase I) | | |
| C9JE81 | Trifunctional enzyme subunit beta, | 0.68 | 0.02 |
| | mitochondrial | | |
| P06241 | Tyrosine-protein kinase Fyn (EC | 0.79 | 0.02 |
| | 2.7.10.2) (Proto-oncogene Syn) (Proto- | | |
| | oncogene c-Fyn) (Src-like kinase) (SLK) | | |
| | (p59-Fyn) | | |
| Q6EMK4 | Vasorin | 0.80 | 0.04 |
| P49748 | Very long-chain specific acyl-CoA | 0.91 | 0.01 |
| | dehydrogenase, mitochondrial | | |

In another embodiment, the panel of biomarker comprises at least one biomarker selected from Table 11 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one or more biomarkers selected from Groups A, B, C or D. More preferably the one or more biomarkers selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

The biomarkers of Table 5 were also analysed with the GO terms “Vesicle” and “Insuline”. Those which are registered as part of this specific pathways are shown in Table 12 and Table 13, respectively.

In another embodiment, the panel of biomarker according to the invention comprises one or more, preferably two or more biomarkers selected from Table 12 or Table 13.

TABLE 12
Proteins with GO Term ‘Vesicle’ found
to be regulated in CSF of AD patients

| | | Log2 | |
|---|---|---|---|
| UniProtKB | | AD/ | |
| Accession | | Con- | P |
| Number | Protein name | trol | value |
| P16157 | Ankyrin-1 | 0.86 | 0.01 |
| B0YIW6 | Archain 1 | −1.26 | 0.01 |
| Q9UPT6 | C-Jun-amino-terminal kinase- | −0.85 | 0.01 |
| | interacting protein 3 | | |
| D6RJD1 | Clathrin light chain B | 0.84 | 0.02 |
| P53618 | Coatomer subunit beta | 1.07 | 0.03 |
| H0Y8X7 | Coatomer subunit gamma-1 | 0.69 | 0.03 |
| P50570 | Dynamin-2 | 0.89 | 0.03 |
| P61601 | Neurocalcin-delta | 1.33 | 0.03 |
| I3L495 | Platelet-activating factor | 0.97 | 0.02 |
| | acetylhydrolase IB subunit alpha | | |
| Q9Y6V0 | Protein piccolo | 0.93 | 0.04 |
| Q8WUD1 | Ras-related protein Rab-2B | 0.80 | 0.01 |
| Q99523 | Sortilin | 0.68 | 0.03 |
| P17600 | Synapsin-1 | 0.95 | 0.03 |

TABLE 13
Proteins with GO Term ‘Insulin’ found
to be regulated in CSF of AD patients

| | | Log2 | |
|---|---|---|---|
| UniProtKB | | AD/ | |
| Accession | | Con- | P |
| Number | Protein name | trol | value |
| P12235 | ADP/ATP translocase 1 | 0.71 | 0.03 |
| P17174 | Aspartate aminotransferase, | 0.99 | 0.03 |
| | cytoplasmic | | |
| P17858 | ATP-dependent 6-phosphofructokinase, | 0.68 | 0.02 |
| | liver type | | |
| P31321 | cAMP-dependent protein kinase type I- | 1.56 | 0.04 |
| | beta regulatory subunit | | |
| Q9UKG1 | DCC-interacting protein 13-alpha | 0.79 | 0.01 |
| P01112 | GTPase HRas | 1.09 | 0.01 |
| P63092 | Guanine nucleotide-binding protein | 1.00 | 0.03 |
| | G(s) subunit alpha isoforms short | | |
| P16519 | Neuroendocrine convertase 2 | 1.52 | 0.00 |
| O15240 | Neurosecretory protein VGF | 1.12 | 0.00 |
| Q9BZF1 | Oxysterol-binding protein-related | 0.78 | 0.04 |
| | protein 8 | | |
| Q02156 | Protein kinase C epsilon type | 0.99 | 0.02 |
| Q9Y6V0 | Protein piccolo | 0.93 | 0.04 |
| Q9BX66 | Sorbin and SH3 domain-containing | 0.82 | 0.05 |
| | protein 1 | | |
| Q99523 | Sortilin | 0.68 | 0.03 |
| Q8N8Y2 | V-type proton ATPase subunit d 2 | 1.66 | 0.01 |

In other embodiments, the panel of biomarker comprises at least one biomarker selected from Table 12 or Table 13 and at least one biomarker selected from tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid.

Preferably, the panel also comprises one or more biomarkers selected from Groups A, B, C or D. More preferably the one or more biomarkers selected from Groups A, B, C or D is:
i) protein phosphatase 1 regulatory subunit 14A comprising or having the amino acid sequence of SEQ ID NO:1 or an isoform or a variant or a fragment thereof;
or
ii) 2′,3′-cyclic-nucleotide 3′-phosphodiesterase comprising or having the amino acid sequence of SEQ ID NO:2 or an isoform or a variant or a fragment thereof.

All non-modified peptides for each protein were summed for three control and three AD cases. The log 2 ratio and p-value of each protein was then calculated. Proteins with 2 or more peptides, >60% regulation and p<0.05 were selected as biomarkers of synaptic dysfunction (Table 6), dysregulated phosphorylation (Table 7), oxidative stress (Table 8), dysregulated calcium signalling (Table 9), dysregulated cytoskeleton (Table 10), mitochondrial damage (Table 11), abnormal vesicle function (Table 12) and dysfunctional insulin signalling (Table 13).

The panels of biomarkers described herein are useful for diagnosing, for staging, for assessing the likelihood of developing a neurocognitive disorder, and for assessing the response to a drug for treating a neurocognitive disorder, such as a neurocognitive disease characterized by tau toxicity, for example a tauopathy and in particular Alzheimer's disease. The use of these panels of biomarkers according to the present invention in any of such methods has considerable advantages.

Firstly, the biomarkers according to the present invention represent the translation of tau toxicity and resulting changes in pathways that occur in the brain into a peripheral signal in a peripheral tissue such as CSF and blood. Hence, they allow replacing tissue testing with a peripheral fluid testing. This represents a great advantage especially as the tissue primarily affected in neurocognitive disorder is the brain tissue. Brain biopsies are not carried out unless post-mortem.

Secondly, the biomarkers according to the present invention have been selected as those being capable to translate the specific stage of a neurocognitive disorder characterized by tau toxicity, such as Alzheimer's disease. This also represents a great advantage as currently clinicians assess the advancement of a neurocognitive diseases like AD through a battery of psychometric tests, which, albeit being somewhat indicative of the disease progression, may not be precise on the stage of the disease, thus, making it particularly difficult to select those therapies which have been developed and approved for a specific stage.

Thirdly, these biomarkers further comprise proteins which are not those typically reported in the literature or currently used in the clinical setting as biomarkers for tauopathies, thus providing clinicians with additional tools for identifying and distinguishing, even at an early stage, subjects who have a neurocognitive disorder characterized by tau toxicity, such as AD and subjects who, despite presenting symptoms of neurocognitive impairment are not affected by the early signs of AD.

Hence, the present invention provides for a method for diagnosing a neurocognitive disorder in a subject, the method comprising:
a) assaying a sample obtained from said subject for biomarkers of a panel as defined herein;
b) measuring in said sample a concentration or an amount of each of the biomarkers of said panel;
c) determining whether that subject has a neurocognitive disorder by comparing said concentration or amount of each of the biomarkers of the panel in said sample with reference concentrations or amounts of said biomarkers;
   wherein the panel of biomarkers is selected from a panel comprising: I)
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

Preferably, the neurocognitive disorder is characterized by tau-toxicity; more preferably the neurocognitive disorder is a tauopathy selected from the group of Alzheimer's disease, frontotemporal dementia with Parkinsonism linked to chromosome 17 (FTDP-17), progressive supranuclear palsy (PSP), Pick's disease, corticobasal degeneration, multisystem atrophy (MSA), neurobasal degeneration with iron accumulation, type 1 (Hallervorden-Spatz), argyrophilic grain dementia, Down's syndrome, diffuse neurofibrillary tangles with calcification, dementia pugilistica, Gerstmann-Straussler-Scheinker disease, myotonic dystrophy, Niemann-Pick disease type C, progressive subcortical gliosis, prion protein cerebral amyloid angiopathy, tangle only dementia, postencephalitic parkinsonism, subacute sclerosing panencephalitis, Creutzfeldt-Jakob disease, amyotrophic lateral sclerosis/parkinsonism-dementia complex, non-Guamanian motor neuron disease with neurofibrillary tangles/dementia, chronic traumatic encephalopathy, alpha-synucleinopathies, Parkinson's disease or combinations thereof.

Even more preferably the tauopathy is Alzheimer's disease.

The present invention also provide for a method for staging a neurocognitive disorder in a subject, the method comprising:
a) assaying a sample obtained from said subject for biomarkers of a panel as defined herein;
b) measuring in said sample a concentration or an amount of each of the biomarkers of said panel;
c) determining the stage of the neurocognitive disorder in said subject by comparing said concentration or amount of each of the biomarkers of the panel in said sample with reference concentrations or amounts of said biomarkers;
   wherein the panel of biomarkers is selected from a panel comprising: I)
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

Preferably, in the staging method according to the invention, the levels of protein phosphatase 1 regulatory subunit 14A are increased in said sample of a subject with an advanced stage of the neurocognitive disorder; and/or the levels of 2′,3′-cyclic-nucleotide 3′-phosphodiesterase are increased in said sample of a subject with an advanced stage of the neurocognitive disorder. In one embodiment, the staging of the neurocognitive disorder is higher (i.e. more advanced stage) the higher the tau expression and hyperphosphorylation. Hence, the stage is tau-dependent.

Preferably, the neurocognitive disorder is characterized by tau-toxicity and more preferably the neurocognitive disorder is a tauopathy selected from the group of Alzheimer's disease, frontotemporal dementia with Parkinsonism linked to chromosome 17 (FTDP-17), progressive supranuclear palsy (PSP), Pick's disease, corticobasal degeneration, multisystem atrophy (MSA), neurobasal degeneration with iron accumulation, type 1 (Hallervorden-Spatz), argyrophilic grain dementia, Down's syndrome, diffuse neurofibrillary tangles with calcification, dementia pugilistica, Gerstmann-Straussler-Scheinker disease, myotonic dystrophy, Niemann-Pick disease type C, progressive subcortical gliosis, prion protein cerebral amyloid angiopathy, tangle only dementia, postencephalitic parkinsonism, subacute sclerosing panencephalitis, Creutzfeldt-Jakob disease, amyotrophic lateral sclerosis/parkinsonism-dementia complex, non-Guamanian motor neuron disease with neurofibrillary tangles/dementia, chronic traumatic encephalopathy, alpha-synucleinopathies, Parkinson's disease or combinations thereof.

Even more preferably the tauopathy is Alzheimer's disease (AD) and the staging is any one of the stages of Braak staging of AD.

Braak staging for AD was firstly described in 1991 (Braak, H. et al. (1991) Acta Neuropathologica 82 (4): 239-59) and comprises:
stages I and II: used when neurofibrillary tangle involvement is confined mainly to the transentorhinal region of the brain;
stages III and IV when there is also involvement of limbic regions such as the hippocampus, and
stages V and VI when there is extensive neocortical involvement

In one preferred embodiment, when the the neurocognitive disorder is AD, the concentration or amount of protein phosphatase 1 regulatory subunit 14A comprising or having the amino acid sequence of SEQ ID NO:1 or an isoform or a variant or a fragment thereof is increased in the sample of AD patients with AD Braak stage V or Braak stage VI with respect to AD patients with AD Braak stage III or stage IV.

In another preferred embodiment, when the the neurocognitive disorder is AD, the concentration or amount of 2′,3′-cyclic-nucleotide 3′-phosphodiesterase comprising or having the amino acid sequence of SEQ ID NO:2 or an isoform or a variant or a fragment thereof is increased in the sample of AD patients with AD Braak stage V or Braak stage VI with respect to AD patients with AD Braak stage III or stage IV.

The invention also provides a method for assessing in a subject the likelihood of developing a neurocognitive disorder, the method comprising:
a) assaying a sample obtained from said subject for biomarkers of a panel as defined herein;
b) measuring in said sample a concentration or an amount of each of the biomarkers of said panel;
c) determining whether that subject is likely to develop a neurocognitive disorder by comparing said concentration or amount of each of the biomarkers of the biomarker panel in said sample with reference concentrations or amounts of said biomarkers;
   wherein the panel of biomarkers is selected from a panel comprising: I)
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

Preferably, the neurocognitive disorder is characterized by tau-toxicity and more preferably the neurocognitive disorder is a tauopathy selected from the group of Alzheimer's disease, frontotemporal dementia with Parkinsonism linked to chromosome 17 (FTDP-17), progressive supranuclear palsy (PSP), Pick's disease, corticobasal degeneration, multisystem atrophy (MSA), neurobasal degeneration with iron accumulation, type 1 (Hallervorden-Spatz), argyrophilic grain dementia, Down's syndrome, diffuse neurofibrillary tangles with calcification, dementia pugilistica, Gerstmann-Straussler-Scheinker disease, myotonic dystrophy, Niemann-Pick disease type C, progressive subcortical gliosis, prion protein cerebral amyloid angiopathy, tangle only dementia, postencephalitic parkinsonism, subacute sclerosing panencephalitis, Creutzfeldt-Jakob disease, amyotrophic lateral sclerosis/parkinsonism-dementia complex, non-Guamanian motor neuron disease with neurofibrillary tangles/dementia, chronic traumatic encephalopathy, alpha-synucleinopathies, Parkinson's disease or combinations thereof.

Even more preferably the tauopathy is Alzheimer's disease.

The present invention also provides for a method for treating a neurocognitive disorder in a subject, the method comprising:
a) assaying a sample obtained from said subject for biomarkers of a panel as defined herein;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers;
d) administering to said subject a drug for treating the neurocognitive disorder;
   wherein the panel of biomarkers is selected from a panel comprising:
   I) i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

Alternatively, this aspect may be formulated as a drug for use in the treatment of a neurocognitive disorder in a subject, wherein the subject is selected by the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers;
   wherein the panel of biomarkers is selected from a panel comprising:
   I) i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.
   or, alternatively,
   as a use of a drug for the manufacture of a medicament for the treatment of a neurocognitive disorder in a subject, wherein the subject is selected by the method comprising:
a) assaying a sample obtained from said subject for the biomarkers of the panel as defined in any one of the first, second and third aspect of the invention, including the embodiments thereof;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers;
   wherein the panel of biomarkers is selected from a panel comprising:
   I) i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

In one embodiment, when the sample is a brain sample, the concentration or amount of protein phosphatase 1 regulatory subunit 14A comprising or having the amino acid sequence of SEQ ID NO:1 or an isoform or a variant or a fragment thereof; and/or 2′,3′-cyclic-nucleotide 3′-phosphodiesterase comprising or having the amino acid sequence of SEQ ID NO:2 or an isoform or a variant or a fragment thereof will decrease in response to the administration of the drug for treating the neurocognitive disorder.

In another embodiment, when the sample is CSF, the concentration or amount of protein phosphatase 1 regulatory subunit 14A comprising or having the amino acid sequence of SEQ ID NO:1 or an isoform or a variant or a fragment thereof; and/or 2′,3′-cyclic-nucleotide 3′-phosphodiesterase comprising or having the amino acid sequence of SEQ ID NO:2 or an isoform or a variant or a fragment thereof increase or decrease in response to the treatment.

The present invention also provide for a method for assessing the response to a drug for treating a neurocognitive disorder in a subject, wherein the subject has been treated or is being treated with said drug, the method comprises:
a) assaying a sample obtained from said subject for biomarkers of a panel as described herein;
b) measuring in said sample a concentration or an amount of each of the biomarkers of said biomarker panel;
c) determining whether said treatment for Alzheimer's disease is successful by comparing said concentration or amount of each of the biomarkers of the panel in said sample with reference concentrations or amounts of said biomarkers,
   wherein the panel of biomarkers is selected from a panel comprising: I)
i) protein phosphatase 1 regulatory subunit 14A comprising or having the amino acid sequence of SEQ ID NO:1 or an isoform or a variant or a fragment thereof;
and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

Preferably, the neurocognitive disorder in these two aspects of the invention is characterized by tau-toxicity and more preferably the neurocognitive disorder is a tauopathy selected from the group of Alzheimer's disease, frontotemporal dementia with Parkinsonism linked to chromosome 17 (FTDP-17), progressive supranuclear palsy (PSP), Pick's disease, corticobasal degeneration, multisystem atrophy (MSA), neurobasal degeneration with iron accumulation, type 1 (Hallervorden-Spatz), argyrophilic grain dementia, Down's syndrome, diffuse neurofibrillary tangles with calcification, dementia pugilistica, Gerstmann-Straussler-Scheinker disease, myotonic dystrophy, Niemann-Pick disease type C, progressive subcortical gliosis, prion protein cerebral amyloid angiopathy, tangle only dementia, postencephalitic parkinsonism, subacute sclerosing panencephalitis, Creutzfeldt-Jakob disease, amyotrophic lateral sclerosis/parkinsonism-dementia complex, non-Guamanian motor neuron disease with neurofibrillary tangles/dementia, chronic traumatic encephalopathy, alpha-synucleinopathies, Parkinson's disease or combinations thereof.

In one preferred embodiment the tauopathy is Alzheimer's disease.

In one embodiment, the drug for treating the neurocognitive disorder is a kinase inhibitor; preferably the kinase inhibitor is selected from a tau kinase inhibitor or a casein kinase inhibitor, more preferably a casein kinase 1 alpha, beta, gamma, delta or epsilon.

Even more preferably, the kinase inhibitor is a casein kinase 1 delta inhibitor. Casein kinase 1 delta inhibitors are described in WO2012080727 and WO2012080729 which are incorporated herein as reference.

Examples of casein kinase 1 delta inhibitors are 5-(1,3-benzoxazol-2-yl)-4-(pyridin-4-yl)pyrimidin-2-amine (PS110); 2-amino-3-[(thiophen-2-yl)carbonyl]indolizine-1-carboxamide; 2-[3-(pyridin-4-yl)-1H-pyrazol-4-yl]-1,3-benzoxazole; 2-amino-3-[(4-fluorophenyl)carbonyl]indolizine-1-carboxamide (PS278); 2-Methyl-amino-3-[(4-fluorophenyl)carbonyl]indolizine-1-carboxamide 2-amino-3-benzoylindolizine-1-carboxamide; 2-amino-1-[(4-fluorophenyl)carbonyl]-1H-indole-3-carboxamide; combinations thereof; or pharmaceutically acceptable salt or solvate thereof.

The most preferred casein kinase 1 delta inhibitor is selected from 5-(1,3-benzoxazol-2-yl)-4-(pyridin-4-yl)pyrimidin-2-amine (PS110); 2-amino-3-[(4-fluorophenyl)carbonyl]indolizine-1-carboxamide (PS278); 2-Methyl-amino-3-[(4-fluorophenyl)carbonyl]indolizine-1-carboxamide; combinations thereof; or pharmaceutically acceptable salt or solvate thereof.

Preferably, step d) in the method of treatment according to the invention further comprises administering an additional therapeutic agent. In one embodiment, the subject has been treated or is being treated with a kinase inhibitor and the additional therapeutic agent is selected from the group of memantine (e.g. Namenda®), galantamine (e.g. Razadyne®), rivastigmine (e.g. Exelon®), donepezil (e.g. Aricept®), solanezumab, 5HT5 antagonists or combinations thereof. In another embodiment, the subject has been treated or is being treated with an agent selected from the group of memantine (e.g. Namenda®), galantamine (e.g. Razadyne®), rivastigmine (e.g. Exelon®), donepezil (e.g. Aricept®), solanezumab, 5HT5 antagonists or combinations thereof and the additional therapeutic agent is selected from a kinase inhibitor, preferably a casein kinase 1 delta inhibitor, more preferably a casein kinase inhibitor selected from 5-(1,3-benzoxazol-2-yl)-4-(pyridin-4-yl)pyrimidin-2-amine; 2-amino-3-[(4-fluorophenyl)carbonyl]indolizine-1-carboxamide; 2-Methyl-amino-3-[(4-fluorophenyl)carbonyl]indolizine-1-carboxamide; combinations thereof; or pharmaceutically acceptable salt or solvate thereof.

In one embodiment of the invention, when assessing the response to a drug for treating a neurocognitive disorder in a subject, wherein the subject has been treated or is being treated with said drug:
i) said subject has also been treated or is also being treated with an additional therapeutic agent selected from the group of memantine (e.g. Namenda®), galantamine (e.g. Razadyne®), rivastigmine (e.g. Exelon®), donepezil (e.g. Aricept®), solanezumab, 5HT₅ antagonists or combinations thereof; and/or
ii) after step c), the method comprises administering an additional therapeutic agent selected from the group of memantine (e.g. Namenda®), galantamine (e.g. Razadyne®), rivastigmine (e.g. Exelon®), donepezil (e.g. Aricept®), solanezumab, 5HT₅ antagonists or combinations thereof.

The assaying step a) and/or the measuring step b) of all the methods according to the present invention may further comprise:
i) contacting said sample with one or more binding agents to each of said biomarkers of the panel; or
ii) detecting in said sample autoantibodies specific to each of said biomarkers; or
iii) detecting in said sample by mass spectrometry each of said biomarkers of the panel, optionally by previously labelling said sample with one or more isobaric reactive mass labels; or
iv) detecting in said sample by 2D gel electrophoresis each of said biomarkers of the panel; or
iv) any combinations of i), ii), iii) or iv).

Preferably, the assaying in step a) and/or the measuring in step b) comprise:
i) detecting one or more fragments of said biomarkers in the panel and/or
ii) detecting one or more phosphorylated amino acids on tau comprising or having the amino acid sequence of SEQ ID NO: 29 or one or more fragments thereof; wherein when the phosphorylated amino acid on tau to be detected is T181, at least one more phosphorylated amino acid on tau or one or more fragments thereof is detected.

Optionally, the sample is immobilised on a solid support.

The sample to be assayed in the methods according to the present invention is selected from the group of cerebrospinal fluid (CSF), blood, plasma, serum, saliva, urine, tissue (e.g. brain tissue) or combinations thereof.

Preferably the sample is CSF or blood.

The subject to be diagnosed or assessed or treated may be an animal model (e.g. a rodent or a primate) of AD or of a tauopathy as described herein or a human subject. Preferably, the subject to be diagnosed, assessed or treated is a human subject.

### 2. Kits

The present invention also provides for kits comprising reagents for assaying and/or measuring in a sample the biomarkers of the panels according to the present invention.

Preferably, the kit allows the diagnosing, staging and assessment of response to a treatment for neurocognitive disorders, in particular Alzheimer's disease.

The reagents of the kits according to the invention may comprise one or more binding agents which specifically bind to the biomarkers of the panels described herein. Preferably, the one or more binding agents are primary antibodies, wherein each primary antibody specifically binds to:
i) a different protein of the panel and/or
ii) one or more phosphorylated amino acids of tau comprising or having amino acid sequence of SEQ ID NO: 29 or fragments thereof.

More preferably, the primary antibodies are one or more antibodies against protein phosphatase 1 regulatory subunit 14A and/or one or more antibodies against 2′,3′-cyclic-nucleotide 3′-phosphodiesterase. Other primary antibodies include antibodies against the other biomarkers of Groups A, B, C or D and of the proteins listed in Tables 5 to 13.

The primary antibodies may be immobilised on an assay plate, beads, microspheres or particles. Optionally, beads, microspheres or particles may be dyed, tagged or labelled. Optionally the assay plate is a planar array or microtitre multi-well plate.

When the kits comprise primary antibodies against the biomarkers of the panel, the kits may further comprise one or more secondary antibodies which specifically bind to said primary antibodies.

Optionally, the secondary antibodies may be labelled for example fluorescent labelled or tagged.

The kits according to the invention may further comprise one or more detection reagents for detecting the presence of the tagged secondary antibodies.

The sample is preferably selected from the group of cerebrospinal fluid (CSF), blood, plasma, serum, saliva, urine, tissue (e.g. brain tissue) or combinations thereof.

The kits of the invention allow to:
a) assay a sample obtained from a subject for biomarkers of a panel;
b) measure in said sample a concentration or an amount of each of the biomarkers of said panel;
c) determine whether that subject has a neurocognitive disorder, in particular Alzheimer's disease, by comparing said concentration or amount of each of the protein in said sample to reference concentrations or amounts of said proteins;
   wherein the panel of biomarkers is selected from a panel comprising:
   I) i) protein phosphatase 1 regulatory subunit 14A comprising or having the amino acid sequence of SEQ ID NO:1 or an isoform or a variant or a fragment thereof; and/or
ii) 2′,3′-cyclic-nucleotide 3′-phosphodiesterase comprising or having the amino acid sequence of SEQ ID NO:2 or an isoform a variant or a fragment thereof; or
   II) one or more biomarkers selected from Groups A, B, C or D; or
   III) tau or one or more fragments thereof, wherein tau:
i) comprises or has the amino acid sequence of SEQ ID NO:29 and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/5416 or S422; wherein when the phosphorylated amino acid on tau is T181, the panel comprises tau or one or more fragments thereof having at least one more phosphorylated amino acid; or
   IV) one or more, optionally two or more proteins selected from Tables 5, 6, 7, 8, 9, 10, 11, 12, 13 or combinations thereof; or
   V) combinations of I), II), III) and IV.

In particular, the kits according to the invention may instruct to assay (as in step a)) and/or to measure (as in step b)) the sample by:
i) contacting said sample with one or more binding agents to each of said biomarkers of the panel; or
ii) detecting in said sample autoantibodies specific to each of said biomarkers; or
iii) detecting in said sample by mass spectrometry each of said biomarkers of the panel, optionally by previously labelling said sample with one or more isobaric reactive mass labels; or
iv) detecting in said sample by 2D gel electrophoresis each of said proteins of the panel; or
iv) any combinations of i), ii), iii) or iv).

In yet another embodiment, the kits may comprise reagents suitable for preparing brain tissue, optionally for preparing formalin-fixed paraffin-embedded brain tissue sections.

The kit may additionally provide a reference which provides a quantitative measure by which determination of a concentration or amount of one or more biomarkers can be compared. The reference may indicate the amount or concentration of proteins which indicate the presence or staging or likelihood of developing a neurocognitive disorder such as a tauopathy in particular AD.

The kit may also comprise printed instructions for performing the methods according to the present invention.

In one embodiment, the kit may be for performance of a mass spectrometry assay and may comprise a set of reference peptides (e.g. SRM peptides) in an assay compatible format wherein each peptide in the set is uniquely representative of i) one or more of the biomarkers of Groups A, B, C or D; ii) phosphorylated tau comprising or having the amino acid sequence of SEQ ID NO: 29 or one or more fragments thereof; or iii) one or more of the proteins listed in Tables 5 to 13.

Preferably two or more of such unique peptides are used for each biomarker for which the kit is designed, and wherein each set of unique peptides are provided in known amounts which reflect the amount or concentration of such biomarker in a sample of a healthy subject.

Optionally, the kit may also provide protocols and reagents for the isolation and extraction of the biomarkers according to the invention from a sample, a purified preparation of a proteolytic enzyme such as trypsin and a detailed protocol of the method including details of the precursor mass and specific transitions to be monitored. The peptides may be synthetic peptides and may comprise one or more heavy isotopes of carbon, nitrogen, oxygen and/or hydrogen.

Optionally, the kits of the present invention may also comprise appropriate cells, vessels, growth media and buffers.

### 3. Detection and Measurement of Biomarkers

The panel of biomarkers described herein comprise both biomarkers where expression is modulated, i.e. quantitatively increased or decreased, and biomarkers which are exclusively present or absent, i.e. qualitatively expressed, in normal versus disease states. The degree to which expression differs in normal versus disease states need only be large enough to be visualised via standard characterisation techniques.

Methods for the detection and quantification of proteins are well known in the art and any suitable method may be employed.

In one embodiment, the biomarkers of the panel may be detected using a binding agent, such as an antibody, specific to that biomarker, for example in an ELISA assay or Western blotting.

Methods relating to the production of antibodies capable of specifically recognising one or more epitopes, including phosphorylated amino acids or amino acids carrying other post-translational modifications, of the individual biomarker in the panel described herein are known in the art. Such antibodies may include, but are not limited to, polyclonal antibodies, monoclonal antibodies (mAbs), humanised or chimeric antibodies, single chain antibodies, Fab fragments, F(ab′)2 fragments, fragments produced by a Fab expression library, anti-idiotypic (anti-Id) antibodies, and epitope-binding fragments of any of the above.

For the production of antibodies, various host animals may be immunised by injection with a protein, or a portion thereof. Such host animals may include, but are not limited to, rabbits, mice and rats. Various adjuvants may be used to increase the immunological response, depending on the host species, including active substances such as lysolecithin, Pluronic polyols, polyanions, peptides, oil emulsions, keyhole limpet hemocyamin, dinitrophenol, and potentially useful human adjuvant such as BCG bacille Calmette-Fuerin) and Corynebacterium parvum.

Polyclonal antibodies are heterogeneous populations of antibody molecules derived from the sera of animals immunised with an antigen, such as target proteins, or an antigenic functional derivative thereof. For the production of polyclonal antibodies, host animals such as those described above, may be immunised by injection with differentially expressed or pathway protein supplemented with adjuvants as also described above.

Monoclonal antibodies, which are homogeneous populations of antibodies to a particular antigen, may be obtained by any technique, which provides for the production of antibody molecules by continuous cell lines in culture. These include, but are not limited to, the hybridoma technique of Kohler and Milstein (1975, Nature 256; 495-497; and U.S. Pat. No. 4,376,110), the human β-cell hybridoma technique (Kosbor, et al., 1983, Immunology Today 4: 72; Cole, et al., 1983, Proc. Natl. Acad. Sci. USA 80; 2026-2030), and the EBV-hybridoma technique (Cole, et al., 1985, Monoclonal Antibodies and Cancer Therapy, Alan R. Liss Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of ‘chimeric antibodies’ (Morrison, et al., 1984, Proc. Natl. Acad. Sci. 81: 6851-6855; Neuberger, et al., 1984, Nature 312: 604-608; Takeda, et al., 1985, Nature 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242: 423-426; Huston, et al., 1988, Proc. Natl. Acad. Sci. USA 85: 5879-5883; and Ward, et al., 1989, Nature 334: 544-546) can be adapted to produce differentially expressed or pathway protein-single chain antibodies. Single chain antibodies are formed by linking the heavy and light chain fragments of the Fv region via an amino acid bridge, resulting in a single chain polypeptide.

Antibody fragments, which recognise specific epitopes, may be generated by known techniques. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternative, Fab expression libraries may be constructed (Huse, et al., 1989, Science 246: 1275-1281) to allow rapid and easy identification of monoclonal Fab fragments with the desired specificity.

In some embodiments of the methods described herein, the sample may be immobilised on a solid support for analysis. An antibody sandwich technique may be employed in which binding agents, such as antibodies, specific for the individual protein of the panel are immobilized on a solid support such as a planar surface or a microparticle bead and proteins of the panel are captured by the immobilised binding agents, such as immobilized antibodies. The captured proteins are then detected using a second binding agent, such as a secondary antibody, that may be directly labelled with a signal generating agent (enzyme, fluorescent tag, radiolabel etc.) or may be detected using further amplification (labelled secondary antibody, streptavidin/biotin systems with enzyme, fluorophore, radiolabel etc.). Other methods may include, but are not limited to, one-dimensional or two-dimensional (2D) gel electrophoresis of samples. Such methods are followed by transfer to a solid surface using techniques such as Western blotting and subsequent detection using antibodies specific for the proteins of the panel.

In other embodiments, autoantibodies to the biomarkers of the panel may be detected using the Western blotting approach described above using samples from a healthy subject, a patient or representative of AD, and then detecting the presence of auto-antibodies specific for the biomarkers that are present in the sample, but not in healthy subjects.

An example of a non-antibody binding agent is an aptamer. Examples of aptamers include nucleic acid aptamers and peptide aptamers.

Alternatively, the biomarkers of the panel may be detected by, amongst others, silver staining of 2D gel electrophoresis or mass spectrometry techniques including LS/MS/MS, MALDI-TOF, SELDI-TOF and TMT-SRM.

Other such standard characterisation techniques by which expression differences may be visualised are well known to those skilled in the art. These include successive chromatographic separations of fractions and comparisons of the peaks, capillary electrophoresis, separations using micro-channel networks, including on a micro-chip, SELDI analysis and qPST analysis.

Chromatographic separations can be carried out by high performance liquid chromatography as described in literature, the chromatogram being obtained in the form of a plot of absorbance of light at 280 nm against time of separation. The material giving incompletely resolved peaks is then re-chromatographed and so on.

Capillary electrophoresis may also be employed. The technique depends on applying an electric potential across the sample contained in a small capillary tube. The tube has a charged surface, such as negatively charged silicate glass. Oppositely charged ions (in this instance, positive ions) are attracted to the surface and then migrate to the appropriate electrode of the same polarity as the surface (in this instance, the cathode). In this electro-osmotic flow (EOF) of the sample, the positive ions move fastest, followed by uncharged material and negatively charged ions. Thus, proteins are separated essentially according to charge on them.

Micro-channel networks function similarly to capillaries and can be formed by photoablation of a polymeric material. In this technique, a UV laser is used to generate high energy light pulses that are fired in bursts onto polymers having suitable UV absorption characteristics, for example polyethylene terephthalate or polycarbonate. The incident photons break chemical bonds with a confined space, leading to a rise in internal pressure, mini-explosions and ejection of the ablated material, leaving behind voids which form micro-channels. The micro-channel material achieves a separation based on EOF, as for capillary electrophoresis. It is adaptable to micro-chip form, each chip having its own sample injector, separation column and electrochemical detector.

Surface enhanced laser desorption ionisation time of flight mass spectrometry (SELDI-TOF-MS) combined with ProteinChip technology can also provide a rapid and sensitive means of profiling biomarkers and is used as an alternative to 2D gel electrophoresis in a complementary fashion.

The ProteinChip system consists of aluminium chips to which protein samples can be selectively bound on the surface chemistry of the chip (eg. anionic, cationic, hydrophobic, hydrophilic etc). Bound biomarkers are then co-crystallised with a molar excess of small energy-absorbing molecules. The chip is then analysed by short intense pulses of N₂ 320 nm UV laser with protein separation and detection being by time of flight mass spectrometry. Spectral profiles of each group within an experiment are compared and any peaks of interest can be further analysed using techniques as described below to establish the identity of the protein of the panel.

Isotopic or isobaric Tandem Mass Tags® (TMT® Thermo Scientific, Rockford, USA) technology may also be used to detect proteins of the panel described herein. Briefly, the proteins in the samples for comparison are optionally digested, labelled with a stable isotope tag and quantified by mass spectrometry. In this way, expression of equivalent proteins in the different samples can be compared directly by comparing the intensities of their respective isotopic peaks or of reporter ions released from the TMT® reagents during fragmentation in a tandem mass spectrometry experiment.

Detection of the proteins of the panel described herein may be preceded by a depletion step to remove the most abundant proteins from the sample. The large majority of the protein composition of serum/plasma consists of just a few proteins. For example, albumin, which is present at a concentration of 35-50 mg/ml, represents approximately 54% of the total protein content with IgG adding other 16%. In contrast, proteins changing in response to disease, for example as a result of tissue leakage, may circulate at 10 ng/ml. This vast dynamic range of protein concentrations represents a major analytical challenge and to overcome the problem, a multiple affinity depletion column may be used to remove the most highly abundant proteins (e.g. the 2, 3, 4, 5, 6, 7, 8, 9 or 10 or more highly abundant proteins). This enables the detection of changes in lower abundance ranges because more starting material can be used and there is less interference from the highly abundant molecules. Such a depletion strategy can be applied before any detection method.

### 4. Examples

Certain aspects and embodiments of the invention will now be illustrated by way of example and with reference to the figures and tables described above. All documents mentioned in this specification are incorporated herein by reference in their entirety for all purposes.

All reagents for sample preparation were purchased from Sigma Aldrich® (Dorset, UK) unless stated. Tandem Mass Tags® (Thermo Scientific®); Acetonitrile (Fisher Scientific®, Loughborough, UK); Trypsin (Roche Diagnostics®, West Sussex, UK).

4.1 Proteins which Correlate with Neurofibrillary Tangle Pathology in the Brain (Braak Staging)

### Samples Preparation

Nine frozen tissue samples from inferior temporal cortex samples were dissected from larger frozen tissue sections. Samples were selected according to neurofibrillary tangle (NFT) pathology (=Braak staging) and thus represent all the phases of the AD-related NFT pathology.

The study group comprised one sample each for Braak stage 0, I and II, one sample for Braak stage III, two samples with Braak stage IV, one sample for Braak stage V and a further two samples with Braak stage 6.

The samples were processed and analysed according to the SysQuant® technology as described in details herein below, within one SysQuant® TMT 10plex experiment, which included all nine samples and one study reference.

Quantitative considerations have been performed following a statistical evaluation by a principal component analysis (PCA) of the data, which showed most significant differences in samples with higher Braak staging. Consequently to the PCA results, regulations have been calculated and statistically evaluated by comparing samples with Braak stages V/VI (severe group) to samples with Braak stages III/IV (moderate group).

The cortex samples in lysis buffer (8 M urea, 75 mM NaCl, 50 mM Tris, pH 8.2, protease and phosphatase inhibitors cocktail (Roche) were lysed by sonification (20% Amplitude for 20×1 second, pulsing on and off, on ice (4° C.)) and then centrifuged at 12,500 g for 10 min at 4° C. to eliminate tissue debris. Supernatant was transferred into new tubes and the protein concentration of the samples determined using the Bradford assay. Per sample, the same protein amount was used for all subsequent steps.

After lysis, a reference sample was generated by mixing of identical amounts of all nine individual samples. In particular, for each sample 2.5 mg of protein material were taken and adjusted to a concentration of 3 mg/ml. From each adjusted sample, 167 μL were taken and combined to generate a reference sample. The remaining 666 μL (=2 mg) were used for individual sample manipulation.

DTT was added to each sample (final concentration 5 mM) and incubate for 25 min at 56° C. under shaking to reduce disulfide bonds. Samples were then allowed to cool at room temperature before adding iodoacetamide (final concentration 14 mM) and incubated for 30 min at room temperature and in the dark to alkylate cysteine residues. Unreacted iodoacetamide was quenched by adding DTT to additional 5 mM and incubating 15 min at room temperature in the dark.

Samples were diluted with 25 mM Tris-HCl, pH 8.2, to reduce the concentration of urea to 1.6 M. Trypsin (Roche, UK) was added to a final minimum concentration of 5 ng/μL (trypsin to substrate ratio of at least 1:100) together with CaCl₂ (final concentration of 1 mM). Samples were incubated at 37° C. overnight with shaking (˜15-18 hours). Digested samples were allowed to cool to room temperature and digestion was stopped by acidification with TFA to 0.4% (vol/vol). Samples were centrifuged at 2,500 g for 10 min at room temperature and the pellet was discarded.

Samples were desalted using a 200 mg SepPak tC18 cartridges (Thermo Scientific UK) according to manufacturer's instructions and proteins were eluted with elution buffer (50% ACN, 50% H2O) and concentrated to dryness in Speedvac.

For TMT® labelling samples were re-solubilised into 567 μL of TEAB/ACN buffer for a final concentration of 2.0-2.5 mg total peptide amount per sample. TMT® labels from the TMT® 10plex regent set was added to each sample according to Table 14 below to give a final concentration of 15 mM TMT® in each sample.

TABLE 14

| Sample No. | Braak staging | TMT ® labelling |
|---|---|---|
| 1 | V | TMT⁶-126 |
| 2 | IV | TMT⁶-128 |
| 3 | VI | TMT⁶-129e |
| 4 | I | TMT⁶-127 |
| 5 | VI | TMT⁶-130 |
| 6 | 0 | TMT⁶-128e |
| 7 | II | TMT⁶-129 |
| 8 | IV | TMT⁶-127e |
| 9 | III | TMT⁶-130e |
| Ref. | N/A | TMT⁶-131 |

Ref. = Reference sample containing an aliquot of all the samples

Reaction were allowed to take place for 1 hr at room temperature. Hydroxylamine was added to each sample to a final concentration of 0.25% [w/v]hydroxylamine and incubated for 15 minutes. Samples were then diluted 1:3 with 2% TFA and then further diluted with water to reduce the concentration of ACN to below 5%. Samples were mixed in equal amounts to achieve one SysQuant10plex sample which was then split into two aliquots which were desalted (500 mg SepPak tC18 cartridges) and fractionated by SCX chromatography (3 mL/min as flow rate; Buffer A: Water+0.1% TFA; Buffer C: 7 mM KH2PO4, pH 2.65, 30% ACN (vol/vol); Buffer D: 7 mM KH2PO4, 350 mM KCl, pH 2.65, 30% ACN (vol/vol)) according to the protocol in Table 15.

TABLE 15

| Time | Buffer A | Buffer C | Buffer D |
|---|---|---|---|
| [min] | [%] | [%] | [%] |
| 0 | 0 | 100 | 0 |
| 2 | 0 | 100 | 0 |
| 35 | 0 | 75 | 25 |
| 36 | 0 | 0 | 100 |
| 46 | 0 | 0 | 100 |
| 47 | 100 | 0 | 0 |
| 57 | 100 | 0 | 0 |
| 58 | 0 | 100 | 0 |
| 67 | 0 | 100 | 0 |
| 68 | 0 | 100 | 0 |

Lyophilized peptides from desalting were re-suspended in 800 μL of buffer A, injected onto the HPLC system. Twelve fractions were collected and “smart pooled” by mixing fractions with low numbers of peptides with fractions containing large numbers of peptides to provide 6 final fractions with similar total peptide content and desalted.

Then, small portions were taken from each fraction to apply the subsequent analysis of non-enriched fractions. The remaining portion of the fractions were applied to enrichment for phosphopeptides by either the IMAC or the TiO₂ procedures, both well-known in the art. Dried fractions were transferred for LC-MS analysis

### LC-MS/MS Analysis

“Smart-pooled” fractions were analysed twice each by LC-MS/MS (double-shot workflow), with MS acquisition performed by Top Speed MS2 HCD method.

Peptides from non-enriched fractions were re-suspended in 100 μl 2% ACN/0.1% FA, then Sul per fraction was injected onto a 2 cm×75 um Acclaim PepMap100 pre-column, and separated using an EASY-Spray 50 cm×75 um ID, PepMap RSCL, C18, 2 um, on the EASY-nLC 1000 system (Thermo Fisher Scientific). Peptides were resolved using a 160 min separation gradient of 8 to 30% ACN/0.1% FA at 200 nL/min.

Peptides from all phospho-enriched fractions were re-suspended in 30 μl of 2% ACN/0.1% FA, then 5 μL per fraction injected and resolved using a 160 min separation gradient of 10 to 30% 0.1% FA in ACN at 200 nL/min.

Mass spectra were acquired on an Orbitrap Fusion™ Tribrid™ Mass Spectrometer (Thermo Fisher Scientific) for a total run time of 180 min using top speed higher collision induced dissociation (HCD) FTMS2 scans at 30,000 resolving power, following each FTMS scan (120,000 resolving power). HCD was carried out on the most intense ions from each FTMS scan, and then put on a dynamic exclusion list for 30 sec to avoid repeated sequencing of the same analyte. Each sample was analysed by two LC-MS/MS analytical repeats (double shot workflow).

### Computational MS

The acquired spectra were processed using Proteome Discoverer 1.4 (PD 1.4; Thermo Fisher Scientific) software using the human specific UniProtKB/Swiss-Prot database (88,647 sequence entries) downloaded on 22 Feb. 2014. The raw data was searched using the Sequest HT and Mascot (Matrix Mascot server 2.2.06) search algorithms within PD 1.4. MS raw data files that belonged to the 6 fractions of any enrichment arm of the SysQuant® workflow and those which belonged to the same analytical set of runs were submitted as an individual MudPit search each. Thus, in total four MudPit searches were performed. After filtering the search results at 1% false discovery rate (FDR) at the peptide level and at least one rank 1 peptide per protein, search results were exported to MS Excel files. This data was processed by defined scripts to list all identified peptides, and their protein origin.

### Data Analysis

All protein identification, peptide sequence, phosphorylation site information along with quantitative values and biological information relating to the peptide, protein and phosphorylation sites were assembled in a Microsoft Excel file (QuantSheet) to allow structured data analysis based on fold-change, significance, biological function and cellular localisation.

To evaluate the general data quality by bioinformatic means, i.e. to visualize the effect of Braak staging, and thus to guide subsequent quantitative computations, a principle component analysis (PCA) was carried out.

A thorough biological data interpretation was also performed and a targeted analysis of proteins of interest was applied, for example for tau protein and additional AD biomarkers Proteins implicated in neurodegeneration.

### 4.2 Quantification of Regulated Proteins in AD CSF Using TMTcalibrator™

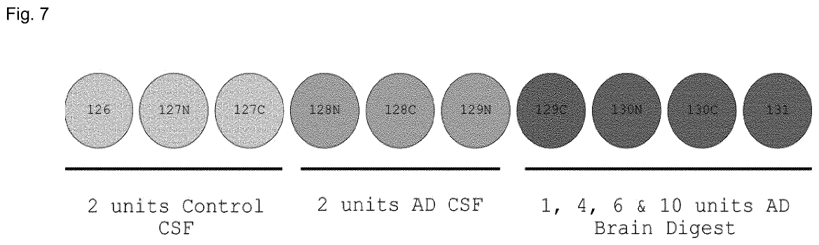

We also identified the presence and relative abundance of phosphorylated peptides of tau in human CSF drawn from controls that were biochemically negative for AD and those that were biochemically positive. Briefly, post-mortem collected pre-frontal cortex material from human subjects with moderate tau pathology (Braak stages III-IV; n=3) or severe tau pathology (Braak stages V-VI; n=3), performed in separate experiments, were pooled, digested with trypsin and labelled in four separate aliquots with TMT reagents TMT¹⁰-129C, 130N, 130C and 131. Each labelled aliquot from the same brain digest pool was mixed at a ratio of 0.3 mg:1.2 mg:1.8 mg:3.0 mg respectively to form a calibration standard. At the same time human cerebrospinal fluid samples (600 ug protein content (600 ul) per individual) from three non-cognitively impaired control individuals and three biochemically diagnosed cases of AD were digested with trypsin and labelled with TMT® reagents TMT¹⁰-126, 127N, 127C, 128N, 128C and 129N respectively and mixed to form the clinical test sample. Finally an equal volume of the calibration standard and clinical test sample were mixed to form the analytical sample according to FIG. 7.

Mass spectrometry and data analysis was carried out as described above in Section 4.1.

### 4.3 Effects of Tau Inhibition In Vivo

The inventors have also applied SysQuant to analyse protein and phosphorylation changes in the brains of TMHT (Thy-1 Mutated Human Tau) mice (developed by QPS® Austria, http://www.qps-austria.com) treated with small molecule inhibitors of the tau kinase Casein Kinase 1 delta (CK1d). Starting at 8.5 months (±2 weeks) of age, TMHT mice received CK1d inhibitors 2-Methyl-amino-3-[(4-fluorophenyl)carbonyl]indolizine-1-carboxamide (PS278-05), 5-(1,3-benzoxazol-2-yl)-4-(pyridine-4-yl)pyrimidin-2-amine (PS110), compound PF4800567 (3-[(3-Chlorophenoxy)methyl]-1-(tetrahydro-2H-pyran-4-yl)-1H-pyrazolo[3,4-d]pyrimidin-4-amine; Tocris®) or vehicle (0.5% w/v methylcellulose) for 8 weeks (54 applications) at a dosage of 30 mg/kg body weight, orally via gavage.

In total 48 animals were used and allocated to 4 treatment groups. Table 16 describes the animals, cohort and treatment group allocation, sex and age of the animals.

TABLE 16

| | | | | Start Age |
|---|---|---|---|---|
| Cohort | Group | Treatment | Sex | [months] |
| I | A | Vehicle | m | 8.75 |
| I | A | Vehicle | f | 8.75 |
| I | A | Vehicle | f | 8.75 |
| I | A | Vehicle | f | 8.75 |
| I | A | Vehicle | m | 8.42 |
| I | A | Vehicle | m | 8.42 |
| I | B | Cmp A | m | 8.75 |
| I | B | Cmp A | m | 8.75 |
| I | B | Cmp A | f | 8.45 |
| I | B | Cmp A | f | 8.45 |
| I | B | Cmp A | f | 8.45 |
| I | B | Cmp A | m | 8.32 |
| I | C | Cmp 324 | f | 8.75 |
| I | C | Cmp 324 | f | 8.75 |
| I | C | Cmp 324 | m | 8.75 |
| I | C | Cmp 324 | m | 8.75 |
| I | C | Cmp 324 | f | 8.75 |
| I | C | Cmp 324 | m | 8.45 |
| I | D | PF4800567 | m | 8.75 |
| I | D | PF4800567 | f | 8.75 |
| I | D | PF4800567 | f | 8.75 |
| I | D | PF4800567 | f | 8.45 |
| *I | D | PF4800567 | m | 8.42 |
| I | D | PF4800567 | m | 8.32 |
| II | A | Vehicle | f | 8.65 |
| II | A | Vehicle | f | 8.65 |
| II | A | Vehicle | m | 8.55 |
| II | A | Vehicle | f | 8.52 |
| II | A | Vehicle | f | 8.35 |
| II | A | Vehicle | m | 8.19 |
| II | B | Cmp A | f | 8.55 |
| II | B | Cmp A | f | 8.55 |
| II | B | Cmp A | f | 8.52 |
| II | B | Cmp A | m | 8.19 |
| II | B | Cmp A | m | 8.19 |
| II | B | Cmp A | f | 8.35 |
| II | C | Cmp 324 | m | 8.02 |
| II | C | Cmp 324 | m | 8.02 |
| II | C | Cmp 324 | f | 8.65 |
| II | C | Cmp 324 | f | 8.65 |
| II | C | Cmp 324 | f | 8.35 |
| II | C | Cmp 324 | f | 8.52 |
| II | D | PF4800567 | f | 8.35 |
| II | D | PF4800567 | f | 8.55 |
| II | D | PF4800567 | f | 8.55 |
| II | D | PF4800567 | m | 8.35 |
| II | D | PF4800567 | m | 8.35 |
| II | D | PF4800567 | f | 8.19 |

Cmp 324 = 5-(1,3-benzoxazol-2-yl)-4-(pyridine-4-yl)pyrimidin-2-amine;
Cmp A = 2-Methyl-amino-3-[(4-fluorophenyl)carbonyl]indolizine-1-carboxamide;
f = female;
m = male

Brain samples were collected within 10 minutes of euthanasia, washed in ice cold saline, transferred to a clean Eppendorf tube and immediately frozen in liquid nitrogen.

One hippocampus sample (left or right) from each of three animals in each of the vehicle, PS110 and PS278-05 groups were selected for analysis by SysQuant. A pool of all nine samples was also prepared as a reference channel. All TMT® labelling, mixing and mass spectrometry and bioinformatics studies were performed essentially as described in Section 4.1. For peptide and protein identification the UniProtKB mouse database was used, supplemented by inclusion of all human tau isoform entries.

All protein identification, peptide sequence, phosphorylation site information along with quantitative values and biological information relating to the peptide, protein and phosphorylation sites were assembled in a Microsoft Excel file (QuantSheet) to allow structured data analysis based on fold-change, significance, biological function and cellular localisation.

### 4.4 Detection of Phosphorylated Tau

The kinetic profile of tau phosphorylation in the various human and mouse samples studied in Sections 4.1, 4.2 and 4.3 was determined by a targeted data analysis of the respective QuantSheets. All peptides that matched the human MAPT (P10636; including P10636-8=SEQ ID NO:29) UniProt sequences were exported into a separate Microsoft Excel sheets.

Ion intensity values for each non-phosphorylated peptide were summed and the average calculated for each disease severity group (Braak Stage 0-II; Braak Stage III-IV; Braak Stage V-VI) to give a value for total tau expression. For each phosphorylated amino acid e.g. pT181, the ion intensity values of all peptides containing said phosphorylated amino acid were summed and the average calculated for each disease severity group (Braak Stage 0-II; Braak Stage III-IV; Braak Stage V-VI).

A total of 185 unique peptides from human tau were quantified in all nine brain samples with 35 high confidence (phosphoRS score>75%) phosphorylation sites. Of these, the levels of 74 peptides were significantly (p<0.05) regulated in the brains of patients with moderate (Braak stage III-IV) and severe (Braak stage V-VI) tau pathology Similarly, the expression levels of total tau and for each phosphorylated serine, threonine and tyrosine amino acid in mouse brains following treatment with vehicle control or the CK1d inhibitors PS278-05 and PS110 were calculated.

All peptides that matched the human MAPT (P10636; SEQ ID NO:29) UniProt sequence and/or the mouse MAPT (P10637) were exported into a separate Microsoft Excel sheet. Ion intensity values for each phosphopeptide were summed and the average calculated for each treatment group (Vehicle control, PS110, PS278-05). We quantified 124 unique peptides from human tau in all nine brain samples with 39 high confidence (phosphoRS score>75%) phosphorylation sites. Of these, the levels of 37 peptides were significantly (p<0.05) regulated in the hippocampi of mice treated with PS110 whilst 22 peptides were significantly (p<0.05) regulated in the hippocampi of mice treated with PS278-05.

Finally, the levels of total tau and for each phosphorylated serine, threonine and tyrosine amino acid in human CSF samples drawn from control and biochemically confirmed cases of AD were determined as above. All peptides that matched the human MAPT (P10636) UniProt sequence were exported into a separate Microsoft Excel sheet. Ion intensity values for each phosphopeptide were summed and the average calculated for each group (Control, AD). There were 65 quantified tau peptides in human CSF covering 27 high confidence phosphorylation sites.

In total, across the three studies we identified 44 unique phosphorylation sites on tau protein with 19 sites being quantified in all samples of human brain, mouse brain and human CSF. Table 4 collates all Tau phosphorylation sites identified by SysQuant® and TMTcalibrator™ in mouse and human brain tissue and human CSF. Amino acid numbering based on human 2N4R tau (Uniprot Accession Number P10636-8 with SEQ ID NO:29).

The method of measurement of these phosphorylated residues in tau protein are not intended to limit the invention. One or more of the sites may be measured using a binding agent such as an antibody or aptamer specific for the phosphorylated residue. However, for some sites the use of a binding agent will be less desirable due to the influence of adjacent phosphorylation on additional residues. One such case is the measurement of phosphorylated threonine 181 (2N4R tau numbering). Current diagnosis of AD is supported by the measurement of CSF levels of total tau and pT181 to yield a ratio. The larger the ratio the greater the probability that the patient has AD. In the present study we have identified that pT181 can be measured on two separate peptides in CSF. One is a singly phosphorylated peptide at threonine 181 whilst the other is triply phosphorylated at threonine 181, serine 184 and serine 185. It is probable that additional phosphorylation at S184 and S185 will interfere with the affinity of the binding agent reducing its ability to bind to the triply phosphorylated species and so under-representing the total amount of pT181 in CSF.

To overcome these limitations, phosphorylated tau is preferably measured using mass spectrometry. Any form of mass spectrometry capable of providing a relative or absolute quantification of each phosphorylation may be used. Such methods include but are not limited to data independent acquisition (DIA), data dependent acquisition (DDA), selected reaction monitoring (SRM), multiple reaction monitoring (MRM), or TMTcalibrator™. In each case a reference phosphopeptide that can be differentiated from the endogenous phosphotau peptide in the test sample using mass spectrometry is provided. The reference phosphopeptide may be provided from a biological sample and made distinct from endogenous phosphopeptides using an isotopic or isobaric mass tag. Alternatively the reference phosphopeptide may be generated from digestion of a recombinant tau protein manufactured using a synthetic lysine source that has several H², C¹³, N¹⁵, O¹⁸ atomic substitutions to ensure each tryptic peptide will have a mass greater than the naturally occurring equivalent phosphopeptide of at least 1 Dalton, preferably more than 2 Daltons and most preferably more than 5 Daltons. Fully synthetic peptides manufactured by sequential amino acid addition may be used wherein one or more amino acids within the peptide sequence contains atomic substitutions with H², C¹³, N¹⁵, O¹⁸ or other such appropriate stable heavy isotope.

Irrespective of the means of detection and quantification, assays intended for early diagnosis, prognosis of disease progression or monitoring of therapeutic effect may measure one, two or more different tau phosphopeptides.

### 4.5 Identification of Other Brain-Derived Proteins in Human CSF

In addition to identifying regulated peptides derived from tau protein, we also analysed the data from Example 4.2 to identify other disease-related proteins that are differentially expressed in human AD CSF. Briefly, the TMT® reporter ion intensities for all unmodified peptides matched to a unique UniProt accession number were summed and used to determine the relative expression of the relevant protein in human CSF. Based on the summed intensity values a log 2 ratio was calculated for each protein expression in control (n=3) and AD (n=3) groups. Statistical significance was calculated as a p-value based on the six independent protein quantification values using a two sample t-test. The resulting data matrix was exported to Microsoft Excel and filtered to select all proteins showing greater than 40% regulation (−0.5≤log 2≥0.5) and p≤0.05. This was performed for both TMTcalibrator™ experiments using brain calibrants with moderate and severe tau pathology. The list of regulated proteins including log 2 ratio and p-values is shown in Table 5.

Any of the proteins listed in Table 5 may be used as diagnostic and/or prognostic biomarkers of AD.

### 4.6 Results

The present inventors have surprisingly identified biomarkers which are highly regulated in the brain of patients with AD and/or which are present in the CSF and/or which are highly regulated in response to administration of casein kinase inhibitors. In addition, tau toxicity in the brain of the patients used in these experiments present elevated expression and hyperphosphorylation (tau toxicity).

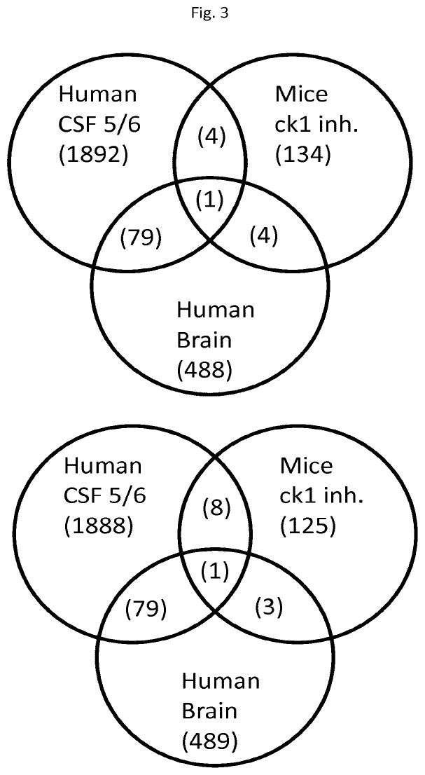

Surprisingly, when the data were analysed with respect to Braak stages V or VI, protein phosphatase 1 regulatory subunit 14A resulted to be the most upregulated protein (FIG. 3). Interestingly, levels of this protein first fell between mild (Braak stage 0-II) and moderate (Braak stage III/IV) before rising significantly in severe disease. Treatment with both casein kinase 1 delta inhibitors 5-(1,3-benzoxazol-2-yl)-4-(pyridine-4-yl)pyrimidin-2-amine or 2-Methyl-amino-3-[(4-fluorophenyl)carbonyl]indolizine-1-carboxamide resulted in an up-regulation of protein phosphatase 1 regulatory subunit 14A, apparently counteracting the drop in expression levels seen in moderate tau pathology in humans). FIG. 3 shows a Venn diagram for the analyses performed on the brains of patients with AD (label “Human Brain”), CSF of patients with AD at Braak stage V or VI (label “Human CSF 5/6) and on the brains of mice treated with 5-(1,3-benzoxazol-2-yl)-4-(pyridine-4-yl)pyrimidin-2-amine (upper diagram) or 2-Methyl-amino-3-[(4-fluorophenyl)carbonyl]indolizine-1-carboxamide (lower diagram) (label “Mice ck1 inh.). The numbers in the brackets below each of the labels indicate the number of proteins identified. As it can be seen in FIG. 3, there is only one protein which is upregulated in all the samples and this protein is protein phosphatase 1 regulatory subunit 14A.

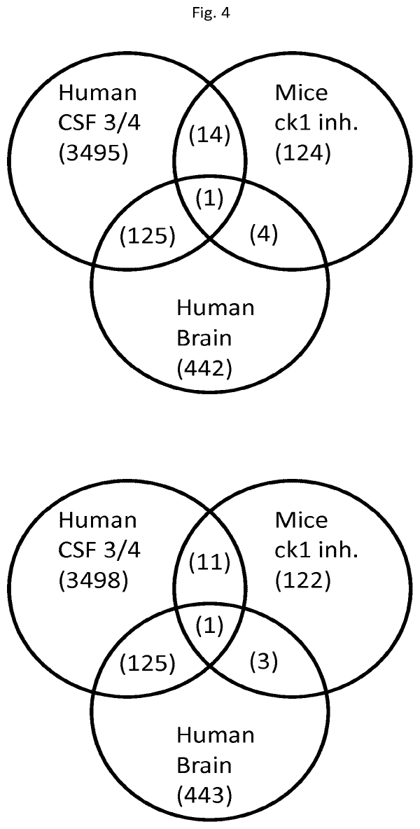

Even more surprisingly, when the data were analysed with respect to Braak stages V or VI, 2′,3′-cyclic-nucleotide 3′-phosphodiesterase resulted to be the most upregulated protein (FIG. 4). Interestingly, levels of this protein first fell between mild (Braak stage 0-II) and moderate (Braak stage III/IV) before rising significantly in severe disease. Again, in the parallel study in the TMHT model of tauopathy the type of treatment did not play a significant role with both compounds increasing the expression of the protein. FIG. 4 shows a Venn diagram for the analyses performed on the brains of patients with AD (label “Human Brain”), CSF of patients with AD at Braak stage 3 or 4 (label “Human CSF 3/4) and on the brains of mice treated with 5-(1,3-benzoxazol-2-yl)-4-(pyridine-4-yl)pyrimidin-2-amine (upper diagram) or 2-Methyl-amino-3-[(4-fluorophenyl)carbonyl]indolizine-1-carboxamide (lower diagram) (label “Mice ck1 inh.). As it can be seen in FIG. 4, there is only one protein which is upregulated in all the samples and this protein is 2′,3′-cyclic-nucleotide 3′-phosphodiesterase.

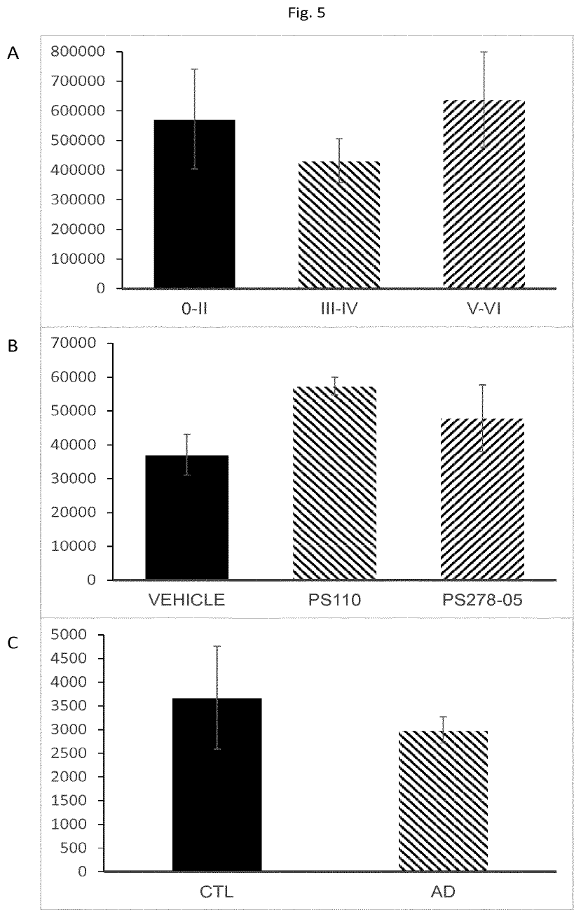

FIG. 5 shows a bar diagram (A) which depicts the levels of protein phosphatase 1 regulatory subunit 14A levels in human brain with mild (Braak 0-II) (n=3), moderate (Braak III/IV) or severe (Braak V/VI) tau pathology; the levels of protein phosphatase 1 regulatory subunit 14A are increased in the brain of human subjects with an advanced stage of the neurocognitive disorder (Braak V/VI). FIG. 5 diagram (B) shows that mouse brain from the TMHT model of human tauopathy treated orally with vehicle alone or vehicle including 30 mg/kg of casein kinase 1 delta inhibitors 5-(1,3-benzoxazol-2-yl)-4-(pyridin-4-yl)pyrimidin-2-amine and 2-Methyl-amino-3-[(4-fluorophenyl)carbonyl]indolizine-1-carboxamide responded to treatment altering the concentration or amount of protein phosphatase 1 regulatory subunit 14A. Finally, FIG. 5 diagram (C) shows that CSF from patients with biochemically diagnosed AD (n=3) have a decreased level of protein phosphatase 1 regulatory subunit 14A compared to cognitively affected non-AD controls (n=3).

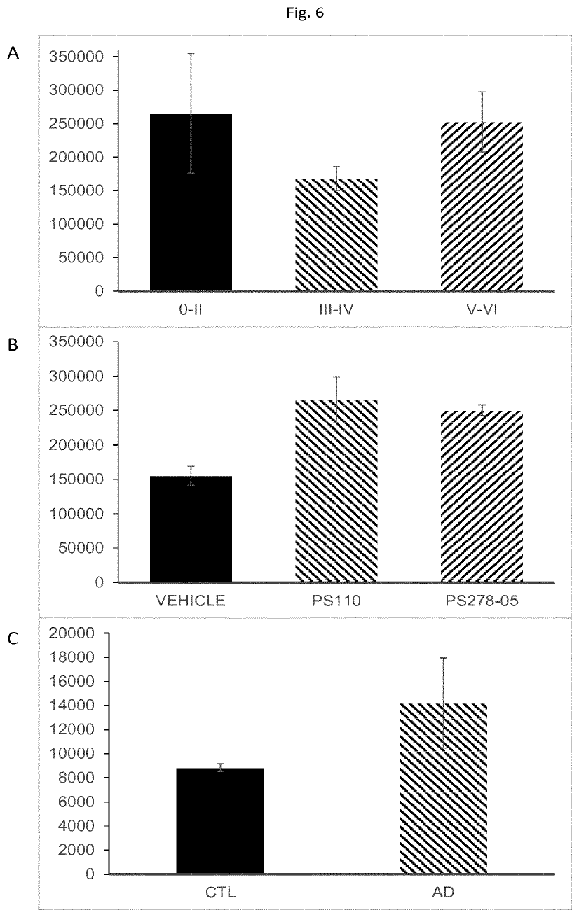

FIG. 6 shows a bar diagram (A) which depicts the levels of 2′,3′-cyclic-nucleotide 3′-phosphodiesterase levels in human brain with mild (Braak 0-II) (n=3), moderate (Braak III/IV) or severe (Braak V/VI) tau pathology; the levels of 2′,3′-cyclic-nucleotide 3′-phosphodiesterase are increased in the brain of human subjects with an advanced stage of the neurocognitive disorder (Braak V/VI). FIG. 6 diagram (B) shows that mouse brain from the TMHT model of human tauopathy treated orally with vehicle alone or vehicle including 30 mg/kg of casein kinase 1 delta inhibitors 5-(1,3-benzoxazol-2-yl)-4-(pyridin-4-yl)pyrimidin-2-amine and 2-Methyl-amino-3-[(4-fluorophenyl)carbonyl]indolizine-1-carboxamide responded to treatment altering the concentration or amount of protein 2′,3′-cyclic-nucleotide 3′-phosphodiesterase. Finally, FIG. 6 diagram (C) shows that CSF from patients with biochemically diagnosed AD (n=3) have a increased level of 2′,3′-cyclic-nucleotide 3′-phosphodiesterase compared to cognitively affected non-AD controls (n=3).

In summary, 1) both phosphatase 1 regulatory subunit 14A and 2′,3′-cyclic-nucleotide 3′-phosphodiesterase are elevated in the brain of subjects with an advanced stage of a neurocognitive disorder (Braak stage V or stage VI); 2) in the CSF of AD subjects protein phosphatase 1 regulatory subunit 14A is decreased whilst 2′,3′-cyclic-nucleotide 3′-phosphodiesterase is elevated in comparison to cognitively affected non-AD controls; and 3) both phosphatase 1 regulatory subunit 14A and 2′,3′-cyclic-nucleotide 3′-phosphodiesterase are upregulated in response to casein kinase 1 delta inhibitors 5-(1,3-benzoxazol-2-yl)-4-(pyridin-4-yl)pyrimidin-2-amine and 2-Methyl-amino-3-[(4-fluorophenyl)carbonyl]indolizine-1-carboxamide.

Finally, additional proteins from Groups A, B, C, D and tables 5 to 13 can also be selected together with phosphatase 1 regulatory subunit 14A and 2′,3′-cyclic-nucleotide 3′-phosphodiesterase to form enlarged biomarker panels for the diagnoses, staging and assessment of treatment of a neurocognitive disorder, in particular of AD.

1-36. (canceled)

37. A panel of biomarkers comprising tau or one or more fragments thereof, wherein tau:
i) comprises or has the amino acid sequence of SEQ ID NO:29, and
ii) comprises one or more, optionally two or more, phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422;
wherein when the phosphorylated amino acid on tau is T181, the panel comprises tau or one or more fragments thereof having at least one more phosphorylated amino acid.

38. The panel of claim 37, wherein tau or one or more fragments thereof comprises:
phosphorylated amino acids S61, S64, T181, S184, S202, S205, T231 and/or S235;
phosphorylated amino acids selected from S61, S64, S199, S205, and S396;
phosphorylated amino acid S61;
phosphorylated amino acid S64;
phosphorylated amino acid S199;
phosphorylated amino acid S205;
phosphorylated amino acid S396;
phosphorylated amino acids S61 and S64;
phosphorylated amino acids S61 and S199;
phosphorylated amino acids S61 and S205;
phosphorylated amino acids S61 and S396;
phosphorylated amino acids S64 and S199;
phosphorylated amino acids S64 and S205;
phosphorylated amino acids S64 and S396;
phosphorylated amino acids S199 and S205;
phosphorylated amino acids S199 and S396;
phosphorylated amino acids S205 and S396;
phosphorylated amino acids S61, S64 and S199;
phosphorylated amino acids S61, S64 and S205;
phosphorylated amino acids S61, S64 and S396;
phosphorylated amino acids S61, S199 and S205;
phosphorylated amino acids S61, S199 and S396;
phosphorylated amino acids S61, S205 and S396;
phosphorylated amino acids S64, S199 and S205;
phosphorylated amino acids S64, S199 and S396;
phosphorylated amino acids S64, S205 and S396;
phosphorylated amino acids S199, S205 and S396;
phosphorylated amino acids S61, S64, S199 and S205;
phosphorylated amino acids S61, S64, S199 and S396;
phosphorylated amino acids S61, S199, S205 and S396; or
phosphorylated amino acids S61, S64, S199, S205, and S396.

39. A panel of biomarkers comprising a) at least one biomarker selected from Table 5, Table 6, Table 7, Table 8, or Table 9; and b) at least one biomarker selected from tau or one or more fragments thereof, wherein tau:
i) comprises or has the amino acid sequence of SEQ ID NO:29, and
ii) comprises one or more, optionally two or more phosphorylated amino acids selected from T39, S46, T50, T52, T56, S61, T63, S64, S68, T69, S113, T181, S184, S185, S191, S195, S198, S199, S202, S205, S208, S210, T212, S214, T217, T231, S235, S237, S238, S258, S262, S285, S289, S356, Y394, S396, S400, T403, S404, S409, S412, S413, T414/S416 or S422;
wherein when the phosphorylated amino acid on tau is T181, the panel comprises tau or one or more fragments thereof having at least one more phosphorylated amino acid.

40. A method for diagnosing a neurocognitive disorder in a subject, the method comprising:
a) assaying a sample obtained from said subject for the biomarkers of the panel of claim 39;
b) measuring in said sample a concentration or an amount of each of the biomarkers of said panel; and
c) determining whether that subject has a neurocognitive disorder by comparing said concentration or amount of each of the biomarkers of the panel in said sample with reference concentrations or amounts of said biomarkers.

41. A method for staging a neurocognitive disorder in a subject, the method comprising:
a) assaying a sample obtained from said subject for the biomarkers of the panel of claim 39;
b) measuring in said sample a concentration or an amount of each of the biomarkers of said panel; and
c) determining the stage of the neurocognitive disorder in said subject by comparing said concentration or amount of each of the biomarkers of the panel in said sample with reference concentrations or amounts of said biomarkers.

42. A method for assessing in a subject the likelihood of developing a neurocognitive disorder, the method comprising:
a) assaying a sample obtained from said subject for the biomarkers of the panel of claim 39;
b) measuring in said sample a concentration or an amount of each of the biomarkers of said panel; and
c) determining whether that subject is likely to develop a neurocognitive disorder by comparing said concentration or amount of each of the biomarkers of the biomarker panel in said sample with reference concentrations or amounts of said biomarkers.

43. A method for treating a neurocognitive disorder in a subject, the method comprising:
a) assaying a sample obtained from said subject for the biomarkers of the panel of claim 39;
b) measuring in said sample a concentration or an amount of each of the biomarkers of said panel;
c) determining whether that subject has a neurocognitive disorder by comparing said concentration or amount of each of the biomarkers in said sample with reference concentrations or amounts of said biomarkers; and
d) administering to said subject a drug for treating the neurocognitive disorder.

44. A method for assessing the response to a drug for treating a neurocognitive disorder in a subject, wherein the subject has been treated or is being treated with said drug, the method comprising:
a) assaying a sample obtained from a subject for the biomarkers of the panel of claim 39;
b) measuring in said sample a concentration or an amount of each of the biomarkers of said panel; and
c) determining whether that subject has responded or is responding to said drug by comparing said concentration or amount of each of the biomarkers in said sample with reference concentrations or amounts of said biomarkers.

45. The method according to claim 40, wherein the sample comprises cerebrospinal fluid (CSF), blood, plasma, serum, saliva, urine, tissue, or a combination thereof.

46. The method according to claim 40, wherein the sample is CSF or blood.

47. The method according to claim 40, wherein the subject is a human subject.

48. A kit comprising reagents for assaying and/or measuring in a sample the biomarkers of the panel of claim 37.

49. The kit according to claim 48, wherein the reagents comprise one or more binding agents which specifically bind to the biomarkers of the panels.

50. The kit according to claim 49, wherein the one or more binding agents are primary antibodies, wherein each primary antibody specifically binds to:
i) a different biomarker of the panel, and/or
ii) one or more phosphorylated amino acids of tau comprising or having the amino acid sequence of SEQ ID NO: 29, or fragments thereof.

51. The kit according to claim 48, wherein said sample is selected from the group of cerebrospinal fluid (CSF), blood, plasma, serum, saliva, urine, tissue, or a combination thereof.


SEQUENCE LISTING (31 sequences)

SEQ ID NO: 1 - protein, 147 aa, Homo sapiens
MAAQRLGKRVLSKLQSPSRARGPGGSPGGLQKRHARVTVKYDRRELQRRLDVEKWIDGRLEELYRGMEADMPDEINIDELLELESEEERSRKIQGLLKSCGKPVEDFIQELLAKLQGLHRQPGLRQPSPSHDGSLSPLQDRARTAHP

SEQ ID NO: 2 - protein, 421 aa, Homo sapiens
MNRGFSRKSHTFLPKIFFRKMSSSGAKDKPELQFPFLQDEDTVATLLECKTLFILRGLPGSGKSTLARVIVDKYRDGTKMVSADAYKITPGARGAFSEEYKRLDEDLAAYCRRRDIRILVLDDTNHERERLEQLFEMADQYQYQVVLVEPKTAWRLDCAQLKEKNQWQLSADDLKKLKPGLEKDFLPLYFGWFLTKKSSETLRKAGQVFLEELGNHKAFKKELRQFVPGDEPREKMDLVTYFGKRPPGVLHCTTKFCDYGKAPGAEEYAQQDVLKKSYSKAFTLTISALFVTPKTTGARVELSEQQLQLWPSDVDKLSPTDNLPRGSRAHITLGCAADVEAVQTGLDLLEILRQEKGGSRGEEVGELSRGKLYSLGNGRWMLTLAKNMEVRAIFTGYYGKGKPVPTQGSRKGGALQSCTII

SEQ ID NO: 3 - protein, 195 aa, Homo sapiens
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 4 - protein, 968 aa, Homo sapiens
MDDSEVESTASILASVKEQEAQFEKLTRALEEERRHVSAQLERVRVSPQDANPLMANGTLTRRHQNGRFVGDADLERQKFSDLKLNGPQDHSHLLYSTIPRMQEPGQIVETYTEEDPEGAMSVVSVETSDDGTTRRTETTVKKVVKTVTTRTVQPVAMGPDGLPVDASSVSNNYIQTLGRDFRKNGNGGPGPYVGQAGTATLPRNFHYPPDGYSRHYEDGYPGGSDNYGSLSRVTRIEERYRPSMEGYRAPSRQDVYGPQPQVRVGGSSVDLHRFHPEPYGLEDDQRSMGYDDLDYGMMSDYGTARRTGTPSDPRRRLRSYEDMIGEEVPSDQYYWAPLAQHERGSLASLDSLRKGGPPPPNWRQPELPEVIAMLGFRLDAVKSNAAAYLQHLCYRNDKVKTDVRKLKGIPVLVGLLDHPKKEVHLGACGALKNISFGRDQDNKIAIKNCDGVPALVRLLRKARDMDLTEVITGTLWNLSSHDSIKMEIVDHALHALTDEVIIPHSGWEREPNEDCKPRHIEWESVLTNTAGCLRNVSSERSEARRKLRECDGLVDALIFIVQAEIGQKDSDSKLVENCVCLLRNLSYQVHREIPQAERYQEAAPNVANNTGPHAASCFGAKKGKDEWFSRGKKPIEDPANDTVDFPKRTSPARGYELLFQPEVVRIYISLLKESKTPAILEASAGAIQNLCAGRWTYGRYIRSALRQEKALSAIADLLTNEHERVVKAASGALRNLAVDARNKELIGKHAIPNLVKNLPGGQQNSSWNFSEDTVISILNTINEVIAENLEAAKKLRETQGIEKLVLINKSGNRSEKEVRAAALVLQTIWGYKELRKPLEKEGWKKSDFQVNLNNASRSQSSHSYDDSTLPLIDRNQKSDKKPDREEIQMSNMGSNTKSLDNNYSTPNERGDHNRTLDRSGDLGDMEPLKGTTPLMQDEGQESLEEELDVLVLDDEGGQVSYPSMQKI

SEQ ID NO: 5 - protein, 229 aa, Homo sapiens
MADDFGFFSSSESGAPEAAEEDPAAAFLAQQESEIAGIENDEGFGAPAGSHAAPAQPGPTSGAGSEDMGTTVNGDVFQEANGPADGYAAIAQADRLTQEPESIRKWREEQRKRLQELDAASKVTEQEWREKAKKDLEEWNQRQSEQVEKNKINNRIADKAFYQQPDADIIGYVASEEAFVKESKEETPGTEWEKVAQLCDFNPKSSKQCKDVSRLRSVLMSLKQTPLSR

SEQ ID NO: 6 - protein, 359 aa, Homo sapiens
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 7 - protein, 944 aa, Homo sapiens
MAAVAAVAARRRRSWASLVLAFLGVCLGITLAVDRSNFKTCEESSFCKRQRSIRPGLSPYRALLDSLQLGPDSLTVHLIHEVTKVLLVLELQGLQKNMTRFRIDELEPRRPRYRVPDVLVADPPIARLSVSGRDENSVELTMAEGPYKIILTARPFRLDLLEDRSLLLSVNARGLLEFEHQRAPRVSQGSKDPAEGDGAQPEETPRDGDKPEETQGKAEKDEPGAWEETFKTHSDSKPYGPMSVGLDFSLPGMEHVYGIPEHADNLRLKVTEGGEPYRLYNLDVFQYELYNPMALYGSVPVLLAHNPHRDLGIFWLNAAETWVDISSNTAGKTLFGKMMDYLQGSGETPQTDVRWMSETGIIDVFLLLGPSISDVFRQYASLTGTQALPPLFSLGYHQSRWNYRDEADVLEVDQGFDDHNLPCDVIWLDIEHADGKRYFTWDPSRFPQPRTMLERLASKRRKLVAIVDPHIKVDSGYRVHEELRNLGLYVKTRDGSDYEGWCWPGSAGYPDFTNPTMRAWWANMFSYDNYEGSAPNLFVWNDMNEPSVFNGPEVTMLKDAQHYGGWEHRDVHNIYGLYVHMATADGLRQRSGGMERPFVLARAFFAGSQRFGAVWTGDNTAEWDHLKISIPMCLSLGLVGLSFCGADVGGFFKNPEPELLVRWYQMGAYQPFFRAHAHLDTGRREPWLLPSQHNDIIRDALGQRYSLLPFWYTLLYQAHREGIPVMRPLWVQYPQDVTTFNIDDQYLLGDALLVHPVSDSGAHGVQVYLPGQGEVWYDIQSYQKHHGPQTLYLPVTLSSIPVFQRGGTIVPRWMRVRRSSECMKDDPITLFVALSPQGTAQGELFLDDGHTFNYQTRQEFLLRRFSFSGNTLVSSSADPEGHFETPIWIERVVIIGAGKPAAVVLQTKGSPESRLSFQHDPETSVLVLRKPGINVASDWSIHLR

SEQ ID NO: 8 - protein, 425 aa, Homo sapiens
MASSSPDSPCSCDCFVSVPPASAIPAVIFAKNSDRPRDEVQEVVFVPAGTHTPGSRLQCTYIEVEQVSKTHAVILSRPSWLWGAEMGANEHGVCIGNEAVWTKEPVGEGEALLGMDLLRLALERSSSAQEALHVITGLLEHYGQGGNCLEDAAPFSYHSTFLLADRTEAWVLETAGRLWAAQRIQEGARNISNQLSIGTDISAQHPELRTHAQAKGWWDGQGAFDFAQIFSLTQQPVRMEAAKARFQAGRELLRQRQGGITAEVMMGILRDKESGICMDSGGFRTTASMVSVLPQDPTQPCVHFLTATPDPSRSVFKPFIFGMGVAQAPQVLSPTFGAQDPVRTLPRFQTQVDRRHTLYRGHQAALGLMERDQDRGQQLQQKQQDLEQEGLEATQGLLAGEWAPPLWELGSLFQAFVKRESQAYA

SEQ ID NO: 9 - protein, 412 aa, Homo sapiens
MAAALLARASGPARRALCPRAWRQLHTIYQSVELPETHQMLLQTCRDFAEKELFPIAAQVDKEHLFPAAQVKKMGGLGLLAMDVPEELGGAGLDYLAYAIAMEEISRGCASTGVIMSVNNSLYLGPILKFGSKEQKQAWVTPFTSGDKIGCFALSEPGNGSDAGAASTTARAEGDSWVLNGTKAWITNAWEASAAVVFASTDRALQNKGISAFLVPMPTPGLTLGKKEDKLGIRGSSTANLIFEDCRIPKDSILGEPGMGFKIAMQTLDMGRIGIASQALGIAQTALDCAVNYAENRMAFGAPLTKLQVIQFKLADMALALESARLLTWRAAMLKDNKKPFIKEAAMAKLAASEAATAISHQAIQILGGMGYVTEMPAERHYRDARITEIYEGTSEIQRLVIAGHLLRSYRS

SEQ ID NO: 10 - protein, 494 aa, Homo sapiens
MAMSLPGSRRTSAGSRRRTSPPVSVRDAYGTSSLSSSSNSGSYKGSDSSPTPRRSMKYTLCSDNHGIKPPTPEQYLTPLQQKEVCIRHLKARLKDTQDRLQDRDTEIDDLKTQLSRMQEDWIEEECHRVEAQLALKEARKEIKQLKQVIDTVKNNLIDKDKGLQKYFVDINIQNKKLETLLHSMEVAQNGMAKEDGTGESAGGSPARSLTRSSTYTKLSDPAVCGDRQPGDPSSGSAEDGADSGFAAADDTLSRTDALEASSLLSSGVDCGTEETSLHSSFGLGPRFPASNTYEKLLCGMEAGVQASCMQERAIQTDFVQYQPDLDTILEKVTQAQVCGTDPESGDRCPELDAHPSGPRDPNSAVVVTVGDELEAPEPITRGPTPQRPGANPNPGQSVSVVCPMEEEEEAAVAEKEPKSYWSRHYIVDLLAVVVPAVPTVAWLCRSQRRQGQPIYNISSLLRGCCTVALHSIRRISCRSLSQPSPSPAGGGSQL

SEQ ID NO: 11 - protein, 377 aa, Homo sapiens
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 12 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 13 - protein, 426 aa, Homo sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 14 - protein, 453 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQVRPEHPAETEYDSLYPEDDL

SEQ ID NO: 15 - protein, 240 aa, Homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 16 - protein, 245 aa, Homo sapiens
MNGRADFREPNAEVPRPIPHIGPDYIPTEEERRVFAECNDESFWFRSVPLAATSMLITQGLISKGILSSHPKYGSIPKLILACIMGYFAGKLSYVKTCQEKFKKLENSPLGEALRSGQARRSSPPGHYYQKSKYDSSVSGQSSFVTSPAADNIEMLPHYEPIPFSSSMNESAPTGITDHIVQGPDPNLEESPKRKNITYEELRNKNRESYEVSLTQKTDPSVRPMHERVPKKEVKVNKYGDTWDE

SEQ ID NO: 17 - protein, 609 aa, Homo sapiens [sequence omitted]

SEQ ID NO: 18 - protein, 319 aa, Homo sapiens
MASSAAGCVVIVGSGVIGRSWAMLFASGGFQVKLYDIEQQQIRNALENIRKEMKLLEQAGSLKGSLSVEEQLSLISGCPNIQEAVEGAMHIQECVPEDLELKKKIFAQLDSIIDDRVILSSSTSCLMPSKLFAGLVHVKQCIVAHPVNPPYYIPLVELVPHPETAPTTVDRTHALMKKIGQCPMRVQKEVAGFVLNRLQYAIISEAWRLVEEGIVSPSDLDLVMSEGLGMRYAFIGPLETMHLNAEGMLSYCDRYSEGIKHVLQTFGPIPEFSRATAEKVNQDMCMKVPDDPEHLAARRQWRDECLMRLAKLKSQVQPQ

SEQ ID NO: 19 - protein, 1391 aa, Homo sapiens
MPSSLLGAAMPASTSAAALQEALENAGRLIDRQLQEDRMYPDLSELLMVSAPNNPTVSGMSDMDYPLQGPGLLSVPNLPEISSIRRVPLPPELVEQFGHMQCNCMMGVFPPISRAWLTIDSDIFMWNYEDGGDLAYFDGLSETILAVGLVKPKAGIFQPHVRHLLVLATPVDIVILGLSYANLQTGSGVLNDSLSGGMQLLPDPLYSLPTDNTYLLTITSTDNGRIFLAGKDGCLYEVAYQAEAGWFSQRCRKINHSKSSLSFLVPSLLQFTFSEDDPILQIAIDNSRNILYTRSEKGVIQVYDLGQDGQGMSRVASVSQNAIVSAAGNIARTIDRSVFKPIVQIAVIENSESLDCQLLAVTHAGVRLYFSTCPFRQPLARPNTLTLVHVRLPPGFSASSTVEKPSKVHRALYSKGILLMAASENEDNDILWCVNHDTFPFQKPMMETQMTAGVDGHSWALSAIDELKVDKIITPLNKDHIPITDSPVVVQQHMLPPKKFVLLSAQGSLMFHKLRPVDQLRHLLVSNVGGDGEEIERFFKLHQEDQACATCLILACSTAACDREVSAWATRAFFRYGGEAQMRFPTTLPPPSNVGPILGSPVYSSSPVPSGSPYPNPSFLGTPSHGIQPPAMSTPVCALGNPATQATNMSCVTGPEIVYSGKHNGICIYFSRIMGNIWDASLVVERIFKSGNREITAIESSVPCQLLESVLQELKGLQEFLDRNSQFAGGPLGNPNTTAKVQQRLIGFMRPENGNPQQMQQELQRKFHEAQLSEKISLQAIQQLVRKSYQALALWKLLCEHQFTIIVAELQKELQEQLKITTFKDLVIRDKELTGALIASLINCYIRDNAAVDGISLHLQDICPLLYSTDDAICSKANELLQRSRQVQNKTEKERMLRESLKEYQKISNQVDLSNVCAQYRQVRFYEGVVELSLTAAEKKDPQGLGLHFYKHGEPEEDIVGLQAFQERLNSYKCITDTLQELVNQSKAAPQSPSVPKKPGPPVLSSDPNMLSNEEAGHHFEQMLKLSQRSKDELFSIALYNWLIQVDLADKLLQVASPFLEPHLVRMAKVDQNRVRYMDLLWRYYEKNRSFSNAARVLSRLADMHSTEISLQQRLEYIARAILSAKSSTAISSIAADGEFLHELEEKMEVARIQLQIQETLQRQYSHHSSVQDAVSQLDSELMDITKLYGEFADPFKLAECKLAIIHCAGYSDPILVQTLWQDIIEKELSDSVTLSSSDRMHALSLKIVLLGKIYAGTPRFFPLDFIVQFLEQQVCTLNWDVGFVIQTMNEIGVPLPRLLEVYDQLFKSRDPFWNRMKKPLHLLDCIHVLLIRYVENPSQVLNCERRRFTNLCLDAVCGYLVELQSMSSSVAVQAITGNFKSLQAKLERLH

SEQ ID NO: 20 - protein, 183 aa, Homo sapiens
MSQKPAKEGPRLSKNQKYSEHFSIHCCPPFTFLNSKKEIVDRKYSICKSGCFYQKKEEDWICCACQKTRTSRRAKSPQRPKQQPAAPPAVVRAPAKPRSPPRSERQPRSPPRSERQPRSPPRSERQPRSPPRSERQPRPRPEVRPPPAKQRPPQKSKQQPRSSPLRGPGASRGGSPVKASRFW

SEQ ID NO: 21 - protein, 283 aa, Homo sapiens
MVSAAAPSLLILLLLLLGSVPATDARSVPLKATFLEDVAGSGEAEGSSASSPSLPPPWTPALSPTSMGPQPITLGGPSPPTNFLDGIVDFFRQYVMLIAVVGSLAFLLMFIVCAAVITRQKQKASAYYPSSFPKKKYVDQSDRAGGPRAFSEVPDRAPDSRPEEALDSSRQLQADILAATQNLKSPTRAALGGGDGARMVEGRGAEEEEKGSQEGDQEVQGHGVPVETPEAQEEPCSGVLEGAVVAGEGQGELEGSLLLAQEAQGPVGPPESPCACSSVHPSV

SEQ ID NO: 22 - protein, 265 aa, Homo sapiens
MCMVIFAPLFAIFAFATCGGYSGGLRLSVDCVNKTESNLSIDIAFAYPFRLHQVTFEVPTCEGKERQKLALIGDSSSSAEFFVTVAVFAFLYSLAATVVYIFFQNKYRENNRGPLIDFIVTVVFSFLWLVGSSAWAKGLSDVKVATDPKEVLLLMSACKQPSNKCMAIHSPVMSSLNTSVVFGFLNFILWAGNIWFVFKETGWHSSGQRYLSDPMEKHSSSYNQGGYNQDSYGSSSGYSQQASLGPTSDEFGQQPTGPTSFTNQI

SEQ ID NO: 23 - protein, 1584 aa, Homo sapiens
MSLTSWFLVSSGGTRHRLPREMIFVGRDDCELMLQSRSVDKQHAVINYDASTDEHLVKDLGSLNGTFVNDVRIPEQTYITLKLEDKLRFGYDTNLFTVVQGEMRVPEEALKHEKFTIQLQLSQKSSESELSKSASAKSIDSKVADAATEVQHKTTEALKSEEKAMDISAMPRGTPLYGQPSWWGDDEVDEKRAFKTNGKPEEKNHEAGTSGCGIDAKQVEEQSAAANEEVLFPFCREPSYFEIPTKEFQQPSQITESTIHEIPTKDTPSSHITGAGHASFTIEFDDSTPGKVTIRDHVTKFTSDQRHKSKKSSPGTQDLLGIQTGMMAPENKVADWLAQNNPPQMLWERTEEDSKSIKSDVPVYLKRLKGNKHDDGTQSDSENAGAHRRCSKRATLEEHLRRHHSEHKKLQKVQATEKHQDQAVTSSAHHRGGHGVPHGKLLKQKSEEPSVSIPFLQTALLRSSGSLGHRPSQEMDKMLKNQATSATSEKDNDDDQSDKGTYTIELENPNSEEVEARKMIDKVFGVDDNQDYNRPVINEKHKDLIKDWALSSAAAVMEERKPLTTSGFHHSEEGTSSSGSKRWVSQWASLAANHTRHDQEERIMEFSAPLPLENETEISESGMTVRSTGSATSLASQGERRRRTLPQLPNEEKSLESHRAKVVTQRSEIGEKQDTELQEKETPTQVYQKDKQDADRPLSKMNRAVNGETLKTGGDNKTLLHLGSSAPGKEKSETDKETSLVKQTLAKLQQQEQREEAQWTPTKLSSKNVSGQTDKCREETFKQESQPPEKNSGHSTSKGDRVAQSESKRRKAEEILKSQTPKGGDKKESSKSLVRQGSFTIEKPSPNIPIELIPHINKQTSSTPSSLALTSASRIRERSESLDPDSSMDTTLILKDTEAVMAFLEAKLREDNKTDEGPDTPSYNRDNSISPESDVDTASTISLVTGETERKSTQKRKSFTSLYKDRCSTGSPSKDVTKSSSSGAREKMEKKTKSRSTDVGSRADGRKFVQSSGRIRQPSVDLTDDDQTSSVPHSAISDIMSSDQETYSCKPHGRTPLTSADEHVHSKLEGSKVTKSKTSPVVSGSSSKSTTLPRPRPTRTSLLRRARLGEASDSELADADKASVASEVSTTSSTSKPPTGRRNISRIDLLAQPRRTRLGSLSARSDSEATISRSSASSRTAEAIIRSGARLVPSDKFSPRIRANSISRLSDSKVKSMTSAHGSASVNSRWRRFPTDYASTSEDEFGSNRNSPKHTRLRTSPALKTTRLQSAGSAMPTSSSFKHRIKEQEDYIRDWTAHREEIARISQDLALIAREINDVAGEIDSVTSSGTAPSTTVSTAATTPGSAIDTREELVDRVFDESLNFRKIPPLVHSKTPEGNNGRSGDPRPQAAEPPDHLTITRRRTWSRDEVMGDNLLLSSVFQFSKKIRQSIDKTAGKIRILFKDKDRNWDDIESKLRAESEVPIVKTSSMEISSILQELKRVEKQLQAINAMIDPDGTLEALNNMGFPSAMLPSPPKQKSSPVNNHHSPGQTPTLGQPEARALHPAAVSAAAEFENAESEADFSIHFNRFNPDGEEEDVTVQE

SEQ ID NO: 24 - protein, 381 aa, Homo sapiens
MASPPACPSEEDESLKGCELYVQLHGIQQVLKDCIVHLCISKPERPMKFLREHFEKLEKEENRQILARQKSNSQSDSHDEEVSPTPPNPVVKARRRRGGVSAEVYTEEDAVSYVRKVIPKDYKTMTALAKAISKNVLFAHLDDNERSDIFDAMFPVTHIAGETVIQQGNEGDNFYVVDQGEVDVYVNGEWVTNISEGGSFGELALIYGTPRAATVKAKTDLKLWGIDRDSYRRILMGSTLRKRKMYEEFLSKVSILESLEKWERLTVADALEPVQFEDGEKIVVQGEPGDDFYIITEGTASVLQRRSPNEEYVEVGRLGPSDYFGEIALLLNRPRAATVVARGPLKCVKLDRPRFERVLGPCSEILKRNIQRYNSFISLTV

SEQ ID NO: 25 - protein, 400 aa, Homo sapiens
MGVLMSKRQTVEQVQKVSLAVSAFKDGLRDRPSIRRTGELPGSRRGTVEGSVQEVQEEKEAEAGTSVVQEESSAGRAAWERLRDGRGVEPEEFDRTSRFTPPAFIRPTRKLDDDKPPEICLEPREPVVNDEMCDVCEVWTAESLFPCRVCTRVFHDGCLRRMGYIQGDSAAEVTEMAHTETGWSCHYCDNINLLLTEEEMYSLTETFQRCKVIPDCSLTLEDFLRYRHQAAKRGDRDRALSEEQEEQAARQFAALDPEHRGHIEWPDFLSHESLLLLQQLRPQNSLLRLLTVKERERARAAFLARGSGSTVSEAECRRAQHSWFCKRFPEAPSCSVSISHVGPIADSSPASSSSKSQDKTLLPTEQESRFVDWPTFLQENVLYILAARPNSAAIHLKPPG

SEQ ID NO: 26 - protein, 446 aa, Homo sapiens
MRECLSIHIGQAGIQIGDACWELYCLEHGIQPNGVVLDTQQDQLENAKMEHTNASFDTFFCETRAGKHVPRALFVDLEPTVIDGIRTGQHRSLFHPEQLLSGKEDAANNYARGRYSVGSEVIDLVLERTRKLAEQCGGLQGFLIFRSFGGGTGSGFTSLLMERLTGEYSRKTKLEFSVYPAPRISTAVVEPYNSVLTTHSTTEHTDCTFMVDNEAVYDICHRKLGVECPSHASINRLVVQVVSSITASLRFEGPLNVDLIEFQTNLVPYPRIHFPMTAFAPIVSADKAYHEQFSVSDITTACFESSNQLVKCDPRLGKYMACCLLYRGDVVPKEVNAAIAATKSRHSVQFVDWCPTGFKVGINNRPPTVMPGGDLAKVHRSICMLSNTTAIVEAWARLDHKFDLMYAKRAFLHWYLREGMEEAEFLEAREDLAALERDYEEVAQSF

SEQ ID NO: 27 - protein, 345 aa, Homo sapiens
MPRIDADLKLDFKDVLLRPKRSSLKSRAEVDLERTFTFRNSKQTYSGIPIIVANMDTVGTFEMAAVMSQHSMFTAIHKHYSLDDWKLFATNHPECLQNVAVSSGSGQNDLEKMTSILEAVPQVKFICLDVANGYSEHFVEFVKLVRAKFPEHTIMAGNVVTGEMVEELILSGADIIKVGVGPGSVCTTRTKTGVGYPQLSAVIECADSAHGLKGHIISDGGCTCPGDVAKAFGAGADFVMLGGMFSGHTECAGEVFERNGRKLKLFYGMSSDTAMNKHAGGVAEYRASEGKTVEVPYKGDVENTILDILGGLRSTCTYVGAAKLKELSRRATFIRVTQQHNTVFS

SEQ ID NO: 28 - protein, 176 aa, Homo sapiens
MDLPDSASRVFCGRILSMVNTDDVNAIILAQKNMLDRFEKTNEMLLNFNNLSSARLQQMSERFLHHTRTLVEMKRDLDSIFRRIRTLKGKLARQHPEAFSHIPEASFLEEEDEDPIPPSTTTTIATSEQSTGSCDTSPDTVSPSLSPGFEDLSHVQPGSPAINGRSQTDDEEMTGE

SEQ ID NO: 29 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 30 - protein, 161 aa, Homo sapiens [sequence omitted]

SEQ ID NO: 31 - protein, 79 aa, Homo sapiens
MYYKFSGFTQKLAGAWASEAYSPQGLKPVVSTEAPPIIFATPTKLTSDSTVYDYAGKNKVPELQKFFQVREDTLQQKSL